(19) United States
(12) Patent Application Publication
(10) Pub. No.: US 2006/0135403 A1
(43) Pub. Date: Jun. 22, 2006

(54) Therapeutic formulations for the treatment of beta-amyloid related diseases

(76) Inventors: Francine Gervais, IIe Bizard (CA); Francesco Bellini, Town of Mount Royal (CA)

Correspondence Address: CLARK & ELBING LLP, 101 FEDERAL STREET, BOSTON, MA, 02110, US

(21) Appl. No.: 10/540,763

(22) PCT Filed: Dec. 24, 2003
(86) PCT No.: PCT/CA03/02021

§ 371 Date: Nov. 18, 2005

Related U.S. Application Data
(60) Provisional application No. 60/436,379, filed on Dec. 24, 2002.
(60) Provisional application No. 60/482,214, filed on Jun. 23, 2003.

(51) Int. Cl. A61K 38/54

(52) U.S. Cl. 514/2

(57) ABSTRACT

The method is used for preventing or treating an amyloid-β related disease in a subject. The method comprises administering to a subject in need thereof an effective amount of a first agent that prevents or treats amyloid-β related disease, and a second agent that is (i) a peptide or peptidomimetic that modulates amyloid-β fibril formulation or induces a prophylactic or therapeutic immune response against amyloid-β fibril formulation, or (ii) an immune system modulator that pi-events or inhibits amyloid-β fibril formulation.

### BACKGROUND OF THE INVENTION

Alzheimer's disease is a devastating disease of the brain that results in progressive memory loss leading to dementia, physical disability, and death over a relatively long period of time. With the aging populations in developed countries, the number of Alzheimer's patients is reaching epidemic proportions.

People suffering from Alzheimer's disease develop a progressive dementia in adulthood, accompanied by three main structural changes in the brain: diffuse loss of neurons in multiple parts of the brain; accumulation of intracellular protein deposits termed neurofibrillary tangles; and accumulation of extracellular protein deposits termed amyloid or senile plaques, surrounded by misshapen nerve terminals (dystrophic neurites). A main constituent of these amyloid plaques is the amyloid-β peptide (Aβ), a 39-43 amino acid protein that is produced through cleavage of the β-amyloid precursor protein (APP). Extensive research has been conducted on the relevance of Aβ deposits in Alzheimer's disease (see, e.g., Selkoe, Trends in Cell Biology 8, 447-453 (1998)). Aβ naturally arises from the metabolic processing of APP in the endoplasmic reticulum (ER), the Golgi apparatus, or the endosomal-lysosomal pathway, and most is normally secreted as a 40 (Aβ1-40) or 42 (Aβ1-42) amino acid peptide (Selkoe, Annu. Rev. Cell Biol. 10, 373-403 (1994)). A role for Aβ as a primary cause for Alzheimer's disease is supported by the presence of extracellular Aβ deposits in senile plaques of Alzheimer's disease, the increased production of Aβ in cells harboring mutant Alzheimer's disease associated genes (e.g., amyloid precursor protein, presenilin I, and presenilin II genes), and the toxicity of extracellular soluble (oligomeric) or fibrillar Aβ to cells in culture (see, e.g., Gervais, Eur. Biopharm. Review, 40-42 (2001); and May, DDT 6, 459-462 (2001)). Although symptomatic treatments exist for Alzheimer's disease, this disease cannot be prevented or cured at this time.

Alzheimer's disease is characterized by diffuse and neuritic plaques, cerebral angiopathy, and neurofibrillary tangles. Plaque and blood vessel amyloid is believed to be formed by the deposition of insoluble Aβ amyloid protein, which may be described as diffuse or fibrillary. Both soluble oligomeric Aβ and fibrillar Aβ are also believed to be neurotoxic and inflammatory. Amyloid fibrils, once deposited, can become toxic to the surrounding cells. For example, Aβ fibrils organized as senile plaques have been shown to be associated with dead neuronal cells and microgliosis in patients with Alzheimer's disease. When tested in vitro, Aβ peptide was shown to be capable of triggering an activation process of microglia (brain macrophages), which would explain the presence of microgliosis and brain inflammation found in the brains of patients with Alzheimer's disease. Once these amyloids have formed, there is no known, widely accepted therapy or treatment that significantly dissolves the amyloid deposits in situ.

### SUMMARY OF THE INVENTION

The invention provides methods of preventing or treating amyloid-β related diseases in subjects (e.g., human subjects), which involve administering to subjects in need thereof an effective amount of a first agent that prevents or treats amyloid-β related disease (e.g., by preventing or inhibiting amyloid-β fibril formation, neurodegeneration, or cellular toxicity), and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation. The amyloid-β can be an amyloidogenic peptide produced from β-amyloid precursor protein having, e.g., 39-43 amino acids.

Diseases that can be prevented or treated according to the invention include, for example, Alzheimer's disease (e.g., sporadic (non-hereditary) or familial (hereditary) Alzheimer's disease), mild cognitive impairment, mild-to-moderate cognitive impairment, vascular dementia, cerebral amyloid angiopathy, hereditary cerebral hemorrhage, senile dementia, Down's syndrome, inclusion body myositis, age-related macular degeneration, or conditions associated with Alzheimer's disease, such as, for example, hypothyroidism, cerebrovascular disease, cardiovascular disease, memory loss, anxiety, behavioral dysfunctions, neurological conditions, and psychological conditions.

Examples of behavioral dysfunctions that can be treated or prevented include apathy, aggression, and incontinence; examples of neurological conditions that can be treated or prevented include Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, and posthypoxic damage; and examples of psychological conditions that can be treated or prevented include depression, delusions, illusions, hallucinations, sexual disorders, weight loss, psychosis, sleep disturbances, insomnia, behavioral disinhibition, poor insight, suicidal ideation, depressed mood, irritability, anhedonia, social withdrawal, and excessive guilt.

Subjects treated according to the methods of the invention can have, for example, a genomic mutation in an amyloid precursor protein gene, an ApoE gene, or a presenilin gene, and/or amyloid-β deposits.

The first agent noted above can function by any of a number of possible mechanisms. In specific examples, the agent prevents or inhibits β-amyloid fibril formation; prevents β-amyloid peptide, in-its soluble, oligomeric form, or in its fibrillar form, from binding or adhering to a cell surface and causing cell damage or toxicity, blocks amyloid-induced cellular toxicity or microglial activation; blocks amyloid-induced neurotoxicity; reduces the rate or amount of β-amyloid aggregation, fibril formation, or deposition; slows the rate of amyloid-β fibril formation or deposition; lessens the degree of amyloid-β deposition; inhibits, reduces, or prevents amyloid-β fibril formation; inhibits amyloid-β induced inflammation; enhances the clearance of amyloid-β from the brain; alters the equilibrium of amyloid-β between the cerebrospinal fluid or brain and the plasma and decreases the amount of amyloid-β in the brain versus the equilibrium distribution in an untreated subject; reverses or favors deposition of amyloid in a subject having amyloid deposits; favors plaque clearance or slows deposition in a subject having amyloid deposits; decreases the amyloid-β concentration in the brain of a subject versus an untreated subject; penetrates into the brain; maintains soluble amyloid in a non-fibrillar form; increases the rate of clearance of soluble amyloid from the brain of a subject versus an untreated subject; or inhibits or reduces an interation between amyloid-β and a cell surface constituent.

The first agent can be, for example, a substituted or unsubstituted alkylsulfonic acid, a substituted or unsubstituted alkylsulfuric acid, a substituted or unsubstituted alkylthiosulfonic acid, a substituted or unsubstituted alkylthiosulfuric acid, a substituted or unsubstituted lower alkylsulfonic acid, a (substituted- or unsubstituted-amino)-substituted alkylsulfonic acid, a (substituted- or unsubstituted-amino)-substituted lower alkylsulfonic acid, a substituted or unsubstituted straight-chain alkylsulfonic acid, a substituted or unsubstituted cycloalkylsulfonic acid, a substituted or unsubstituted branched-chain alkylsulfonic acid, or an ester or amide thereof, including pharmaceutically acceptable salts thereof.

In one example of such a first agent, the amino substituent has the formula —NR^{a}R^{b}, where R^{a} and R^{b} are each independently hydrogen, an alkyl group, an aryl group, or a heterocyclyl group, or R^{a} and R^{b}, taken together with the nitrogen atom to which they are attached, form a heterocyclic moiety having from 3 to 8 atoms in the ring. As an example, the heterocyclic moiety can include a piperidinyl or pyrrolidinyl group. In addition, the amino substituent noted above can include an alkylamino or dialkylamino group. Further, the alkylsulfonic acid can include an alkyl group substituted with at least a group of the formula —SO₃H or —SO₃⁻X⁺, where X⁺ is a cationic group (e.g., a hydrogen atom, a sodium atom, or an amino group) at physiologic pH. In yet further examples, the alkylsulfonic acid is substituted with a straight or branched alkyl or cycloalkyl group, or one of the following —NH₂, —SO₃H, —OSO₃H, —CN, —NO₂, —F, —Cl, —Br, —I, —CH₂OCH₃, —OCH₃, —SH, —SCH₃, —OH, or —CO₂H, or the alkylsulfonic acid is substituted with a substituent selected from the group consisting of halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl groups.

As an example, the first agent can be a compound or mixture of compounds of the following formula: 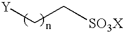
where Y is —NR^{a}R^{b} or —SO₃⁻X⁺, n is an integer from I to 5, and X⁺is hydrogen or a cationic group.

More specific examples include compounds having the following structures: 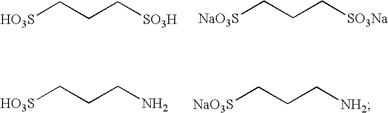
and pharmaceutically acceptable salts thereof, as well as 3-amino-1-propanesulfonic acid and pharmaceutically acceptable salts thereof.

In the methods of the invention, the first and second agents can be administered to a subject together, optionally, in a single pharmaceutical composition, or they can be administered sequentially. Further, at least one of the first and the second agents can be orally administered to a subject.

The second agent used in the methods of the invention can be a peptide or peptidomimetic compound that reduces or inhibits amyloid-β fibril formation. For example, the peptide can include hydrophobic amino acids and bind to the hydrophobic region of an amyloid-β peptide, thus blocking β-amyloid fibril formation. Optionally, the peptide can include one or more modifying groups that enhance the ability of the peptide to block amyloid fibril formation. Further, the peptide can be, e.g., an all D peptide as defined herein.

The second agent can also be administered to induce a prophylactic or therapeutic immune response against amyloid-β fibril formation. Optionally, such a method can further include administration of an adjuvant. Further, the second agent can be an immune system modulator selected from, for example, the group consisting of antibodies, antibody fragments, T-cells, B-cells, NK cells, NKT cells, dendritic cells, macrophages, basophils, monocytes, and components of the complement pathway.

The methods of the invention can also include administration of a third or further agent. For example, agents such as adrenergic, anti-adrenergic, anti-androgen, anti-anginal, anti-anxiety, anticonvulsant, antidepressant, anti-epileptic, antihyperlipidemic, antihyperlipoproteinemic, antihypertensive, anti-inflammatory, antiobessional, antiparkinsonian, antipsychotic, adrenocortical steroid; adrenocortical suppressant; aldosterone antagonist; amino acid; anabolic steroid; analeptic; androgen; blood glucose regulator; cardioprotectant; cardiovascular; cholinergic agonist or antagonist; cholinesterase deactivator or inhibitor; cognition adjuvant or enhancer; dopaminergic; enzyme inhibitor, estrogen, free oxygen radical scavenger; GABA agonist; glutamate antagonist; hormone; hypocholesterolemic; hypolipidemic; hypotensive; immunizing; immunostimulant; monoamine oxidase inhibitor, neuroprotective; NMDA antagonist; AMPA antagonist, competitive or non-competitive NMDA antagonist; opioid antagonist; potassium channel opener; non-hormonal sterol derivative; post-stroke and post-head trauma treatment; prostaglandin; psychotropic; relaxant; sedative; sedative-hypnotic; selective adenosine antagonist; serotonin antagonist; serotonin inhibitor; selective serotonin uptake inhibitor; serotonin receptor antagonist; sodium and calcium channel blocker; steroid; stimulant; and thyroid hormone or inhibitor agents can be used.

According to the methods of the present invention, the concentration of amyloid-β or tau in the cerebrospinal fluid of a treated subject can change as compared to the concentration in the cerebrospinal fluid of an untreated subject or the treated subject prior to treatment; the level of amyloid-β peptides in the plasma of a treated subject can be modulated as compared to the level in the plasma of an untreated subject or the treated subject prior to treatment; or the level of amyloid-β peptides in the cerebrospinal fluid of a treated subject can be lowered as compared to the level in an untreated subject or the treated subject prior to treatment.

In a more specific embodiment, the invention provides methods of preventing or treating an amyloid-β related disease in a subject, involving administration to a subject in need thereof an effective amount of 3-amino-1-propanesulfonic acid, and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation.

The invention also includes pharmaceutical compositions for treating subjects as described herein (e.g., human subjects) that include a first agent that prevents or treats amyloid-β related disease (e.g., by preventing or inhibiting amyloid-β fibril formation, neurodegeneration, or cellular toxicity), and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation. The amyloid-β can be an amyloidogenic peptide produced from β-amyloid precursor protein having, e.g., 39-43 amino acids.

Such pharmaceutical compositions can include the first agent and the second agent packaged in separate containers for sale or delivery to consumers. Alternatively, the first agent and the second agent can be dissolved in a liquid pharmaceutically acceptable carrier or can be present as a homogenous mixture in a capsule or pill. In addition, a compound that increases the cerebral bioavailability of either the first agent or the second agent can be included.

The pharmaceutical compositions of the invention can be used in the prevention and treatment of amyloid-β related diseases such as, for example, Alzheimer's disease (e.g., sporadic (non-hereditary) or familial (hereditary) Alzheimer's disease), mild cognitive impairment, mild-to-moderate cognitive impairment, vascular dementia, cerebral amyloid angiopathy, hereditary cerebral hemorrhage, senile dementia, Down's syndrome, inclusion body myositis, age-related macular degeneration, and conditions associated with Alzheimer's disease including, for example, hypothyroidism, cerebrovascular disease, cardiovascular disease, memory loss, anxiety, behavioral dysfunctions, neurological conditions, and psychological conditions.

As is noted above, examples of behavioral dysfunctions that can be treated or prevented include apathy, aggression, and incontinence; examples of neurological conditions that can be treated or prevented include Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, and posthypoxic damage; and examples of psychological conditions that can be treated or prevented include depression, delusions, illusions, hallucinations, sexual disorders, weight loss, psychosis, sleep disturbances, insomnia, behavioral disinhibition, poor insight, suicidal ideation, depressed mood, irritability, anhedonia, social withdrawal, and excessive guilt.

The pharmaceutical compositions can be used, for example, to treat subjects having a genomic mutation in an amyloid precursor protein gene, an ApoE gene, or a presenilin gene, and/or amyloid-β deposits.

The first agent of the compositions of the invention can function by any of a number of possible mechanisms. In specific examples, the agent prevents or inhibits. β-amyloid fibril formation; prevents β-amyloid peptide, in its soluble, oligomeric form, or in its fibrillar form, from binding or adhering to a cell surface and causing cell damage or toxicity; blocks amyloid-induced cellular toxicity or microglial activation; blocks amyloid-induced neurotoxicity; reduces the rate or amount of β-amyloid aggregation, fibril formation, or deposition; slows the rate of amyloid-β fibril formation or deposition; lessens the degree of amyloid-β deposition; inhibits, reduces, or prevents amyloid-β fibril formation; inhibits amyloid-β induced inflammation; enhances the clearance of amyloid-β from the brain; alters the equilibrium of amyloid-β between the cerebrospinal fluid or brain and the plasma and decreases the amount of amyloid-β in the brain versus the equilibrium distribution in an untreated subject; reverses or favors deposition of amyloid in a subject having amyloid deposits; favors plaque clearance or slows deposition in a subject having amyloid deposits; decreases the amyloid-β concentration in the brain of a subject versus an untreated subject; penetrates into the brain; maintains soluble amyloid in a non-fibrillar form; increases the rate of clearance of soluble amyloid from the brain of a subject versus an untreated subject; or inhibits or reduces an interation between amyloid-β and a cell surface constituent.

The first agent of the pharmaceutical compositions of the invention can be a substituted or unsubstituted alkylsulfonic acid, a substituted or unsubstituted alkylsulfuric acid, a substituted or unsubstituted alkylthiosulfonic acid, a substituted or unsubstituted alkylthiosulfuric acid, a substituted or unsubstituted lower alkylsulfonic acid, a (substituted- or unsubstituted-amino)-substituted alkylsulfonic acid, a (substituted- or unsubstituted-amino)-substituted lower alkylsulfonic acid, a substituted or unsubstituted straight-chain alkylsulfonic acid, a substituted or unsubstituted cycloalkylsulfonic acid, a substituted or unsubstituted branched-chain alkylsulfonic acid, or an ester or amide thereof, including pharmaceutically acceptable salts thereof.

In one example of such a first agent, the amino substituent has the formula —NR^{a}R^{b}, where R^{a} and R^{b} are each independently hydrogen, an alkyl group, an aryl group, or a heterocyclyl group, or R^{a} and R^{b}, taken together with the nitrogen atom to which they are attached, form a heterocyclic moiety having from 3 to 8 atoms in the ring. As an example, the heterocyclic moiety includes a piperidinyl or pyrrolidinyl group. In addition, the amino substituent noted above can include an alkylamino or dialkylamino group. Further, the alkylsulfonic acid can include an alkyl group substituted with at least a group of the formula —SO₃H or —SO₃⁻X⁺, where X⁺ is a cationic group (e.g., hydrogen atom, a sodium atom, or an amino group) at physiologic pH. In yet further examples, the alkylsulfonic acid is substituted with a straight or branched alkyl or cycloalkyl group, or —NH₂, —SO₃H, —OSO₃H, —CN, —NO₂, —F, —Cl, —Br, —I, —CH₂OCH₃, —OCH₃, —SH, —SCH₃, —OH, or —CO₂H, or the alkylsulfonic acid is substituted with a substituent selected from the group consisting of halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl groups.

As an example, the first agent can be a compound or mixture of compounds within the following formula: 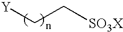
where Y is —NR^{a}R^{b} or —SO₃⁻X⁺, n is an integer from 1 to 5, and X+is hydrogen or a cationic group.

More specific examples include compounds having the following structures: 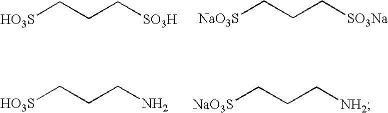
and pharmaceutically acceptable salts thereof, as well as 3-amino-1-propanesulfonic acid and pharmaceutically acceptable salts thereof.

The second agent of the pharmaceutical compositions of the invention can be a peptide or peptidomimetic compound that reduces or inhibits amyloid-β fibril formation. For example, the peptide can include hydrophobic amino acids and bind to the hydrophobic region of an amyloid-β peptide, thus blocking β-amyloid fibril formation. Such a peptide can, optionally, include one or more modifying groups that enhance the ability of the peptide to block amyloid fibril formation. Further, the peptide can be an all D peptide, as defined herein.

The second agent peptide or peptidomimetic of the pharmaceutical compositions of the invention can be administered to induce a prophylactic or therapeutic immune response against amyloid-β fibril formation. Optionally, pharmaceutical compositions for this purpose can also include an adjuvant. The second agent can also be an immune system modulator selected from, for example, the group consisting of antibodies, antibody fragments, T-cells, B-cells, NK cells, NKT cells, dendritic cells, macrophages, basophils, monocytes, and components of the complement pathway.

The pharmaceutical compositions of the invention can also include, optionally, a third or further agent. For example, the pharmaceutical compositions can include one or more of the following: adrenergic, anti-adrenergic, anti-androgen, anti-anginal, anti-anxiety, anticonvulsant, antidepressant, anti-epileptic, antihyperlipidemic, antihyperlipoproteinemic, antihypertensive, anti-inflammatory, antiobessional, antiparkinsonian, antipsychotic, adrenocortical steroid; adrenocortical suppressant; aldosterone antagonist; amino acid; anabolic steroid; analeptic; androgen; blood glucose regulator; cardioprotectant; cardiovascular; cholinergic agonist or antagonist; cholinesterase deactivator or inhibitor, cognition adjuvant or enhancer; dopaminergic; enzyme inhibitor, estrogen, free oxygen radical scavenger; GABA agonist; glutamate antagonist; hormone; hypocholesterolemic; hypolipidemic; hypotensive; immunizing; immunostimulant; monoamine oxidase inhibitor, neuroprotective; NMDA antagonist; AMPA antagonist, competitive or-non-competitive NMDA antagonist; opioid antagonist; potassium channel opener; non-hormonal sterol derivative; post-stroke and post-head trauma treatment; prostaglandin; psychotropic; relaxant; sedative; sedative-hypnotic; selective adenosine antagonist; serotonin antagonist; serotonin inhibitor; selective serotonin uptake inhibitor; serotonin receptor antagonist; sodium and calcium channel blocker; steroid; stimulant; and thyroid hormone and inhibitor agents.

In a specific embodiment, the invention provides a pharmaceutical composition including an effective amount of 3-amino-1-propanesulfonic acid, and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation.

Also included in the invention are kits that include a first agent that prevents or treats amyloid-β related disease, and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloidβ fibril formation. Specific examples of each of components of such a kit are provided throughout this application.

The invention also includes the use of a first agent and a second agent in the preparation of a pharmaceutical composition for the treatment or prevention of an amyloidβ disease, in which the first agent prevents or treats amyloid-β related disease, and the second agent is (i) a peptide or peptidomimetic that modulates amyloidβ fibril formation or induces a prophylactic or therapeutic immune response against amyloid-β fibril formation, or (ii) an immune system modulator that prevents or inhibits amyloid-β fibril formation.

Other features of the invention will be apparent from the following Detailed Description, the Drawings, and the Claims.

### BRIEF DESCRIPTION OF THE DRAWINGS

FIG. 1 is a graph showing the change in the levels of Aβ as determined by ELISA in cerebrospinal fluid samples from patients who underwent a 3-month treatment (time 0 vs. time 3 months) with a test sulfonic acid drug at daily doses of 100 mg, 200 mg, and 300 mg.

FIG. 2 is a graph showing the amount of a test sulfonic acid drug in cerebrospinal fluid samples from patients treated with three different dosages of a test sulfonic acid drug.

### DETAILED DESCRIPTION OF THE INVENTION

The invention pertains to pharmaceutical compositions, kits, and methods of use thereof for the prevention and treatment of amyloid-β diseases. The pharmaceutical compositions and kits include a first agent that prevents or treats amyloid-β diseases by, e.g., preventing or inhibiting amyloid-β fibril formation, neurodegeneration, or cellular toxicity, and a second agent that is also an active pharmaceutical ingredient; that is, the second agent is therapeutic and its function is beyond that of an inactive ingredient, such as a pharmaceutical carrier, preservative, diluent, or buffer. The pharmaceutical compositions and kits of the invention also can include one or more additional therapeutic agents that can be useful in treating or preventing an amyloid-β related or other neurological diseases. These additional agents can exert their biological effects by mechanisms of action that are similar or unrelated to those of the first and second agents and/or each other. Further, any of the agents mentioned herein can exert their biological effects by a multiplicity of mechanisms of action. The compositions, kits, and methods of the invention are described further, as follows, after a brief discussion of Alzheimer's and other amyloid-β related diseases.

Pharmacologic Treatment of Alzheimer's Disease and Other Amyloid-β Diseases

Local deposition of amyloid is common in the brain, particularly in elderly individuals. The most common type of amyloid in the brain is composed primarily of Aβ peptide fibrils, resulting in dementia associated with sporadic (non-hereditary) Alzheimer's disease. In fact, the incidence of sporadic Alzheimer's disease greatly exceeds forms shown to be hereditary Nevertheless, fibril peptides forming plaques are very similar in both types.

Amyloid precursor protein (APP) is expressed and constitutively catabolized in most cells. The dominant catabolic pathway appears to be cleavage of APP within the Aβ sequence by an enzyme provisionally termed α-secretase, leading to release of a soluble ectodomain fragment known as APPsα. In contrast to this non-amyloidogenic pathway, APP can also be cleaved by enzymes known as β and γ-secretase at the N- and C-termini of the Aβ, respectively, followed by release of Aβ into the extracellular space. To date, BACE has been identified as β-secretase (Vasser et al., Science 286, 735-741 (1999)) and presenilins have been implicated in γ-secretase activity (De Strooper et al., Nature 391, 387-390 (1998)).

The 39-43 amino acid Aβ peptide is produced by sequential proteolytic cleavage of the amyloid precursor protein (APP) by the enzyme(s) β and γ secretases. Although Aβ-40 is the predominant form produced, 5-7% of total Aβ exists as Aβ-42 (Cappai et al., Int. J. Biochem. Cell Biol. 31, 885-889 (1999)). The length of the Aβ peptide appears to dramatically alter its biochemical/biophysical properties. Specifically, the additional two amino acids at the C-terminus of Aβ-42 are very hydrophobic, presumably increasing the propensity of Aβ-42 to aggregate. For example, Jarrett et al. demonstrated that Aβ-42 aggregates very rapidly in vitro as compared to Aβ-40, suggesting that the longer forms of Aβmay be the important pathological proteins that are involved in the initial seeding of the neuritic plaques in Alzheimer's disease (Jarrett et al., Biochemistry 32,4693-4697 (1993); Jarrett et al., Ann. NY Acad. Sci. 695, 144-148, (1993)).

This hypothesis has been further substantiated by the recent analysis of the contributions of specific forms of Aβ in cases of genetic familial forms of Alzheimer's disease (FAD). For example, the “London” mutant form of APP (APPV7171) linked to FAD selectively increases the production of Aβ-42/43 forms versus Aβ-40 (Suzuki et al., Science 264, 1336-1340 (1994)), while the “Swedish” mutant form of APP (APPK670N/M671L) increases levels of both Aβ-40 and Aβ-42/43 (Citron et al., Nature 360, 672-674 (1992); Cai et al., Science 259, 514-516 (1993)). Also, it has been observed that FAD-linked mutations in the presenilin-1 (PS1) or presenilin-2 (PS2) genes will lead to a selective increase in Aβ-42/43 production but not Aβ-40 (Borchelt et al., Neuron 17, 1005-1013 (1996)). This finding was corroborated in transgenic mouse models expressing PS mutants that demonstrate a selective increase in brain Aβ-42 (Borchelt et al., Neuron 17, 1005-1013 (1996); Duff et al., Neurodegeneration 5(4), 293-298 (1996)). Thus, the leading hypothesis regarding the etiology of Alzheimer's disease is that an increase in Aβ-42 production or release is a causative event in the disease pathology.

In addition to a relationship with coronary disease, a relationship exists between serum cholesterol levels and the incidence and the pathophysiology of Alzheimer's disease. Epidemiological studies show that subjects with elevated cholesterol levels have an increased risk of Alzheimer's disease (Notkola et al., Neuroepidemiology 17(1), 14-20 (1998); Jarvik et al., Neurology 45(6), 1092-1096 (1995)). In addition to the data that suggest that elevated levels of Aβ are associated with Alzheimer's disease, other environmental and genetic risk factors have been identified. The best studied of these is polymorphism of the apolipoprotein E (ApoE) gene: subjects homozygous for the ε4 isoform of ApoE (apoE4) have consistently been shown to have an increased risk for Alzheimer's disease (Strittmatter et al., Proc. Natl. Acad. Sci. U.S.A. 90, 1977-1981 (1993). Because ApoE is a cholesterol transport protein, several groups have observed a correlation between the risk of developing Alzheimer's disease and circulating levels of cholesterol (Mahley, Science 240, 622-630 (1998), Saunders et al., Neurology 43, 1467-1472 (1993); Corder et al., Science 261, 921-923 (1993); Jarvik et al., Annals of the New York Academy of Sciences 826,128-146 (1997)). Moreover, cholesterol loading increases the production of Aβ protein (Simons et al., Proc. Natl. Acad. Sci. U.S.A. 95, 6460-6464 (1998)), while pharmacological reduction of cholesterol with the HMG CoA reductase inhibitor simvastatin decreases levels of both Aβ-40 and Aβ-42 (Fassbender et al., Proc. Natl. Acad. Sci, U.S.A. 98, 5856-5861 (2001)) in vitro. Consistent with these data are the results of epidemiological studies which have shown that treatment with certain HMG CoA reductase inhibitors, commonly used to normalize cholesterol levels in humans, reduces the prevalence of Alzheimer's disease (Wolozin et al., Arch. Neurol. 57, 1439-1443 (2000); Jick et al., Lancet 356, 1627-1631 (2000). Taken together, these data suggest a link between regulation of cholesterol levels and Alzheimer's disease.

As is discussed above, amyloid-β peptide (Aβ) is a 39-43 amino acid peptide derived by proteolysis from a large protein, known as Beta Amyloid Precursor Protein (βAPP). Mutations in βAPP result in familial forms of Alzheimer's disease, Down's syndrome, cerebral amyloid angiopathy, and senile dementia, characterized by cerebral deposition of plaques composed of Aβ fibrils and other components, which are described in further detail below. Known mutations in APP associated with Alzheimer's disease occur proximate to the cleavage sites of β or gamma-secretase, or within Aβ. For example, position 717 is proximate to the site of gamma-secretase cleavage of APP in its processing to Aβ, and positions 670/671 are proximate to the site of β-secretase cleavage. Mutations at any of these residues may result in Alzheimer's disease, presumably by causing an increase in the amount of the 42/43 amino acid form of Aβ generated from APP. The familial form of Alzheimer's disease represents only 10% of the subject population. Most occurrences of Alzheimer's disease are sporadic cases where APP and Aβ do not possess any mutation.

The structure and sequence of Aβ peptides of various lengths are well known in the art. Such peptides can be made according to methods known in the art, or-extracted from the brain according to known methods (e.g., Glenner et al., Biochem. Biophys. Res. Comm. 129, 885-890 (1984); Glenner et al., Biochem. Biophys. Res. Comm. 122, 1131-1135 (1984)). In addition, various forms of the peptides are commercially available.

The present invention relates to combination methods for treating and/or preventing amyloid-β related diseases by administering at least two agents, each of which is a compound that exerts a therapeutic effect and is useful in treating or preventing a neurological or psychological condition or disease. The first compound used in the methods of the invention is selected from alkylsulfonic acids that are useful for treating or preventing an amyloid-β related disease. Additional details and examples of such compounds are provided below. The second compound is a peptide or peptidomimetic that functions in the treatment and/or prevention of an amyloid-β related disease by inhibiting amyloid-β fibril formation and/or by inducing an immune response to amyloid-β that is protective and/or therapeutic against the disease, or an immune system modulator that functions in this manner. Further, as is noted above, therapeutic agents in addition to -the first and second agents noted above can be used in the invention in combination with the first and second agents. Examples of these additional types of agents are provided further below.

“Amyloidosis” or “amyloid disease” refers to a pathological condition characterized by the presence of amyloid fibers. Amyloid is a generic term referring to a group of diverse but specific protein deposits (intracellular or extracellular) that are seen in a number of different diseases. Though diverse in their occurrence, all amyloid deposits have common morphologic properties, stain with specific dyes (e.g., Congo red), and have a characteristic red-green birefringent appearance in polarized light after staining. They also share common ultrastructural features and common X-ray diffraction and infrared spectra. “Amyloid-β diseases” include those diseases, conditions, pathologies, and other abnormalities of the structure or function of the brain, including components thereof, in which the causative agent is amyloid. The area of the brain affected in an amyloid-β disease may be the stroma including the vasculature, the parenchyma including functional or anatomical regions, or neurons themselves. A subject need not have received a definitive diagnosis of a specifically recognized amyloid-β disease.

As used herein, the term “β amyloid” or “amyloid-β” refer to amyloid β proteins or peptides, amyloid β precursor proteins or peptides, intermediates, and modifications and fragments thereof, unless otherwise specifically indicated. In particular, “Aβ” refers to any peptide produced by proteolytic processing of the APP gene product or by synthetic or genetic means, especially peptides that are associated with amyloid pathologies, including Aβ1-39, Aβ1-40, Aβ1-41, Aβ1-42, and Aβ1-43. For convenience of nomenclature, “Aβ1-42” may be referred to herein as “Aβ(1-42)” or simply as “Aβ42” or “Aβ₄₂” (and likewise for any other amyloid peptides discussed herein). As used herein, the terms “β amyloid,” “amyloid-β,” and “Aβ” are synonymous. Unless otherwise specified, the term “amyloid” refers to amyloidogenic proteins, peptides, or fragments thereof which can be soluble (e.g., monomeric or oligomeric) or insoluble (e.g., having fibrillary structure or in amyloid plaque; see, e.g., Lambert et al., Proc. Natl. Acad. Sci. U.S.A. 95, 6448-6453 (1998)).

The term “combination” as in the phrase “a first agent in combination with a second agent” includes co-administration of a first agent and a second agent, which for example may be dissolved or intermixed in the same pharmaceutically acceptable carrier, or administration of a first agent, followed by the second agent, or administration of the second agent, followed by the first agent. The present invention, therefore, relates to methods of combination therapeutic treatment and combination pharmaceutical compositions.

The term “concomitant” as in the phrase “concomitant therapeutic treatment” includes administering an agent in the presence of a second agent. A concomitant therapeutic treatment method includes methods in which the first, second, third, or additional agents are co-administered. A concomitant therapeutic treatment method also includes methods in which the first or additional agents are administered in the presence of a second or additional agents, wherein the second or additional agents, for example, may have been previously administered. A concomitant therapeutic treatment method may be executed step-wise by different actors. For example, one actor may administer to a subject a first agent and a second actor may administer to the subject a second agent, and the administering steps may be executed at the same time, or nearly the same time, or at distant times, so long as the first agent (and additional agents) are administered in the presence of the second agent (and additional agents). The actor and the subject may be the same entity (e.g., a human).

The combination of agents used within the methods and pharmaceutical compositions described herein may have a therapeutic additive or synergistic effect on the condition(s) or disease(s) targeted for treatment. The combination of agents used within the methods or pharmaceutical compositions described herein also may reduce a detrimental effect associated with at least one of the agents when administered alone or without the other agent(s) of the particular pharmaceutical composition. For example, the toxicity of side effects of one agent may be attenuated by another agent of the composition, thus allowing a higher dosage, improving patient compliance, and improving therapeutic outcome. The additive or synergistic effects, benefits, and advantages of the compositions apply to classes of therapeutic agents, either structural or functional classes, or to individual compounds themselves.

Advantageous pharmaceutical compositions of the invention are formulated to be orally administered to a subject. The first agent and the second agent can be simultaneously administered (e.g., in a common formulation) or administered separately, optionally by different modes. The first agent and the second agent can modulate the same or different biological processes in the pathogenesis of an Aβ-related disease such as Alzheimer's disease, and act on the same or different targets. Preferably, the first agent and the second agent, when simultaneously present in a subject, act synergistically to reduce, inhibit, or ameliorate the symptoms or pathogenesis of an Aβ-related disease.

The term “subject” includes living organisms in which amyloidosis can occur. Examples of subjects that can be treated according to the present invention include humans, monkeys, cows, sheep, goats, dogs, cats, mice, rats, and transgenic species thereof Treatment of a subject using the methods, compositions, and kits of the present invention can be carried out using known procedures, at dosages and for periods of time effective to modulate amyloid aggregation in the subject as described further herein. An effective amount of a therapeutic compound necessary to achieve a therapeutic effect may vary according to factors such as the amount of amyloid already deposited at the clinical site in the subject; the age, sex, and weight of the subject; the use of concurrent therapies; and the ability of the therapeutic compound to modulate amyloid aggregation in the subject. Dosage regimens can be adjusted to provide the optimum therapeutic response. For example, several divided doses can be administered daily or a dose can be proportionally reduced as indicated by the exigencies of the therapeutic situation.

In an exemplary aspect of the invention, the subject is a human. For example, the subject can be a human over 40 years old, a human over 50 years old, a human over 60 years old, or a human over 70 years old. The subject can be a female human, including a postmenopausal female human, who may be on hormone (estrogen) replacement therapy. The subject can also be a male human.

A subject can be a human at risk for Alzheimer's disease, e.g., a human over the age of 40 or having a predisposition for Alzheimer's disease. Alzheimer's disease predisposing factors identified or proposed in the scientific literature include, among others, a genotype predisposing a subject to Alzheimer's disease; environmental factors predisposing a subject to Alzheimer's disease; past history of infection by viral and bacterial agents predisposing a subject to Alzheimer's disease; and vascular factors predisposing a subject to Alzheimer's disease. A subject can also have one or more risk factors for cardiovascular disease (e.g., atherosclerosis of the coronary arteries, angina pectoris, or myocardial infarction) or cerebrovascular disease (e.g., atherosclerosis of the intracranial or extracranial arteries, stroke, syncope, or transient ischemic attacks), such, as hypercholesterolemia, hypertension, diabetes, cigarette smoking, familial or previous history of coronary artery disease, cerebrovascular disease, or cardiovascular disease. Hypercholesterolemia typically is defined as a serum total cholesterol concentration of greater than about 5.2 mmol/L (about 200 mg/dL).

As is noted above, several genotypes are believed to predispose a subject to Alzheimer's disease. These include the genotypes such as presenilin-1, presenilin-2, and amyloid precursor protein (APP) missense mutations associated with familial Alzheimer's disease, and the genotypes apoE4 and α-2-macroglobulin, both of which are thought to increase the risk of acquiring sporadic (late-onset) Alzheimer's disease. Environmental factors have also been proposed as predisposing a subject to Alzheimer's disease, including exposure to aluminum, although the epidemiological evidence supporting this proposal is ambiguous. In addition, prior infection by certain viral or bacterial agents may predispose a subject to Alzheimer's disease, including the herpes simplex virus and Chlamydia pneumoniae. Finally, other predisposing factors for Alzheimer's disease can include risk factors for cardiovascular or cerebrovascular disease, including cigarette smoking, hypertension, and diabetes. “At risk for Alzheimer's disease” also encompasses any other predisposing factors not listed above or as yet identified and includes an increased risk for Alzheimer's disease caused by head injury, medications, diet, or lifestyle.

The methods of the present invention can be used for one or more of the following: to prevent Alzheimer's disease, to treat Alzheimer's disease, or to ameliorate symptoms of Alzheimer's disease, or to regulate production of or levels of amyloid-β (Aβ) peptides in the bloodstream or brain of a subject. Amyloid-β related diseases other than Alzheimer's disease that can be treated according to the invention are described further below.

In one embodiment, a human treated according to the invention carries one or more mutations in the genes that encode p-amyloid precursor protein, presenilin-1, or presenilin-2. In another embodiment, the human carries the Apolipoprotein E4 gene. In another embodiment, the human has a family history of Alzheimer's disease or dementia illness. In another embodiment, the human has trisomy 21 (Down's Syndrome).

In another embodiment, the subject has mild cognitive impairment (MCI), which is a condition characterized by a state of mild but measurable impairment in thinking skills, but is not necessarily associated with the presence of dementia. MCI frequently, but not necessarily, precedes Alzheimer's disease. It is a diagnosis that has most often been associated with mild memory problems, but it can also be characterized by mild impairments in other thinking skills, such as language or planning skills. However, in general, an individual with MCI will have more significant memory lapses than would be expected for someone of their age or educational background. As the condition progresses, a physician may change the diagnosis to Mild-to-Moderate Cognition Impairment, as is well understood in this art.

In another embodiment, the subject has a normal or low serum total blood cholesterol level. In another embodiment, the serum total blood cholesterol level is less than about 200 mg/dl, more preferably less than about 180, and can range from about 150 to about 200 mg/dl. In another embodiment, the total LDL cholesterol level is less than about 100 mg/dl, more preferably less than about 90 mg/dl and can range from about 30 to about 100 mg/dl. Methods of measuring serum total blood cholesterol and total LDL cholesterol are well known to those skilled in the art and, for example, include those described in International Patent Application Publication WO 99/38498 at page 11, which is incorporated herein-by reference. Methods of determining levels of other sterols in serum are disclosed in Gylling et al., J. Lipid Res. 40, 593-600 (1999), which is also incorporated herein by reference.

In another embodiment, the subject has an elevated serum total blood cholesterol level. In another embodiment, the serum total cholesterol level is at least about 200 mg/dl, more preferably at least about 220 mg/dl, and can range from about 200 to about 1000 mg/dl. In another alternative embodiment, the subject has an elevated total LDL cholesterol level. In another embodiment, the total LDL cholesterol level is greater than about 100 mg/dl, more preferably greater than about 110 mg/dl, and can range from about 100 to about 1000 mg/dl.

In another embodiment, the human is at least about 40 years of age. In another embodiment, the human is at least about 60 years of age. In another embodiment, the human is at least about 70 years of age. Preferably the human is between about 60 and 100 years of age.

In another embodiment, the subject exhibits no symptoms of Alzheimer's disease. In another embodiment, the subject is a human who is at least 40 years of age and exhibits no symptoms of Alzheimer's disease. In another embodiment, the subject is a human who is at least 40 years of age and exhibits one or more symptoms of Alzheimer's disease.

By using the methods of the present invention, the levels of amyloid β (Aβ) peptides in a subject's brain or blood can be reduced from levels prior to treatment from about 10 to about 100 percent, and preferably about 50 to about 100 percent.

In another embodiment, the subject can have an elevated level of amyloid Aβ-40 and Aβ-42 peptide in the blood prior to treatment according to the present methods of greater than about 10 pg/ml, e.g., greater than about 35 pg/ml or greater than about 40 pg/ml. In another embodiment, the elevated level of amyloid Aβ-42 peptide can range from about 30 pg/ml to about 20 pg/ml. Those of skill in the art would understand that as the Alzheimer's disease progresses, the measurable levels of amyloid β peptide (Aβ₄₂/Aβ₄₀) will present an Aβ ratio which may decrease slightly from elevated levels present before onset of the disease.

In another embodiment, the subject can have an elevated level of amyloid Aβ-40 peptide in the blood and CSF prior to treatment according to the present methods of greater than about 5 pg/ml, e.g., greater than about 50 pg/ml or greater than about 400 pg/ml. In another embodiment, the elevated level of amyloid Aβ-40 peptide can range from about 200 pg/ml to about 800 pg/ml.

In another embodiment, the subject can have an elevated level of amyloid Aβ-42 peptide in the CSF prior to treatment according to the present methods of greater than about 10 pg/ml, e.g., greater than about 200 pg/ml greater than about 500 pg/ml. In another embodiment, the level of amyloid-β peptide can range from about 10 pg/ml to about 1,000 pg/ml, eg., about 100 pg/ml to about 1,000 pg/ml.

In another embodiment, the subject can have an elevated level of amyloid Aβ-40 peptide in the CSF prior to treatment according to the present methods of greater than about 10 pg/ml, e.g., greater than about 50 pg/ml or greater than about 100 pg/ml. In another embodiment, the level of amyloid P peptide can range from about 10 pg/ml to about 1,000 pg/ml.

The amount of amyloid-β (Aβ) peptide in the brain,.CSF, or blood of a subject can be evaluated by enzyme-linked immunosorbent assay (ELISA) or quantitative immunoblotting test methods, or by quantitative SELDI-TOF, which are methods that are well known to those skilled in the art, such as is described by Zhang et al., J. Biol. Chem. 274, 8966-8972 (1999) and Zhang et al., Biochemistry 40, 5049-5055 (2001). These tests are performed on samples of the brain, CSF, or blood that have been prepared in a manner well known to those skilled in the art, for example, as is disclosed in the Example below. Another example of a useful method for measuring levels of amyloid-β peptides is by the Europium immunoassay (EIA), such as is described in WO 99/38498 at page 11, which is incorporated herein by reference.

In another embodiment, the amount of total ApoE in the bloodstream or brain of a subject can be reduced from levels prior to treatment by about 5 to about 75 percent, and preferably about 5 to about 50 percent. The amount of total ApoE can be measured in a manner well known to those skilled in the art, for example using an ELISA test kit such as Apo-Tek ApoE test kit that is available from Organon Teknica.

The methods of the invention can be applied as a therapy for a subject having Alzheimer's disease or a dementia, or the methods of the invention can be applied as a prophylaxis against Alzheimer's disease or dementia for subject with such a predisposition, as in a subject, e.g., with a genomic mutation in the APP gene, the ApoE gene, or a presenilin gene. The subject can have (or may be predisposed to developing or may be suspected of having) vascular dementia, senile dementia, mild cognitive impairment (MCI), or mild-to-moderate cognitive impairment. In addition to Alzheimer's disease, the subject can have another amyloid-β related disease such as cerebral amyloid angiopathy, or the subject may have amyloid deposits, especially amyloid-β amyloid deposits, in their brain.

Definition of Dementia

The essential features of a dementia are multiple cognitive deficits that include memory impairment and at least one of the following: aphasia, apraxia, agnosia, or a disturbance in executive functioning (the ability to think abstractly and to plan, initiate, sequence, monitor, and stop complex behavior). The order of onset and relative prominence of the cognitive disturbances and associated symptoms vary with the specific type of dementia, as discussed in the following.

Memory impairment is generally a prominent early symptom. Individuals with dementia have difficulty learning new material and may lose valuables, such as wallets and keys, or forget food cooking on the stove. In more severe dementia, individuals also forget previously learned material, including the names of loved ones. Individuals with dementia may have difficulty with spatial tasks, such as navigating around the house or in the immediate neighborhood (where difficulties with memory are unlikely to play a role). Poor judgment and poor insight are common as well. Individuals may exhibit little or no awareness of memory loss or other cognitive abnormalities. They may make unrealistic assessments of their abilities and make plans that are not congruent with their deficits and prognosis (e.g., planning to start a new business). They may underestimate the risks involved in activities (e.g., driving).

In order to make a diagnosis of dementia, the cognitive deficits must be sufficiently severe to cause impairment in occupational or social functioning and must represent a decline from a previous level of functioning. The nature and degree of impairment are variable and often depend on the particular social setting of the individual. For example, mild cognitive impairment may significantly impair an individual's ability to perform a complex job but not a less demanding one.

Cognitive or degenerative brain disorders are characterized clinically by progressive loss of memory, cognition, reasoning, judgment, and emotional stability that gradually leads to profound mental deterioration and ultimately death. It is generally believed that the disease begins a number of years before it manifests itself in the mild cognitive changes that are the early signs of Alzheimer's disease. “Dementia of the Alzheimer's Type” begins gradually, and is usually diagnosed after other specific causes have been ruled out. Diagnostic criteria for Dementia of the Alzheimer's Type include the development of multiple cognitive deficits manifested by both memory impairment (anterograde or retrograde, i.e., impaired ability to learn new information or to recall previously learned information); and one or more of the following cognitive disturbances: aphasia (language disturbance), apraxia (impaired ability to carry out motor activities despite intact motor function), agnosia (failure to recognize or identify objects despite intact sensory function), disturbance in executive functioning (i.e., planning, organizing, sequencing, and abstracting); where these cognitive deficits each cause significant impairment in social or occupational functioning and represent a significant decline from a previous level of functioning. The course is characterized by gradual onset and continuing cognitive decline, and the cognitive deficits are not due to another condition that causes progressive deficits in memory and cognition (e.g., cerebrovascular disease, brain tumor, hypothyroidism, vitamin B or folic acid deficiency, niacin deficiency, hypercalcemia, neurosyphilis, HIV infection, or chemical exposre). The cognitive disturbance can be accompanied by a behavioral disturbance, such as wandering, aggression, or agitation, or a psychological disturbance, such as depression or psychosis. See “Diagnostic and Statistical Manual of Mental Disorders,” 4^{th} Ed., Text Revision, by American Psychiatric Association (2000). For example, the National Institute of Neurological and Communicative Disorders and Stroke-Alzheimer's Disease and the Alzheimer's Disease and Related Disorders Association (NINCDS-ADRDA) criteria can be used to diagnose Alzheimer's Disease (McKhann et al., Neurology 34, 939-944 (1984)). The patient's cognitive function can be assessed by the Alzheimer's Disease-Assessment Scale-cognitive subscale (ADAS-cog; Rosen et al., Am J. Psychiatry 141, 1356-1364 (1984)).

The study of the pathobiology of Alzheimer's disease has identified at least four chromosomal loci associated with familial cases; the degeneration of central neurochemical systems, especially basal forebrain structures related to acetylcholine-mediated neurotransmission; factors associated with the formation of plaques and tangles; and exogenous (e:g., infectious and toxic) processes that may contribute to the development of sporadic cases. Although amyloid itself is a normal brain product, it has been suggested that excessive amounts may be neurotoxic.

For dementia of the Alzheimer's type, a family history of the dementia is probably the most important risk factor after advanced age. A family history of Down's syndrome or of hematological malignancies, such as leukemia, myelolymphoma, or Hodgkin's disease, is also associated with an increased risk for Alzheimer's disease. Degenerative dementias as a group do not have well-established risk factors other than old age and familial patterns. For dementia of the Alzheimer's type, other risk factors identified tentatively in recent years include female sex, a past history of head trauma, and lower education. Vascular dementias are highly associated with the risk factors for cerebrovascular disease. Those factors include hypertension (especially with systolic pressures greater than 160 mmHg), cardiac disease, transient ischemic attacks, diabetes mellitus, carotid bruits, and sickle cell disease. Obesity, a sedentary lifestyle, tobacco use, alcohol consumption, and elevated serum cholesterol and lipid levels are less well established as risk factors for cerebrovascular disease.

The course and prognosis of a dementia syndrome vary with its cause. Dementia does not necessarily equal progressive deterioration, although many of the pathobiological processes underlying dementia are degenerative. The rate of progression may vary within families or from individual to individual. Age at onset is an important feature of Alzheimer's disease, the most common cause of dementia in the United States. Onset usually occurs after age 60 years and the prevalence increases exponentially with each successive decade, although cases have been reported in patients as young as 30 years. Familial forms of dementia of the Alzheimer's type appear to have an earlier age at onset. Cerebrovascular disease, the second most common cause of dementia, is associated with an earlier age at onset overall.

As used herein, “treatment” of a subject includes the application or administration of a composition or the components of a kit of the invention to a subject, or application or administration of a composition of the invention to a cell or tissue from a subject, who has an amyloid-β related disease or condition, has a symptom of such a disease or condition, or is at risk of (or susceptible to) such a disease or condition, with the purpose of curing, healing, alleviating, relieving, altering, remedying, ameliorating, improving, or affecting the disease or condition, the symptom of the disease or condition, or the risk of (or susceptibility to) the disease or condition. The term “treating” refers to any indicia of success in the treatment or amelioration of an injury, pathology or condition, including any objective or subjective parameter such as abatement; remission; diminishing of symptoms or making the injury, pathology or condition more tolerable to the subject; slowing in the rate of degeneration or decline; making the final point of degeneration less debilitating; improving a subject's physical or mental well-being; or, in some situations, preventing the onset of dementia. The treatment or amelioration of symptoms can be based on objective or subjective parameters, including the results of a physical examination or a psychiatric evaluation. For example, the methods of the invention can successfully treat a subject's dementia by slowing the rate or lessening the extent of cognitive decline.

Also, the invention relates to methods for preventing or inhibiting amyloid deposition in a subject. For example, such a method comprises administering to a subject a therapeutically effective amount of a pharmaceutical composition of the invention capable of reducing the concentration of Aβ, such that amyloid production or accumulation is prevented or inhibited.

In another aspect, the invention relates to a method in which at least the first compound is for preventing, reducing, or inhibiting amyloid deposition in a subject. For example, such methods can include administering to a subject a therapeutically effective amount of a pharmaceutical composition capable of inhibiting Aβ accumulation, such that Aβ amyloidosis is prevented, reduced, or inhibited

“Inhibition” of amyloid deposition includes the preventing or stopping of amyloid formation, e.g., fibrillogenesis, inhibiting or slowing down of further amyloid deposition in a subject with amyloidosis, e.g., already having amyloid deposits, and reducing or reversing amyloid fibrillogenesis or deposits in a subject with ongoing amyloidosis. Inhibition of amyloid deposition is determined relative to an untreated subject, or relative to the treated subject prior to treatment, or, e.g., determined by clinically measurable improvement, or in the case of a subject with brain amyloidosis, e.g., an Alzheimer's or cerebral amyloid angiopathy subject, stabilization of cognitive function or prevention of a further decrease in cognitive function (Le., preventing, slowing, or stopping disease progression), or improvement of parameters such as the concentration of Aβ or tau in the CSF.

“modulation” of amyloid deposition includes both inhibition, as defined above, and enhancement of amyloid deposition or fibril formation. The term “modulating” is intended, therefore, to encompass (i) prevention or stopping of amyloid formation or accumulation, inhibition or slowing down of further amyloid aggregation in a subject with ongoing amyloidosis, e.g., already having amyloid aggregates, and reducing or reversing of amyloid aggregates in a subject with ongoing amyloidosis, and (ii) enhancing amyloid deposition, e.g., increasing the rate or amount of amyloid deposition in vivo or in vitro. Amyloid-enhancing compounds may be useful in animal models of amyloidosis, for example, to make possible the development of amyloid deposits in animals in a shorter period of time or to increase amyloid deposits over a selected period of time. Amyloid-enhancing compounds may be useful in screening assays for compounds that inhibit amyloidosis in vivo, for example, in animal models, cellular assays and in vitro assays for amyloidosis. Such compounds can be used, for example, to provide faster or more sensitive assays for compounds. In some cases, amyloid enhancing compounds may also be administered for therapeutic purposes, e.g., to enhance the deposition of amyloid in the lumen rather than the wall of cerebral blood vessels to prevent cerebral amyloid angiopathy (CAA). Modulation of amyloid aggregation is determined relative to an untreated subject or relative to the treated subject prior to treatment.

In an embodiment, the method is used to treat Alzheimer's disease (e.g., sporadic or familial Alzheimer's disease). The method can also be used prophylactically or therapeutically to treat other clinical occurrences of amyloid-β deposition, such as in Down's syndrome individuals and in subjects with CAA or hereditary cerebral hemorrhage. In another embodiment, the method is used to treat mild cognitive impairment, as is described above.

Additionally, abnormal accumulation of APP and amyloid-β protein in muscle fibers has been implicated in the pathology of sporadic inclusion body myositis (IBM) (Askanas et al., Proc. Natl. Acad. Sci. U.S.A. 93, 1314-1319 (1996); Askanas et al., Current Opinion in Rheumatology 7, 486-496 (1995)). Accordingly, the compounds of the invention can be used prophylactically or therapeutically in the treatment of disorders in which amyloid-β protein is abnormally deposited at non-neurological locations, such as treatment of IBM by delivery of the compounds to muscle fibers.

Additionally, it has been shown that Aβ is associated with abnormal extracellular deposits, known as drusen, that accumulate along the basal surface of the retinal pigmented epithelium in individuals with age-related macular degeneration (ARMD). ARMD is a cause of irreversible vision loss in older individuals. It is believed that Aβ deposition could be an important component of the local inflammatory events that contribute to atrophy of the retinal pigmented epithelium, drusen biogenesis, and the pathogenesis of ARMD (Johnson et al., Proc. Natl. Acad. Sci. U.S.A. 99(18),11830-11835 (2002)).

The present invention therefore relates to the use of alkylsulfonic acid compounds in the prevention or treatment of amyloid-β related diseases, including, inter alia, Alzheimer's disease, cerebral amyloid angiopathy, inclusion body myositis, Down's syndrome, and macular degeneration, in combination with a second therapeutic agent, that is a peptide or peptidomimetic that blocks fibril formation or induces a therapeutic immune response against the β-amyloid related disease or an immune system modulator that has such an effect.

In one embodiment, the pharmaceutical compositions disclosed herein prevent or inhibit amyloid protein assembly into insoluble fibrils which, in vivo, are deposited in various organs, or it reverses or favors deposition in subjects already having deposits. In another embodiment, the agent may also prevent the amyloid protein, in its soluble, oligomeric form or in its fibrillar form, from binding or adhering to a cell surface and causing cell damage or toxicity. In yet another embodiment, the agent may block amyloid-induced cellular toxicity or microglial activation. In another embodiment, the agent may block amyloid-induced neurotoxicity.

The pharmaceutical compositions of the invention may be administered therapeutically or prophylactically to treat diseases associated with amyloid-β fibril formation, aggregation or deposition. The pharmaceutical compositions of the invention may act to ameliorate the course of an amyloid-β related disease using any of the following mechanisms (this list is meant to be illustrative and not limiting): slowing the rate of amyloid-β fibril formation or deposition; lessening the degree of amyloid-β deposition; inhibiting, reducing, or preventing amyloid-β fibril formation; inhibiting neurodegeneration or cellular toxicity induced by amyloid-β; inhibiting amyloid-β induced inflammation; or enhancing the clearance of amyloid-β from the brain.

Pharmaceutical compositions of the invention may be effective in controlling amyloid-β deposition either following their entry into the brain (following penetration of the blood brain barrier) or from the periphery. When acting from the periphery, an agent of a pharmaceutical composition may alter the equilibrium of Aβ between the brain and the plasma so as to favor the exit of Aβ from the brain. An increase in the exit of Aβ from the brain would result in a decrease in Aβ brain concentration and therefore favor a decrease in Aβ deposition. Alternatively, agents that penetrate the brain could control deposition by acting directly on brain Aβ, e.g., by maintaining it in a non-fibrillar form or favoring its clearance from the brain. These agents could also prevent Aβ in the brain from interacting with a cell surface and therefore prevent neurotoxicity or inflammation.

In one embodiment, the pharmaceutical compositions disclosed herein prevent or inhibit amyloid protein assembly into insoluble fibrils which, in vivo, are deposited in various organs, or it favors plaque clearance or slows deposition in subjects already having deposits. In another embodiment, the pharmaceutical compositions may also prevent the amyloid protein, in its soluble, oligomeric form or in its fibrillar form, from binding or adhering to a cell surface and causing cell damage or toxicity. In yet another embodiment, the pharmaceutical compositions may block amyloid toxicity.

The compositions of the invention can be administered therapeutically or prophylactically to treat diseases associated with amyloid-β fibril formation, aggregation, or deposition. The compositions of the invention may act to ameliorate the course of an amyloid-β related disease using any of the following mechanisms (this list is meant to be illustrative and not limiting): slowing the rate of amyloid-β fibril formation or deposition; lessening the degree of amyloid-β deposition; inhibiting, reducing, or preventing amyloid-β fibril formation; inhibiting neurodegeneration or cellular toxicity induced by amyloid-β; inhibiting amyloid-β induced inflammation; or enhancing the clearance of amyloid-β from the brain.

At least one of the therapeutic agents of the invention may be effective in controlling amyloid-β deposition either following their entry into the brain (following penetration of the blood brain barrier) or from the periphery. When acting from the periphery, an agent may alter the equilibrium of Aβ between the brain and the plasma so as to favor the exit of Aβ from the brain. An increase in the exit of Aβ from the brain would result in a decrease in Aβ brain concentration and therefore favor a decrease in Aβ deposition. Alternatively, agents that penetrate the brain could control deposition by acting directly on brain Aβ, e.g. by maintaining it in a non-fibrillar form or favoring its clearance from the brain.

In one aspect, the invention relates to pharmaceutical compositions comprising two or more agents, each of which exerts a therapeutic effect when administered to a subject in need thereof, and is useful in treating or preventing a neurological disease. The first agent of a pharmaceutical composition of the invention is selected from alkylsulfonic acids that are useful for treating or preventing an amyloid-β related disease, as is discussed further below. Also as is discussed further below, the second agent is a peptide or peptidomimetic compound that, as is noted above, can interfere with Aβ fibril formation or induce a therapeutic immune response against Aβ, or an immune system modulator (e.g., an antibody) that is therapeutic against Aβ.

The invention also relates to packaged pharmaceutical products containing two or more agents, each of which exerts a therapeutic effect when administered to a subject in need thereof, and is useful in treating or preventing a neurological disease (e.g., Alzheimer's disease and the other amyloid-β related disease mentioned herein). The first agent of a pharmaceutical composition of the invention is selected from alkylsulfonic acids that are useful for treating or preventing an amyloid-β related disease, while the second agent is apeptide, a peptidomimetic compound, or an immune system modulator (e.g., an antibody), as is described above and elsewhere herein.

In some cases, the individual agents can be packaged in separate containers for sale or delivery to the consumer. The agents of the invention can be supplied in a solution with an appropriate solvent or in a solvent-free form (e.g., lyophilized). Additional components can include acids, bases, buffering agents, inorganic salts, solvents, antioxidants, preservatives, or metal chelators, as well as additional therapeutic, as is mentioned above. The additional kit components are present as pure compositions, or as aqueous or organic solutions that incorporate one or more additional kit components. Any or all of the kit components optionally further comprise buffers.

Since one aspect of the present invention relates to treating Alzheimer's disease or regulating production of or levels of amyloid-β (Aβ) peptides in the bloodstream or brain by treatment with a combination of active ingredients, wherein the active ingredients can be administered separately, the invention also relates to combining separate pharmaceutical compositions in kit form. That is, a kit is contemplated in which two separate units are combined: a pharmaceutical composition comprising at least one alkylsulfonic acid compound (see below) and a separate pharmaceutical composition comprising a peptide, peptidomimetic compound, or immune system modulator, as is discussed above. The kit may also include directions for the administration of the components. The kit form is particularly advantageous when the separate components must be administered in different dosage forms (e.g., oral and parenteral) or are administered at different dosage intervals.

The present invention also includes packaged pharmaceutical products containing a first agent in combination with (e.g., intermixed with) a second agent. The invention also includes a pharmaceutical product comprising a first agent packaged with instructions for using the first agent in the presence of a second agent or instructions for use of the first agent in a method of the invention. The invention also includes a pharmaceutical product comprising a second or additional agents packaged with instructions for using the second or additional agents in the presence of a first agent or instructions for use of the second or additional agents in a method of the invention. Alternatively, the packaged pharmaceutical product may contain at least one of the agents and the product may be promoted for use with a second agent.

The term “agonist,” as used herein, refers to a molecule which, when interacting with a biologically active molecule, causes a change (e.g., enhancement) in the biologically active molecule, which positively modulates the activity of the biologically active molecule. Agonists include, but are not limited to proteins, nucleic acids, carbohydrates, lipids, or any other molecules that bind or interact with biologically active molecules. For example, agonists can alter the activity of gene transcription by interacting with RNA polymerase directly or through a transcription factor or signal transduction pathway. The terms “antagonist” or “inhibitor,” as used herein, refer to a molecule which, when interacting with a biologically active molecule, blocks or negatively modulates the biological activity of the biologically active molecule. Antagonists and inhibitors include, but are not limited to, proteins, nucleic acids, carbohydrates, lipids, or any other molecules that bind or interact with biologically active molecules. Inhibitors and antagonists can affect the biology of entire cells, organs, or organisms (e.g., an inhibitor that slows or prevents neuronal degeneration and death).

Blood-Brain Barrier

Agents of the invention that exert their physiological effect in vivo in the brain may be more useful if they gain access to target cells in the brain. Non-limiting examples of brain cells are neurons, glial cells (e.g., astrocytes, oligodendrocytes, and microglia), cerebrovascular cells (e.g., muscle cells and endothelial cells), and cells that comprise the meninges. The blood brain barrier (“BBB”) typically restricts access to brain cells by acting as a physical and functional blockade that separates the brain parenchyma from the systemic circulation (see, e.g., Pardridge et al., J. Neurovirol. 5(6), 556-569 (1999); Rubin et al., Rev. Neurosci. 22, 11-28 (1999)). Circulating molecules are normally able to gain access to brain cells via one of two processes: lipid-mediated transport through the BBB by free diffusion, or active (or catalyzed) transport.

The agents of the invention may be formulated to improve distribution in vivo, for example as powdered or liquid tablet or solution for oral administration or as a nasal spray, nose drops, a gel or ointment, through a tube or catheter, by syringe, by packtail, by pledget, or by submucosal infusion. For example, the blood-brain barrier (BBB) excludes many highly hydrophilic agents. To ensure that the more hydrophilic therapeutic agents of the invention cross the BBB, they may be formulated, for example, in liposomes. For methods of manufacturing liposomes, see, e.g., U.S. Pat. Nos. 4,522,811; 5,374,548; and 5,399,331. The liposomes may comprise one or more moieties which are selectively transported into specific cells or organs (“targeting moieties,” “targeting groups,” or “transporting vectors”), thus providing targeted drug delivery (see, e.g., Ranade, J. Clin. Pharmacol. 29, 685 (1989)). Likewise, the agents maybe linked to targeting groups that facilitate penetration of the blood brain barrier. In one embodiment, the methods of the present invention employ a naturally occurring polyamine linked to an agent of the invention and is useful for inhibiting Aβ deposition.

Natural cell metabolites that may be used as targeting groups; include, inter alia, putrescine, spermidine, spermine, or DHA. Other exemplary targeting moieties include folate or biotin (see, e.g., U.S. Pat. No.5,416,016); mannosides (Umezawa et al., Biochem. Biophys. Res. Commun. 153, 1038 (1988)); antibodies (Bloeman et al., FEBS Lett. 357, 140 (1995); Owais et al., Antimicrob. Agents Chemother. 39, 180 (1995)); surfactant protein A receptor (Briscoe et al., Am. J. Physiol. 1233, 134 (1995)); gp120 (Schreier et al., J. Biol. Chem. 269, 9090 (1994); see also, Keinanen et al., FEBS Lett. 346, 123 (1994); and Killion et al., Immunomethods 4, 273 (1994)).

To facilitate transport of agents of the invention across the BBB, they may be coupled to a BBB transport vector (for review of BBB transport vectors and mechanisms, see, Bickel et al., Adv. Drug Delivery Reviews 46, 247-279 (2001)). Exemplary transport vectors include cationized albumin or the OX26 monoclonal antibody to the transferrin receptor, these proteins undergo absorptive-mediated and receptor-mediated transcytosis through the BBB, respectively.

Examples of other BBB transport vectors that target receptor-mediated transport systems into the brain include factors such as insulin, insulin-like growth factors (“IGF-I” and “IGF-II”), angiotensin II, atrial and brain natriuretic peptide (“ANP” and “BNP”), interleukin I (“IL-1”) and transferrin. Monoclonal antibodies to the receptors that bind these factors may also be used as BBB transport vectors. BBB transport vectors targeting mechanisms for absorptive-mediated transcytosis include cationic moieties such as cationized LDL, albumin, or horseradish peroxidase coupled with polylysine, cationized albumin, or cationized immunoglobulins. Small basic oligopeptides such as the dynorphin analogue E-2078 and the ACTH analogue ebiratide may also cross the brain via absorptive-mediated transcytosis and are potential transport vectors.

Other BBB transport vectors target systems for transporting nutrients into the brain. Examples of such BBB transport vectors include hexose moieties, e.g. glucose, and monocarboxylic acids, e.g., lactic acid, and neutral amino acids, e.g., phenylalanine, and amines, e.g., choline, and basic amino acids, e.g., arginine, nucleosides, e.g. adenosine, and purine bases, e.g., adenine, and thyroid hormone, e.g., triiodothyridine. Antibodies to the extracellular domain of nutrient transporters may also be used as transport vectors. Other possible vectors include angiotensin 11 and ANP, which may be involved in regulating BBB permeability.

In some cases, the bond linking the therapeutic agent to the transport vector may be cleaved following transport into the brain in order to liberate the biologically active agent. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector.

Transcytosis, including receptor-mediated transport of compositions across the blood brain barrier, may also be suitable for the agents of the invention. Transferrin receptor-mediated delivery is disclosed in U.S. Pat. Nos. 5,672,683; 5,383,988; 5,527,527; 5,977,307; and 6,015,555. Transferrin-mediated transport is also known (Friden et al., Pharmacol. Exp. Ther. 278, 1491-98 (1996); Lee, J. Pharmacol. Exp. Ther. 292, 1048-1052 (2000)). EGF receptor-mediated delivery is disclosed in Deguchi et al., Bioconjug. Chem. 10, 32-37 (1999), and transcytosis is described in Cerletti et al., J. Drug Target. 8, 435-446 (2000). Insulin fragments have also been used as carriers for delivery across the blood brain barrier (Fukuta et al., Pharm. Res. 11, 1681-1688 (1994)). Delivery of agents via a conjugate of neutral avidin and cationized human albumin has also been described (Kang et al., Pharm. Res. 1, 1257-1264 (1994)).

Other modifications that enhance penetration of the agents of the invention across the blood brain barrier may be made using methods and derivatives known in the art. For example, U.S. Pat. No. 6,024,977 discloses covalent polar lipid conjugates for targeting to brain and central nervous system, and U.S. Pat. No.5,017,566 discloses cyclodextrin derivatives comprising inclusion complexes of lipoidal forms of dihydropyridine redox targeting moieties. U.S. Pat. No. 5,023,252 discloses the use of pharmaceutical compositions comprising a neurologically active drug and a compound for facilitating transport of the drug across the blood-brain barrier including a macrocyclic ester, diester, amide, diamide, amidine, diamidine, thioester, dithioester, thioamide, ketone, or lactone. U.S. Pat. No. 5,024,998 discloses parenteral solutions of aqueous-insoluble drugs with cyclodextrin derivatives. U.S. Pat. No. 5,039,794 discloses the use of a metastatic tumor-derived egress factor for facilitating the transport of compounds across the blood-brain barrier. U.S. Pat. No. 5,112,863 discloses the use of N-acyl amino acid derivatives as antipsychotic drugs for delivery across the blood-brain barrier. U.S. Pat. No. 5,124,146 discloses a method for delivery of therapeutic agents across the blood-brain barrier at sites of increase permeability associated with brain lesions. U.S. Pat. No. 5,153,179 discloses acylated glycerol and derivatives for use in a medicament for improved penetration of cell membranes. U.S. Pat. No. 5,177,064 discloses the use of lipoidal phosphonate derivatives of nucleoside antiviral agents for delivery across the blood-brain barrier. U.S. Pat. No. 5,254,342 discloses receptor-mediated transcytosis of the blood-brain barrier using the transferrin receptor in combination with pharmaceutical compounds that enhance or accelerate this process. U.S. Pat. No. 5,258,402 discloses treatment of epilepsy with imidate derivatives of anticonvulsive sulfamate. U.S. Pat. No. 5,270,312 discloses substituted piperazines as central nervous system agents. U.S. Pat. No. 5,284,876 discloses fatty acid conjugates of dopamine drugs. U.S. Pat. No. 5,389,623 discloses the use of lipid dihydropyridine derivatives of anti-inflammatory steroids or steroid sex hormones for delivery across the blood-brain barrier. U.S. Pat. No. 5,405,834 discloses prodrug derivatives of thyrotropin releasing hormone. U.S. Pat. No. 5,413,996 discloses acyloxyalkyl phosphonate conjugates of neurologically-active drugs for anionic sequestration of such drugs in brain tissue. U.S. Pat. No. 5,434,137 discloses methods for the selective opening of abnormal brain tissue capillaries using bradykinin infused into the carotid artery. U.S. Pat. No. 5,442,043 discloses a peptide conjugate between a peptide having a biological activity and incapable of crossing the blood-brain barrier and a peptide which exhibits no biological activity and is capable of passing the blood-brain barrier by receptor-mediated endocytosis. U.S. Pat. No. 5,466,683 discloses water soluble analogues of an anticonvulsant for the treatment of epilepsy. U.S. Pat. No. 5,525,727 discloses compositions for differential uptake and retention in brain tissue comprising a conjugate of a narcotic analgesic and agonists and antagonists thereof with a lipid form of dihydropyridine that forms a redox salt upon uptake across the blood-brain barrier that prevents partitioning back to the systemic circulation.

Still further examples of modifications that enhance penetration of the blood brain barrier are described in International (PCT) Application Publication Number WO 85/02342, which discloses a drug composition comprising a glycerolipid or derivative thereof. PCT Publication Number WO 89/11299 discloses a chemical conjugate of an antibody with an enzyme that is delivered specifically to a brain lesion site for activating a separately-administered neurologically-active prodrug. PCT Publication Number WO 91/04014 discloses methods for delivering therapeutic and diagnostic agents across the blood-brain barrier by encapsulating the drugs in liposomes targeted to brain tissue using transport-specific receptor ligands or antibodies. PCT Publication Number WO 91/04745 discloses transport across the blood-brain barrier using cell adhesion molecules and fragments thereof to increase the permeability of tight junctions in vascular endothelium. PCT Publication Number WO 91/14438 discloses the use of a modified, chimeric monoclonal antibody for facilitating transport of substances across the blood-brain barrier. PCT Publication Number WO 94/01131 discloses lipidized proteins, including antibodies. PCT Publication Number WO 94/03424 discloses the use of amino acid derivatives as drug conjugates for facilitating transport across the blood-brain barrier. PCT Publication Number WO 94/06450 discloses conjugates of neurologically-active drugs with a dihydropyridine-type redox targeting moiety and comprising an amino acid linkage and an aliphatic residue. PCT Publication Number WO 94/02178 discloses antibody-targeted liposomes for delivery across the blood-brain barrier. PCT Publication Number WO 95/07092 discloses the use of drug-growth factor conjugates for delivering drugs across the blood-brain barrier. PCT Publication Number WO 96/00537 discloses polymeric microspheres as injectable drug-delivery vehicles for delivering bioactive agents to sites within the central nervous system. PCT Publication Number WO 96/04001 discloses omega-3-fatty acid conjugates of neurologically-active drugs for brain tissue delivery. PCT WO 96/22303 discloses fatty acid and glycerolipid conjugates of neurologically-active drugs for brain tissue delivery.

In general, it is well within the ordinary skill in the art to prepare an ester, amide, or hydrazide derivative of an agent of the invention, for example, from the corresponding carboxylic acid and a suitable reagent. For instance, a carboxylic acid-containing compound, or a reactive equivalent thereof, may be reacted with a hydroxyl-containing compound, or a reactive equivalent thereof, so as to provide the corresponding ester. See, e.g. “Comprehensive Organic Transformations,” 2^{nd} Ed., by R. C. Larock, VCH Publishers John Wiley & Sons, Ltd. (1989); “March's Advanced Organic Chemistry,” 5^{th} Ed., by M. B. Smith and J. March, John Wiley & Sons, Ltd. (2000).

An alternative method to deliver an agent across the BBB is by intracerebroventricular pump or by delivery to the nasal cavity, e.g., the olfactory epithelium in the upper third of the nasal cavity, in order to promote transport of the agent into the peripheral olfactory neurons rather than the capillaries within the respiratory epithelium. In one embodiment the transport of agents to the brain is accomplished by means of the nervous system instead of the circulatory system so agents of the invention that are small molecules may be delivered to the appropriate areas of the brain. It is preferable that the agent be capable of at least partially dissolving in the fluids that are secreted by the mucous membrane that surround the cilia of the olfactory receptor cells of the olfactory epithelium in order to be absorbed into the olfactory neurons. Alternatively, the agent may be combined with a carrier or other substances that foster dissolution of the agent within nasal releases. Potential adjuvants include GM-1, phosphatidylserine, and emulsifiers such as polysorbate 80 or other additives that promote the absorption of the agent into the peripheral olfactory receptor cells. Because of their role in odor detection, these peripheral neurons provide a direct connection between the brain and the outside environment. The olfactory receptor cells are bipolar neurons with swellings covered by hair-like cilia that project into the nasal cavity. At the other end, axons from these cells collect into aggregates and enter the cranial cavity at the roof of the nose. For example, the agent may be lipophilic in order to promote absorption into the olfactory neurons and through the olfactory epithelium, or the agent may be combined with a carrier or other substances that enhance the absorption of the agent into the olfactory neurons. Among the supplementary substances that are preferred are lipophilic substances such as gangliosides and phosphatidylserine. Uptake of non-lipophilic agents may be enhanced by the combination with a lipophilic substance. In one embodiment of the method of the invention, the agent may be combined with micelles comprised of lipophilic substances. Such micelles may modify the permeability of the nasal membrane and enhance absorption of the agent. Among the lipophilic micelles that are preferred are gangliosides, particularly GM-1 ganglioside, and phosphatidylserine. The agent may be combined with one or several types of micelle substances.

Therapeutic Drug Targets for the Treatment of Alzheimer 's Disease

In the pharmaceutical compositions of the invention, an alkylsulfonic acid compound is combined with a peptide, peptidomimetic compound, or immune system modulator (e.g., antibody), as described elsewhere herein. In addition, any of the following therapeutic agents that are useful in the treatment of Alzheimer's disease, other Aβ-related diseases, and associated diseases and conditions can be used. In general, the additional therapeutic agents may be any therapeutic drug. A “therapeutic drug” is a drug or medicine administered for legitimate or medically-approved, therapeutic or diagnostic, purpose. Therapeutic drugs may be available over-the-counter or by prescription. Examples of therapeutic drugs include an adrenergic, anti-adrenergic, anti-androgen, anti-anginal, anti-anxiety, anticonvulsant, antidepressant, anti-epileptic, antihyperlipidemic, antihyperlipoproteinemic, antihypertensive, anti-inflammatory, antiobessional, antiparkinsonian, antipsychotic, adrenocortical steroids; adrenocortical suppressant; aldosterone antagonists; amino acids; anabolic steroids; analeptic agents; androgens; blood glucose regulators; cardioprotectants; cardiovascular agents; cholinergic agonist and antagonists; cholinesterase deactivators or inhibitors; cognition adjuvants and enhancers; dopaminergic agents; enzyme inhibitors; estrogen, *ee oxygen radical scavengers; GABA agonists; glutamate antagonists; hormones; hypocholesterolemic agents; hypolipidemic agents; hypotensive agents; immunizing agents; immunostimulants; monoamine oxidase inhibitor, neuroprotective agents; NMDA antagonists; AMPA antagonists, competitive and non-competitive NMDA antagonists; opioid antagonists; potassium channel openers; non-hormonal sterol derivatives; post-stroke and post-head trauma treatments; prostaglandins; psychotropics; relaxants; sedatives; sedative-hypnotics; selective adenosine antagonists; serotonin antagonists; serotonin inhibitors; selective serotonin uptake inhibitor; serotonin receptor antagonists; sodium and calcium channel blockers; steroids; stimulants; thyroid hormones and inhibitors, etc.

In one aspect, thus, the invention pertains to a pharmaceutical composition comprising an alkylsulfonic acid and a peptide, peptidomimetic compound, or an immune system modulator (e.g., an antibody), and a further agent that is useful in the treatment or prevention of Alzheimer's disease. The further agent may be curative, i.e., modulate the causative agents of Alzheimer's disease, or it may be palliative, i.e., alleviate the symptoms of the disease, e.g., by enhancing memory or improving cognitive function. The further agent may be a drug that is useful in the treatment of Alzheimer's disease itself, or it may be used to treat a condition associated with Alzheimer's disease, e.g., a secondary condition, or it may be a drug commonly prescribed to Alzheimer's disease subjects.

The language “in combination with” a second or further agent or treatment includes co-administration of an alkylsulfonic acid, administration of an alkylsulfonic acid first, followed by the second or further agent, or treatment and administration of the second or further agent first, followed by an alkylsulfonic acid.

The condition associated with Alzheimer's disease may be a symptom characteristic of Alzheimer's disease, for example, hypothyroidism, cerebrovascular or cardiovascular disease, memory loss, anxiety, or a behavioral dysfunction (e.g., apathy, aggression, or incontinence); a psychological condition or a neurological condition. The neurological condition may be Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, or posthypoxic damage. The psychological condition is depression, delusions, illusions, hallucinations, sexual disorders, weight loss, psychosis, a sleep disturbance such as insomnia, behavioral disinhibition, poor insight, suicidal ideation, depressed mood or irritability, anhedonia, social withdrawal, or excessive guilt.

Prodrugs

The present invention is also related to prodrugs of the agents of the Formulae disclosed herein. Prodrugs are agents that are converted in vivo to active forms (see, e.g. Silverman, 1992, “The Organic Chemistry of Drug Design and Drug Action,” Academic Press, Chpt. 8). Prodrugs can be used to alter the biodistribution (e.g. to allow agents which would not typically enter the reactive site of the protease) or the pharmacokinetics for a particular agent. For example, a carboxylic acid group, can be esterified, e.g., with a methyl group or an ethyl group to yield an ester. When the ester is administered to a subject, the ester is cleaved, enzymatically or non-enzymatically, reductively, oxidatively, or hydrolytically, to reveal the anionic group. An anionic group can be esterified with moieties (eg., acyloxymethyl esters) that are cleaved to reveal an intermediate agent that subsequently decomposes to yield the active agent. The prodrug moieties may be metabolized in vivo by esterases or by other mechanisms to carboxylic acids.

Examples of prodrugs and their uses are well known in the art (see, e.g. Berge et al., J. Pharm. Sci. 66, 1-19 (1977)). The prodrugs can be prepared in situ during the final isolation and purification of the agents, or by separately reacting the purified agent in its free acid form with a suitable derivatizing agent. Carboxylic acids can be converted into esters via treatment with an alcohol in the presence of a catalyst.

Examples of cleavable carboxylic acid prodrug moieties include substituted and unsubstituted, branched or unbranched lower alkyl ester moieties (eg., ethyl esters, propyl esters, butyl esters, pentyl esters, cyclopentyl esters, hexyl esters, cyclohexyl esters), lower alkenyl esters, dilower alkyl-amino lower-alkyl esters (e.g., dimethylaminoethyl ester), acylamino lower alkyl esters, acyloxy lower alkyl esters (e.g., pivaloyloxymethyl ester), aryl esters (phenyl ester), aryl-lower alkyl esters (e.g., benzyl ester), substituted (e.g. with methyl, halo, or methoxy substituents) aryl and aryl-lower alkyl esters, amides, lower-alkyl amides, dilower alkyl amides, and hydroxy amides.

Pharmaceutical Preparations

In another embodiment, the present invention relates to pharmaceutical compositions including any of the agents described herein for the treatment of an amyloid-β related disease, as well as methods of manufacturing such pharmaceutical compositions.

In general, the agents of the present invention may be prepared by the methods illustrated in the general reaction schemes as, for example, in the patents and patent applications referred to herein, or by modifications thereof, using readily available starting materials, reagents and conventional synthesis procedures. In these reactions, it is also possible to make use of variants that are in themselves known, but are not mentioned here. Functional and structural equivalents of the agents described herein and which have the same general properties, wherein one or more simple variations of substituents are made which do not adversely affect the essential nature or the utility of the agent, may also be used.

The agents of the invention may be supplied in a solution with an appropriate solvent or in a solvent-free form (e.g., lyophilized). In another aspect of the invention, the agents and buffers necessary for carrying out the methods of the invention may be packaged as a kit. The kit may be commercially used according to the methods described herein and may include instructions for use in a method of the invention. Additional kit components way include acids, bases, buffering agents, inorganic salts, solvents, antioxidants, preservatives, or metal chelators. The additional kit components are present as pure compositions, or as aqueous or organic solutions that incorporate one or more additional kit components. Any or all of the kit components optionally further comprise buffers.

The therapeutic agents may also be administered parenterally, intraperitoneally, intraspinally, or intracerebrally. Dispersions can be prepared in glycerol, liquid polyethylene glycols, and mixtures thereof and in oils. Under ordinary conditions of storage and use, these preparations may contain a preservative to prevent the growth of microorganisms.

To administer the therapeutic agents by routes other than parenteral administration, it may be necessary to coat the agent with, or co-administer the agent with, a material to prevent its inactivation. For example, the therapeutic agent may be administered to a subject in an appropriate carrier, for example, liposomes, or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7, 27 (1984)).

Pharmaceutical compositions suitable for injectable use include sterile aqueous solutions (where water soluble) or dispersions and sterile powders for the extemporaneous preparation of sterile injectable solutions or dispersion. In all cases, the composition must be sterile and must be fluid to the extent that easy syringability exists. It must be stable under the conditions of manufacture and storage and must be preserved against the contaminating action of microorganisms such as bacteria and fungi.

The vehicle can be a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, isotonic agents are included, for example, sugars, sodium chloride, or polyalcohols such as mannitol and sorbitol, in the composition. Prolonged absorption of the injectable compositions can be brought about by including an agent that delays absorption, for example, aluminum monostearate or gelatin, in the compositions.

Sterile injectable solutions can be prepared by incorporating the therapeutic agents in the required amount in an appropriate solvent with one or a combination of ingredients enumerated above, as required, followed by filtered sterilization. Generally, dispersions are prepared by incorporating the therapeutic agents into a sterile vehicle which contains a basic dispersion medium and the required other ingredients from those enumerated above. In the case of sterile powders for the preparation of sterile injectable solutions, the methods of preparation are vacuum drying and freeze-drying which yields a powder of the active ingredient (i.e., the therapeutic agent) plus any additional desired ingredient from a previously sterile-filtered solution thereof.

The therapeutic agents can be orally administered, for example, with an inert diluent or an assimilable edible carrier. The therapeutic agents and other ingredients may also be enclosed in a hard or soft shell gelatin capsule, compressed into tablets, or incorporated directly into the subject's diet. For oral therapeutic administration, the therapeutic agents may be incorporated with excipients and used in the form of ingestible tablets, buccal tablets, troches, capsules, elixirs, suspensions, syrups, wafers, and the like. The percentage of any therapeutic agent in the compositions and preparations may, of course, be varied. The amount of the therapeutic agents in such therapeutically useful compositions is such that a suitable dosage will be obtained.

It is especially advantageous to formulate parenteral compositions in dosage unit form for ease of administration and uniformity of dosage. Dosage unit form as used herein refers to physically discrete units suited as unitary dosages for the subjects to be treated; each unit containing a predetermined quantity of therapeutic agent calculated to produce the desired therapeutic effect in association with the required pharmaceutical vehicle. The specification for the dosage unit forms of the invention are dictated by and directly dependent on (a) the unique characteristics of the therapeutic agent and the particular therapeutic effect to be achieved, and (b) the limitations inherent in the art of compounding such a therapeutic agent for the treatment of amyloid deposition in subjects.

The present invention therefore includes pharmaceutical formulations comprising the agents described herein, including pharmaceutically acceptable salts thereof, in pharmaceutically acceptable carriers for aerosol, oral, and parenteral administration. Also, the present invention includes such agents, or salts thereof, which have been lyophilized and which may be reconstituted to form pharmaceutically acceptable formulations for administration, as by intravenous, intramuscular, or subcutaneous injection. Administration may also be intradermal or transdermal.

In accordance with the present invention, an agent as described herein, and pharmaceutically acceptable salts thereof, may be administered orally or through inhalation as a solid, or may be administered intramuscularly or intravenously as a solution, suspension or emulsion. Alternatively, the agents or salts may also be administered by inhalation, intravenously, or intramuscularly as a liposomal suspension.

Pharmaceutical formulations are also provided which are suitable for administration as an aerosol, by inhalation. These formulations comprise a solution or suspension of the desired agent described herein, or a salt thereof, or a plurality of solid particles of the agent or salt. The desired formulation may be placed in a small chamber and nebulized. Nebulization may be accomplished by compressed air or by ultrasonic energy to form a plurality of liquid droplets or solid particles comprising the agents or salts. The liquid droplets or solid particles should have a particle size in the range of about 0.5 to about 5 microns. The solid particles can be obtained by processing an agent as described herein, or a salt thereof, in any appropriate manner known in the art, such as by micronization. The size of the solid particles or droplets will be, for example, from about 1 to about 2 microns. In this respect, commercial nebulizers are available to achieve this purpose.

A pharmaceutical formulation suitable for administration as an aerosol may be in the form of a liquid, the formulation will comprise a water-soluble agent as described herein, or a salt thereof, in a carrier that comprises water. A surfactant may be present, which lowers the surface tension of the formulation sufficiently to result in the formation of droplets within the desired size range when subjected to nebulization.

Peroral compositions also include liquid solutions, emulsions, suspensions, and the like. The pharmaceutically acceptable carriers suitable for preparation of such compositions are well known in the art. Typical components of carriers for syrups, elixirs, emulsions, and suspensions include ethanol, glycerol, propylene glycol, polyethylene glycol, liquid sucrose, sorbitol, and water. For a suspension, typical suspending agents include methyl cellulose, sodium carboxymethyl cellulose, tragacanth, and sodium alginate; typical wetting agents include lecithin and polysorbate 80; and typical preservatives include methyl paraben and sodium benzoate. Peroral liquid compositions may also contain one or more components such as sweeteners, flavoring agents and colorants disclosed above.

Pharmaceutical compositions may also be coated by conventional methods, typically with pH or time-dependent coatings, such that the subject agent is released in the gastrointestinal tract in the vicinity of the desired topical application, or at various times to extend the desired action. Such dosage forms typically include, but are not limited to, one or more of cellulose acetate phthalate, polyvinylacetate phthalate, hydroxypropyl methyl cellulose phthalate, ethyl cellulose, waxes, and shellac.

Other compositions useful for attaining systemic delivery of the subject agents include sublingual, buccal, and nasal dosage forms. Such compositions typically comprise one or more of soluble filler substances such as sucrose, sorbitol and mannitol; and binders such as acacia, microcrystalline cellulose, carboxymethyl cellulose and hydroxypropyl methyl cellulose. Glidants, lubricants, sweeteners, colorants, antioxidants, and flavoring agents disclosed above may also be included.

The compositions of this invention can also be administered topically to a subject, e.g., by the direct laying on or spreading of the composition on the epidermal or epithelial tissue of the subject, or transdermally via a “patch.” Such compositions include for example, lotions, creams, solutions, gels, and solids. These topical compositions may comprise an effective amount, usually at least about 0.1%, or from about 1% to about 5%, of an agent of the invention. Suitable carriers for topical administration typically remain in place on the skin as a continuous film, and resist being removed by perspiration or immersion in water. Generally, the carrier is organic in nature and capable of having dispersed or dissolved therein the therapeutic agent. The carrier may include pharmaceutically acceptable emolients, emulsifiers, thickening agents, solvents and the like.

Active agents are administered at a therapeutically effective dosage sufficient to inhibit amyloid deposition in a subject A “therapeutically effective” dosage inhibits amyloid deposition by, for example, at least about 10%, or by at least about 40%, or even by at least about 60%, or by at least about 80% relative to untreated subjects. In the case of an Alzheimer's subject, a “therapeutically effective” dosage stabilizes cognitive function or prevents a further decrease in cognitive function (i.e., preventing, slowing, or stopping disease progression). The present invention accordingly provides therapeutic drugs. By “therapeutic” or “drug” is meant an agent having a beneficial ameliorative or prophylactic effect on a specific disease or condition in a living human or non-human animal.

A therapeutically effective dosage may decrease the levels of amyloid proteins, e g., Aβ40 and/or Aβ 42, in the CSF or plasma of a subject by, for example, at least about 15%, or by at least about 20%, or by at least about 25%, or by at least about 40%, or even by at least about 60%, or by at least about 80% relative to untreated subjects. Alternatively, a therapeutically effective dosage may increase the levels of amyloid proteins, e.g., Aβ-40 and/or Aβ42, in the plasma of a subject by, for example, at least about 15%, or by at least. about 20%, or by at least about 25%, or by at least about 40%, or even by at least about 60%, or by at least about 80% relative to untreated subjects. In another embodiment, a therapeutically effective dosage may improve the ADAS-Cog score of a subject by, for example, at least about one point, or by at least about 2 points, or by at least about three points, or by at least about 5 points, or by at least about 10 points, or by at least about 20 points.

Toxicity and therapeutic efficacy of such agents can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and can be expressed as the ratio LD50/ED50, and usually a larger therapeutic index is more efficacious. While agents that exhibit toxic side effects may be used, care should be taken to design a delivery system that targets such agents to the site of affected tissue in order to minimize potential damage to uninfected cells and, thereby, reduce side effects.

It is understood that appropriate doses depend upon a number of factors within the ken of the ordinarily skilled physician, veterinarian, or researcher. The dose(s) of the small molecule will vary, for example, depending upon the identity, size, and condition of the subject or sample being treated, further depending upon the route by which the composition is to be administered, if applicable, and the effect which the practitioner desires the small molecule to have upon the nucleic acid or polypeptide of the invention. Exemplary doses include milligram or microgram amounts of the small molecule per kilogram of subject or sample weight (e.g., about 1 microgram per kilogram to about 500 milligrams per kilogram, about 100 micrograms per kilogram to about 5 milligrams per kilogram, or about 1 microgram per kilogram to about 50 micrograms per kilogram). It is furthermore understood that appropriate doses depend upon the potency with respect to the expression or activity to be modulated. Such appropriate doses may be determined using the assays described herein. When one or more of these small molecules is to be administered to an animal (e.g., a human) in order to modulate expression or activity of a polypeptide or nucleic acid of the invention, a physician, veterinarian, or researcher may, for example, prescribe a relatively low dose at first, subsequently increasing the dose until an appropriate response is obtained. In addition, it is understood that the specific dose level for any particular animal subject will depend upon a variety of factors including the activity of the specific agent employed, the age, body weight, general health, gender, and diet of the subject, the time of administration, the route of administration, the rate of excretion, any drug combination, and the degree of expression or activity to be modulated.

The ability of an agent to inhibit amyloid deposition can be evaluated in an animal model system that maybe predictive of efficacy in inhibiting amyloid deposition in human diseases, such as a transgenic mouse expressing human APP or other relevant animal models where Aβ deposition is seen. Likewise, the ability of an agent to prevent or reduce cognitive impairment in a model system may be indicative of efficacy in humans. Alternatively, the ability of a agent can be evaluated by examining the ability of the agent to inhibit amyloid fibril formation in vitro, e.g., using a fibrillogenesis assay such as that described herein, including a ThT, CD, or EM assay. Also the binding of an agent to amyloid fibrils may be measured using a MS assay as described herein.

Pharmaceutically Acceptable Salts

Certain embodiments of the present agents can contain a basic functional group, such as amino or alkylamino, and are, thus, capable of forming pharmaceutically acceptable salts with pharmaceutically acceptable acids. The term “pharmaceutically acceptable salts” in this respect, refers to the relatively non-toxic, inorganic and organic acid addition salts of agents of the present invention. These salts can be prepared in situ during the final isolation and purification of the agents of the invention, or by separately reacting a purified agent of the invention in its free base form with a suitable organic or inorganic acid, and isolating the salt thus formed.

Representative salts include the hydrohalide (including hydrobromide and hydrochloride), sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, mesylate, glucoheptonate, lactobionate, 2-hydroxyethylsulfonate, and laurylsulphonate salts and the like. See, e.g., Berge et al., J. Pharm. Sci. 66, 1-19 (1977).

In other cases, the agents of the present invention may contain one or more acidic functional groups and, thus, are capable of forming pharmaceutically acceptable salts with pharmaceutically acceptable bases. The term “pharmaceutically acceptable salts” in these instances refers to the relatively non-toxic, inorganic and organic base addition salts of agents of the present invention.

These salts can likewise be prepared in situ during the final isolation and purification of the agents, or by separately reacting the purified agent in its free acid form with a suitable base, such as the hydroxide, carbonate or bicarbonate of a pharmaceutically acceptable metal cation, with ammonia, or with a pharmaceutically acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine, and the like.

“Pharmaceutically acceptable salts” also includes, for example, derivatives of agents modified by making acid or base salts thereof, as described further below and elsewhere in the present application. Examples of pharmaceutically acceptable salts include mineral or organic acid salts of basic residues such as amines; and alkali or organic salts of acidic residues such as carboxylic acids. Pharmaceutically acceptable salts include the conventional non-toxic salts or the quaternary ammonium salts of the parent agent formed, for example, from non-toxic inorganic or organic acids. Such conventional non-toxic salts include those derived from inorganic acids such as hydrochloric, hydrobromic, sulfuric, sulfamic, phosphoric, and nitric acid; and the salts prepared from organic acids such as acetic, propionic, succinic, glycolic, stearic, lactic, malic, tartaric, citric, ascorbic, palmoic, maleic, hydroxymaleic, phenylacetic, glutamic, benzoic, salicylic,'sulfanilic, 2-acetoxybenzoic, fumaric, toluenesulfonic, methanesulfonic, ethane disulfonic, oxalic, and isethionic acid. Pharmaceutically acceptable salts may be synthesized from the parent agent that contains a basic or acidic moiety by conventional chemical methods. Generally, such salts may be prepared by reacting the free acid or base forms of these agents with a stoichiometric amount of the appropriate base or acid in water or in an organic solvent, or in a mixture of the two.

Alkylsulfonic Acids

As is discussed above, the invention relates to methods for treating or preventing an amyloid-β related disease by administering at least two agents, each of which exerts a therapeutic effect and is useful in treating or preventing a neurological disease. As is discussed above, the first agent of the invention is selected from alkylsulfonic acids that are useful for treating or preventing an amyloid-β related disease. The second agent is a peptide, a peptidomimetic compound, or an immune system modulator (eg., an antibody), as discussed above. Also as is discussed above, one or more agents in addition to the first and second agents can be used in the invention. Details and examples of sulfonic acid-based drugs that can be used in the invention are provided here, while information concerning other agents that are used in the invention is presented further below.

Compositions of alkylsulfonic acids, including, for example, 3-amino-1-propanesulfonic acid and certain salts thereof, have been shown to be useful in the treatment of amyloid-β related diseases, including Alzheimer's disease and cerebral amyloid angiopathy (see, e.g., WO 96128187, WO 01/85093, and U.S. Pat. No. 5,840,294). The anionic group of the composition is believed to inhibit an interaction between an amyloidogenic protein and a glycosaminoglycan (GAG) or proteoglycan constituent of a basement membrane to thus inhibit amyloid deposition.

The term “alkylsulfonic acid” includes substituted or unsubstituted alkylsulfonic acids, and substituted or unsubstituted lower alkylsulfonic acids. Amino-substituted compounds are especially noteworthy and the invention pertains to substituted- or unsubstituted-amino-substituted alkylsulfonic acids, and substituted- or unsubstituted-amino-substituted lower alkylsulfonic acids, an example of which is 3-amino-1-propanesulfonic acid.

The methods and pharmaceutical compositions of the invention are therefore related to a first agent that is a substituted or unsubstituted alkylsulfonic acid, substituted or unsubstituted alkylsulfuric acid, substituted or unsubstituted alkylthiosulfonic acid, substituted or unsubstituted alkylthiosulfuric acid, or an ester or amide thereof, including pharmaceutically acceptable salts thereof. For example, the invention relates to a first agent that is a substituted or unsubstituted alkylsulfonic acid, or an ester or amide thereof, including pharmaceutically acceptable salts thereof. In another embodiment, the invention pertains to a first agent that is a substituted or unsubstituted lower alkylsulfonic acid, or an ester or amide thereof, including pharmaceutically acceptable salts thereof. Similarly, the invention includes a first agent that is a (substituted- or unsubstituted-amino)-substituted alkylsulfonic acid, or an ester or amide thereof, including pharmaceutically acceptable salts thereof. In yet another embodiment, the first agent is a (substituted- or unsubstituted-amino)-substituted lower alkylsulfonic acid, or an ester or amide thereof, including pharmaceutically acceptable salts thereof.

As used herein, “alkyl” groups include saturated hydrocarbons having one or more carbon atoms, including straight-chain alkyl groups (e.g., methyl, ethyl, propyl, butyl, pentyl, hexyl, heptyl, octyl, nonyl, decyl, etc.), cyclic alkyl groups (or “cycloalkyl” or “alicyclic” or “carbocyclic” groups) (e.g., cyclopropyl; cyclopentyl, cyclohexyl, cycloheptyl, cyclooctyl, etc.), branched-chain alkyl groups (isopropyl, tent-butyl, sec-butyl, isobutyl, etc), and alkyl-substituted alkyl groups (e.g., alkyl-substituted cycloalkyl groups and cycloalkyl-substituted alkyl groups).

Accordingly, the invention relates to methods employing and compositions including substituted or unsubstituted alkylsulfonic acids that are substituted or unsubstituted straight-chain alkylsulfonic acids, substituted or unsubstituted cycloalkylsulfonic acids, and substituted or unsubstituted branched-chain alkylsulfonic acids. Also, it is noted that the term “alkylsulfonic acid” as used herein is to be interpreted as being synonymous with the term “alkanesulfonic acid.”

The structures of some of the compounds of the invention include stereogenic carbon atoms. It is to be understood that isomers arising from such asymmetry (e.g. all enantiomers and diastereomers) are included within the scope of this invention unless indicated otherwise. That is, unless otherwise stipulated, any chiral carbon center may be of either (R)- or (S)-stereochemistry. Such isomers can be obtained in substantially pure form by classical separation techniques and by stereochemically-controlled synthesis, In addition, the compounds of the present invention may exist in unsolvated as well as solvated forms with acceptable solvents such as water, THF, ethanol, and the like. In general, the solvated forms are considered equivalent to the unsolvated forms for the purposes of the present invention. The term “solvate” represents an aggregate that comprises one or more molecules of a compound, with one or more molecules of a pharmaceutical solvent, such as water, ethanol, and the like.

In certain embodiments, a straight-chain or branched-chain alkyl group may have 30 or fewer carbon atoms in its backbone, e.g., C₁-C₃₀ for straight-chain or C₃-C₃₀ for branched-chain. In certain embodiments, a straight-chain or branched-chain alkyl group may have 20 or fewer carbon atoms in its backbone, e.g., C₁-C₂₀ for straight-chain or C₃-C₂₀ for branched-chain, and more, for example, 18 or fewer. Likewise, example cycloalkyl groups have from 4-10 carbon atoms in-their ring structure, or 47 carbon atoms in the ring structure.

The term “lower alkyl” refers to alkyl groups having from 1 to 6 carbons in the chain, and to cycloalkyl groups having from 3 to 6 carbons in the ring structure. Unless the number of carbons is otherwise specified, “lower” as in “lower alkyl,” means that the moiety has at least one and less than about 8 carbon atoms. In certain embodiments, a straight-chain or branched-chain lower alkyl group has 6 or fewer carbon atoms in its backbone (e.g., C₁-C₆ for straight-chain, C₃-C₆ for branched-chain), for example, methyl, ethyl, propyl, isopropyl, butyl, isobutyl, sec-butyl, and tert-butyl. Likewise, cycloalkyl groups may have from 3-8 carbon atoms in their ring structure, for example, 5 or 6 carbons in the ring structure. The term “C₁-C₆” as in “C₁-C₆ alkyl” means alkyl groups containing 1 to 6 carbon atoms.

Moreover, unless otherwise specified the term alkyl includes both “unsubstituted alkyls” and “substituted alkyls,” the latter of which refers to alkyl groups having substituents replacing one or more hydrogens on one or more carbons of the hydrocarbon backbone. Such substituents may include, for example, alkenyl, alkynyl, halogeno, hydroxyl, alkylcarbonyloxy, arylcarbonyloxy, alkoxycarbonyloxy, aryloxycarbonyloxy, carboxylate, alkylcarbonyl, arylcarbonyl, alkoxycarbonyl, aminocarbonyl, alkylaminocarbonyl, dialkylaminocarbonyl, alkylthiocarbonyl, alkoxyl, phosphate, phosphonato, phosphinato, cyano, amino (including alkyl amino, dialkylamino, arylamino, diarylamino, and alkylarylamino), acylamino (including alkylcarbonylamino, arylcarbonylamino, carbamoyl and ureido), amidino, imino, sulfhydryl, alkylthio, arylthio, thiocarboxylate, sulfates, alkylsulfinyl, sulfonato, sulfamoyl, sulfonamido, nitro, trifluoromethyl, cyano, azido, heterocyclyl, alkylaryl, or aromatic (including heteroaromatic) groups.

The term “amine” or “amino,” as used herein, refers to an unsubstituted or substituted moiety of the formula —NR^{a}R^{b}, in which R^{a} and R^{b} are each independently hydrogen, alkyl, aryl, or heterocyclyl, or R^{a} and R^{b}, taken together with the nitrogen atom to which they are attached, form a cyclic moiety having from 3 to 8 atoms in the ring. Thus, the term amino includes cyclic amino moieties such as piperidinyl or pyrrolidinyl groups, unless otherwise stated. Thus, the term “alkylamino” as used herein means an alkyl group having an amino group attached thereto. Suitable alkylamino groups include groups having 1 to about 12 carbon atoms, for example, 1 to about 6 carbon atoms. The term amino includes compounds or moieties in which a nitrogen atom is covalently bonded to at least one carbon or heteroatom. The term “dialkylamino” includes groups wherein the nitrogen atom is bound to at least two alkyl groups. The term “arylamino” and “diarylamino” include groups wherein the nitrogen is bound to at least one or two aryl groups, respectively. The term “alkylarylamino” refers to an amino group that is bound to at least one alkyl group and at least one aryl group. The term “alkaminoalkyl” refers to an alkyl, alkenyl, or alkynyl group substituted with an alkylamino group. The term “amide” or “aminocarbonyl” includes compounds or moieties that contain a nitrogen atom that is bound to the carbon of a carbonyl or a thiocarbonyl group.

A “sulfonic acid” or “sulfonate” group is a —SO₃H or —SO₃⁻X⁺ group bonded to a carbon atom, where X⁺ is a cationic counter ion group. Similarly, a “sulfonic acid” compound has a —SO₃H or —SO₃⁻X⁺ group bonded to a carbon atom, where X⁺ is a cationic group. A “sulfate” as used herein is a —OSO₃H or —OSO₃⁻X⁺ group bonded to a carbon atom, and a “sulfuric acid” compound has a —SO₃H or —OSO₃⁻X⁺ group bonded to a carbon atom, where X⁺ is a cationic group. According to the invention, a suitable cationic group may be a hydrogen atom. In certain cases, the cationic group may actually be another group on the therapeutic compound that is positively charged at physiological pH, for example and amino group. A “counter ion” is required to maintain electroneutrality, and is pharmaceutically acceptable in the compositions of the invention. Compounds containing a cationic group covalently bonded to an anionic group may be referred to as an “internal salt.”

Unless otherwise specified, the chemical moieties of the compounds of the invention, including those groups discussed above, may be “substituted or unsubstituted.” In some embodiments, the term “substituted” is used to mean that the moiety has substituents placed on the moiety other than hydrogen (i.e., in most cases, replacing a hydrogen), which allow the molecule to perform its intended function. Examples of substituents include moieties selected from straight or branched alkyl (e.g., C₁-C₅), cycloalkyl (eg., C₃-C₈), amino groups (including —NH₂), —SO₃H, —OSO₃H, —CN, —NO₂, halogen (e.g. —F, —Cl, —Br, or —I), —CH₂OCH₃, —OCH₃, —SH, —SCH₃, —OH, and —CO₂H.

It will be understood that “substitution” or “substituted with” includes the implicit proviso that such substitution is in accordance with permitted valence of the substituted atom and the substituent, and that the substitution results in a stable compound, e.g. which does not spontaneously undergo transformation such as by rearrangement, cyclization, elimination, etc. As used herein, the term “substituted” is meant to include all permissible substituents of organic compounds. In a broad aspect, the permissible substituents include acyclic and cyclic, branched and unbranched, carbocyclic and heterocyclic, aromatic and nonaromatic substituents of organic compounds. The permissible substituents can be one or more and the same or different for appropriate organic compounds.

In some embodiments, a “substituent” may be, selected from the group consisting of, for example, halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl (including heteroaryl) groups.

One group of example alkylsulfonic acids have the following structure 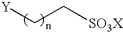
where Y is either an amino group (having the formula —NR^{a}R^{b}) or a sulfonic acid group (having the formula —SO₃⁻X⁺), n is an integer from 1 to 5, and X is hydrogen or a cationic group (e.g., sodium).

Some exemplary alkylsulfonic acids include the following:

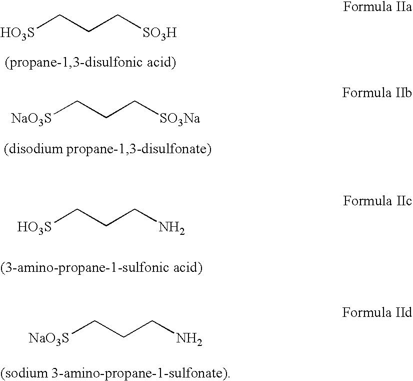

One embodiment of the invention is the use of 3-amino-1-propanesulfonic acid and pharmaceutically acceptable salts thereof as a first agent of the pharmaceutical compositions described herein and the methods of using them.

An “agent,” as in a “first agent” or a “second agent” is generally intended to describe a chemical compound of suitable purity for use in a pharmaceutical preparation. In some cases, the agent is a “small molecule,” that is, a compound that that is not itself the product of gene transcription or translation (e.g., protein, RNA, or DNA) and has a low molecular weight, e.g., less than about 2500. In other cases, the agent may be a biological product, such as an antibody or an immunogenic peptide.

Alkylsulfonic acids may be prepared by the methods illustrated in the general reaction schemes as, for example, described in U.S. Pat. Nos, 5,643,562; 5,972,328; 5,728,375; 5,840,294; 4,657,704; and U.S. Provisional Patent Application No. 60/482,058, filed Jun. 23, 2003, entitled “Synthetic Process for Preparing Compounds for Treating Amyloidosis,” or by modifications thereof, using readily available starting materials, reagents and conventional synthesis procedures. In these reactions, it is also possible to make use of variants that are in themselves known, but are not mentioned. Functional and structural equivalents of the agents described herein and which have the same general properties, wherein one or more simple variations of substituents are made which do not adversely affect the essential nature or the utility of the agent may be prepared according to a variety of methods known in the art, and can be used.

In general, the agents used in the present invention may be prepared by the methods illustrated in the general reaction schemes as, for example, described below, or by modifications thereof, using readily available starting materials, reagents and conventional synthesis procedures. In these reactions, it is also possible to make use of variants that are in themselves known, but are not mentioned here. Functional and structural equivalents of the agents described herein and which have the same general properties, wherein one or more simple variations of substituents are made which do not adversely affect the essential nature or the utility of the agent. The agents of the present invention may be readily prepared in accordance with the synthesis schemes and protocols described herein, as illustrated in the specific procedures provided. However, those skilled in the art will recognize that other synthetic pathways for forming the agents of this invention may be used, and that the following is provided merely by way of example, and is not limiting to the present invention. See, e.g., “Comprehensive Organic Transformations,” 2^{nd} Ed., by R. C. Larock, John Wiley & Sons, Ltd. (1999); “March's Advanced Organic Chemistry,” 5^{th} Ed., by M. B. Smith and J. March, John Wiley & Sons, Ltd. (2000), and “Reagents for Organic Synthesis,” Vol. I-XX, by M. Fieser and L. Fieser, John Wiley & Sons (2000). It will be further recognized that various protecting and deprotecting strategies will be employed that are standard in the art (see, e.g., “Protective Groups in Organic Synthesis,” 3^{rd} Ed., by Greene, John Wiley & Sons, Ltd. (1999)). Those skilled in the relevant arts will recognize that the selection of any particular protecting group (e.g., amine and carboxyl protecting groups) will depend on the stability of the protected moiety with regards to the subsequent reaction conditions and will understand the appropriate selections. Further illustrating the knowledge of those skilled in the art is the following sampling of the extensive chemical literature: “Comprehensive Asymmetric Catalysis,” by Jacobsen et al., Springer Verlag (1999) “Chemistry of the Amino Acids” by Greenstein et al, John Wiley & Sons, Inc., New York (1961); Ocain et at, J. Med. Chem. 31, 2193-2199 (1988); Gordon et al, J. Med. Chem. 31, 2199-2110 (1988); “Practice of Peptide Synthesis” by Bodansky et at, Springer-Verlag, New York (1984); “Asymmetric Synthesis: Construction of Chiral Molecules Using Amino Acids” by Coppola et al., John Wiley & Sons, Inc., New York (1987); “The Chemical Synthesis of Peptides” by Jones, Oxford University Press, New York (1991); and “Introduction of Peptide Chemistry” by Bailey, John Wiley & Sons, Inc., New York (1992). [The chemical structures herein are drawn according to the conventional standards known in the art. Thus, where an atom, such as a carbon atom, as drawn appears to have an unsatisfied valency, then that valency is assumed to be satisfied by a hydrogen atom even though that hydrogen atom is not necessarily explicitly drawn. The structures of some of the compounds of this invention include stereogenic carbon atoms. It is to be understood that isomers arising from such asymmetry (e.g., all enantiomers and diastereomers) are included within the scope of this invention unless indicated otherwise. That is, unless otherwise stipulated, any chiral carbon center may be of either (R)- or (S)-stereochemistry. Such isomers can be obtained in. substantially pure form by classical separation techniques and by stereochemically-controlled synthesis. Furthermore, alkenes can include either the E- or Z-geometry, where appropriate. In addition, the compounds of the present invention may exist in unsolvated as well as solvated forms with acceptable solvents such as water, THF, ethanol, and the like. In general, the solvated forms are considered equivalent to the unsolvated forms for the purposes of the present invention.

In an embodiment, the invention pertains, at least in part to a pharmaceutical composition having a first agent that is a compound of Formula I-A: 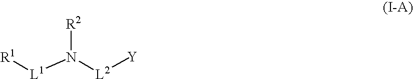
wherein:

R¹ is a substituted or unsubstituted cycloalkyl, aryl, arylcycloalkyl, bicyclic or tricyclic ring, a bicyclic or tricyclic fused ring group, or a substituted, or unsubstituted C₂-C₁₀ alkyl group;

R² is selected from a group consisting hydrogen, alkyl, mercaptoalkyl, alkenyl, alkynyl, cycloalkyl, aryl,arylalkyl, thiazolyl, triazolyl, imidazolyl, benzothiazolyl, arid benzoimidazolyl;

Y is SO₃⁻X⁺, OSO₃⁻X⁺, or SSO₃⁻X⁺;

X+ is hydrogen, a cationic group, or an ester-forming group (i.e., as in a prodrug, which are described elsewhere herein); and

each of L¹ and L² is independently a substituted or unsubstituted C -C₅ alkyl group or absent, or a pharmaceutically acceptable salt thereof, provided that when R¹ is alkyl, L¹ is absent.

In another embodiment, the invention pertains, at least in part a pharmaceutical composition having a first agent that is a compound of Formula II-A: 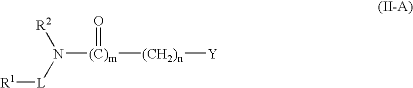
wherein:

R¹ is a substituted or unsubstituted cyclic, bicyclic, tricyclic, or benzoheterocyclic group or a substituted or unsubstituted C₂-C₁₀ alkyl group;

R² is hydrogen, alkyl, mercaptoalkyl, alkenyl, alkynyl, cycloalkyl, aryl, arylalkyl, thiazolyl, triazolyl, imidazolyl, benzothiazolyl, benzoimidazolyl, or linked to R¹ to form a heterocycle;

Y is SO₃⁻X⁺, OSO₃⁻X⁺, or SSO₃⁻X⁺;

X⁺ is hydrogen, a cationic group, or an ester forming moiety;

m is 0 or 1;

n is 1, 2, 3, or 4;

L is substituted or unsubstituted C₁-C₃ alkyl group or absent, or a pharmaceutically acceptable salt thereof, provided that when R¹ is alkyl, L is absent.

In yet another embodiment, the invention pertains, at least in part to a pharmaceutical composition having a first agent that is a compound of Formula III-A: 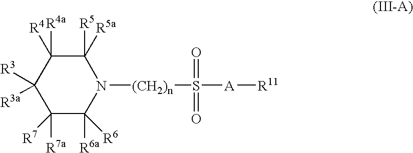
wherein:

A is nitrogen or oxygen;

R¹¹ is hydrogen, salt-forming cation, ester forming group, —(CH₂)ₓ-Q or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

x is 0, 1, 2, 3, or 4;

n is 0, 1, 2, 3, 4, 5, 6, 7, 8, 9, or 10;

R³, R^{3a}, R⁴, R^{4a}, R⁵, R^{5a}, R⁶, R^{6a}, R⁷, and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, amidino, tetrazolyl, or two R groups on adjacent ring atoms taken together with the ring atoms form a double bond, provided that one of R³, R^{3a}, R⁴, R^{4a}, R⁵, R^{5a}, R⁶, R^{6a}, R⁷, and R^{7a} is a moiety of Formula IIIa-A: 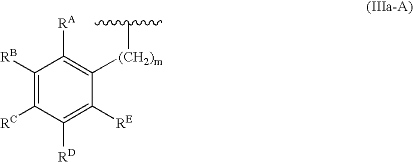
wherein:

m is 0, 2, 3, or 4;

R⁸, R⁹, R¹⁰, R¹¹, and R¹² are independently selected from a group of hydrogen, halogen, hydroxyl, alkyl, alkoxyl, halogenated alkyl, mercaptoalkyl, alkenyl, alkynyl, cycloalkyl, aryl, cyano, amidino, thiazolyl, triazolyl, imidazolyl, tetrazolyl, benzothiazolyl, and benzoimidazoly; and pharmaceutically acceptable salts and esters thereof, provided that said compound is not 3-(4-phenyl-1,2,3,6tetrahydro-1-pyridyl)-1-propanesulfonic acid.

In yet another embodiment, the invention pertains at least in part to a pharmaceutical composition having a first agent that is a compound of Formula IV: 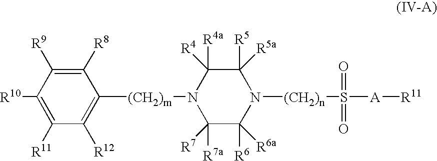
wherein:

A is nitrogen or oxygen;

R¹¹ is hydrogen, salt-forming cation, ester forming group, —(CH₂)ₓ-Q or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

x is 0, 1, 2, 3, or4;

n is 0, 1, 2, 3, 4, 5, 6, 7, 8, 9, or 10;

R⁴, R^{4a}, R⁵, R^{5a}, R⁶, R^{6a}, R⁷, and R^{7a} are each independently hydrogen, alkyl, mercaptoalkyl, alkenyl, alkynyl, cycloalkyl, aryl, alkylcarbonyl, arylcarbonyl, alkoxycarbonyl, cyano, halogen, amino, amidino, tetrazolyl, R⁴ and R⁵ are taken together, with the ring atoms they are attached to, form a double bond, or R⁶ and R⁷ are taken together, with the ring atoms they are attached to, form a double bond;

m is 0, 1, 2, 3, or 4;

R⁸, R⁹, R¹⁰, R¹¹, and R¹² are independently selected from a group of hydrogen, halogen, hydroxyl, alkyl, alkoxyl, halogenated-alkyl, mercaptoalkyl, alkenyl, alkynyl, cycloalkyl, aryl, cyano, amidino, thiazolyl, triazolyl, imidazolyl, tetrazolyl, benzothiazolyl, and benzoimidazolyl, and pharmaceutically acceptable salts and esters thereof.

In another embodiment, the invention includes a pharmaceutical composition having a first agent that is a compound of Formula V-A: 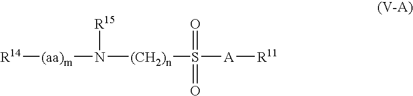
wherein:

A is nitrogen or oxygen;

R¹¹ is hydrogen, salt-forming cation, ester forming group, —(CH₂)ₓ-Q or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

x is 0, 1, 2, 3, or 4;

n is 0, 1, 2, 3, 4, 5, 6, 7, 8, 9, or 10;

aa is a natural or unnatural amino acid residue;

m is 0, 1, 2, or 3;

R¹⁴ is hydrogen or protecting group;

R¹⁵ is hydrogen, alkyl or aryl, and pharmaceutically acceptable salts and prodrugs thereof.

In another embodiment, the invention includes a pharmaceutical composition having a first agent that is a compound of the Formula VI-A: 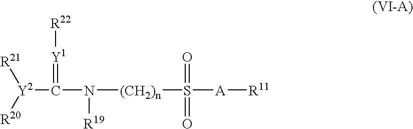
wherein:

n is 1, 2, 3, 4, 5, 6, 7, 8, 9, or 10;

A is oxygen or nitrogen;

R¹¹ is hydrogen, salt-forming cation, ester forming group, —(CH₂)ₓ-Q, or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

x is 0, 1, 2, 3, or 4;

R¹⁹ is hydrogen, alkyl or aryl;

Y¹ is oxygen, sulfur, or nitrogen;

Y² is carbon, nitrogen, or oxygen;

R²⁰ is hydrogen, alkyl, amino, mercaptoalkyl, alkenyl, alkynyl, cycloalkyl, aryl, arylalkyl, thiazolyl, triazolyl, tetrazolyl, imidazolyl, benzothiazolyl, or benzoimidazolyl;

R²¹ is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl, or absent if Y² is oxygen;

R²² is hydrogen, alkyl, mercaptoalkyl, alkenyl, alkynyl, cycloalkyl, aryl, arylalkyl, thiazolyl, triazolyl, tetrazolyl, imidazolyl, benzothiazolyl, benzoimidazolyl; or R²² is hydrogen, hydroxyl, alkoxy or aryloxy if Y¹ is nitrogen; or R²² is absent if Y¹ is oxygen or sulfur; or R²² and R²¹ may be linked to form a cyclic moiety if Y¹ is nitrogen;

or pharmaceutically acceptable salts thereof.

In another embodiment, the invention includes a pharmaceutical composition having a first agent that is a compound of Formula VII-A: 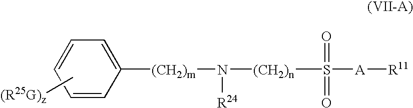
wherein:

n is 2, 3, or 4;

A is oxygen or nitrogen;

R¹¹ is hydrogen, salt-forming cation, ester forming group, —(CH₂)ₓ-Q, or when A is nitrogen, A and R¹¹ taken together may be the residue of a natural or unnatural amino acid or a salt or ester thereof;

Q is hydrogen, thiazolyl, triazolyl, imidazolyl, benzothiazolyl, or benzoimidazolyl;

x is 0, 1, 2, 3, or 4;

G is a direct bond or oxygen, nitrogen, or sulfur;

z is 0, 1, 2, 3, 4, or 5;

m is 0 or 1;

R²⁴ is selected from a group consisting hydrogen, alkyl, mercaptoalkyl, alkenyl, alkynyl, aroyl, alkylcarbonyl, aminoalkylcarbonyl, cycloalkyl, aryl, arylalkyl, thiazolyl, triazolyl, imidazolyl, benzothiazolyl, and benzoimidazolyl;

each R²⁵ is independently selected from hydrogen, halogen, cyano, amidino, hydroxyl, alkoxy, thiol, amino, nitro, alkyl, aryl, carbocyclic, or heterocyclic, and pharmaceutically acceptable salts thereof.

The ability of a sulfonic acid compound used in the invention to inhibit an interaction between an amyloidogenic protein and a glycoprotein or proteoglycan constituent of a basement membrane can be assessed by an in vitro binding, assay, such as that described herein or in U.S. Pat. No. 5,164,295. Briefly, a solid support such as a polystyrene microtiter plate is coated with an amyloidogenic protein (eg., serum amyloid A protein or β-amyloid precursor protein (β-APP)) and any residual hydrophobic surfaces are blocked. The coated solid support is incubated with various concentrations of a constituent of basement membrane, for example HSPG, either in the presence or absence of a compound to be tested. The solid support is washed extensively to remove unbound material. The binding of the basement membrane constituent (e.g., HSPG) to the amyloidogenic protein (e.g., β-APP) is then measured using an antibody directed against the basement membrane constituent that is conjugated to a detectable substance (e.g., an enzyme, such as alkaline phosphatase) by detecting the detectable substance. A compound which inhibits an interaction between an amyloidogenic protein and a glycoprotein or proteoglycan constituent of a basement membrane will reduce the amount of substance detected (e.g., will inhibit the amount of enzyme activity detected). In one embodiment, a therapeutic compound of the invention may interact with a binding site for a basement membrane glycoprotein or proteoglycan in an amyloidogenic protein and thereby inhibit the binding of the amyloidogenic protein to the basement membrane constituent. Basement membrane glycoproteins and proteoglycans include laminin, collagen type IV, fibronectin and heparan sulfate proteoglycan (HSPG), perlecan, and agrin. In a similar embodiment, the therapeutic compound inhibits an interaction between an amyloidogenic protein and HSPG. Consensus binding site motifs for HSPG in amyloidogenic proteins have been described (see, e.g., Cardin et al., Arteriosclerois. 9, 21-32 (1989)). Characterization of sulfonic acid compounds for use in the present invention can also be carried out using other assays, as determined to be appropriate by those of skill in this art For example, the ability of a sulfonic acid compound to bind to an amyloid protein can be assessed by ES/MS. Compounds are screened for their abilities to interact with soluble amyloid protein prior its structural change that leads to fibril formation. Binding to the amyloid protein may maintain the protein in a random coil structure and favor its clearance prior adapting a β-sheet structure. A compound with the ability to maintain an amyloid protein in a random coil/soluble structure can be assessed in vivo by measuring the level of amyloid in the blood stream, in a specific organ, or in the CSF.

Vaccine and Peptide Approaches

As is noted above, the alkylsulfonic acid compounds described herein can be used in the methods and compositions of the invention in combination with peptides, peptidomimetic, and immune system modulators (e.g., antibodies) to prevent and to treat Aβ-related diseases and conditions. Many studies have shown that the toxic properties of Aβ can be blocked using an immunotherapeutic approach. An immune response generated against specific forms of Aβ may neutralize its neurotoxic effects, inhibit its fibril formation, or favor its clearance before it triggers neuronal damage. Such vaccination can prevent the development of Alzheimer's disease in individuals at risk of developing the disease, or even stop its progression following early diagnosis.

The first insight into the beneficial effects of mounting a humoral immune response to Aβ stemmed from the work of Solomon and colleagues who used monoclonal antibodies directed toward the N-terminal region of Aβ that not only prevented soluble Aβ from forming fibrils, but also led to the disaggregation of amyloid fibrils (Solomon et al., Proc. Natl Acad. Sci. U.S.A. 94, 4109-4112 (1997)). In cell culture systems, these antibodies inhibited the neurotoxic effects of fibrillar Aβ (fAβ). These authors further found that antibodies recognizing an epitope between residues 3 to 6 of Aβ (EFRH) exhibited the best inhibitory activity profile at blocking Aβ aggregation and resolubilizing preformed aggregates (Frenkel et al., Proc. Natl. Acad. Sci. U.S.A. 97,11455-11459(2000).

Recently, active immunization with different forms of Aβ peptide has been shown to decrease brain Aβ deposition and improve cognitive performance in mouse models of Alzheimer's disease. Certain peripherally administered anti-Aβ antibodies have similar effects. (For review, see, e.g., Holtzman et al., Adv. Drug Deliv. Rev. 54, 1603-1613 (2002); and Gervais and Tremblay, “Immunotherapy in Alzheimer's Disease,” In Alzheimer 's Disease and Related Disorders Annual 2002, Gauthier and Cummings (eds.), Martin Dunitz, London, 53-66, 2002.) Studies have shown that mounting an immune response to Aβ leads to the production of antibodies capable of clearing senile plaques, preventing further deposition of Aβ, thus facilitating the degradation or clearance of soluble Aβ. Such antibodies can thereby protect the brain from the neurotoxic effects of Aβ and prevent the plaque-associated inflammatory response, which exacerbates the degenerative process. Numerous studies to test the immunotherapeutic approach have been performed using Alzheimer's disease transgenic mouse models. These studies are summarized in Table 1.

TABLE 1

| | | | | Change in Cerebral Levels | |
|---|---|---|---|---|---|
| Reference | Model | Treatment¹ | Route² | Aβ³ | Amyloid Burden |
| Bacskal 2001 | PDAPP | Monoclonal 10D5 | Topic | | −65% |
| | | Monoclonal 16B5 | Topic | | −20% |
| Bard 2000 | PDAPP | Monoclonal 10D5 | IP | 42: −65% | −90% |
| | | Monoclonal 21F12 | IP | 42: 0% | 0% |
| | | Polyclonal | IP | 42: −65% | −80% |
| | | Monoclonal 3D6 | IP | | −85% |
| | | Monoclonal 16C11 | IP | | 0% |
| Das 2001 | Tg2576 | fAβ₄₂ | IP | −70% | −85% |
| DeMattos 2001 | PDAPP | Monoclonal m266 | IP | Total: −70% | Decreased |
| Janus 2000 | TgCRND8 | fAβ₄₂ | IP | Total: 0% | −50% |
| Morgan 2000 | Tg2576 | fAβ₄₂ | SC | | −80% |
| | Tg2576 + PS1 | fAβ₄₂ | SC | | −20% |
| Shenk 1999 | PDAPP | fAβ₄₂ | IP | Total: −80% | −95% |
| Sigurdsson 2001 | Tg2576 | K6Aβ₁₋₃₀ | SC | Total: −55% | −85% |
| Vehmas 2001 | TgAPPswe | fAβ₄₂ | IP | 40: +100%; | −45% |
| | | | | 42: 0% | |
| Weiner 2000 | PDAPP | Aβ₄₀ | oral | Total: 0% | 0% |
| | | | nasal | Total: −50% | −60% |

¹All antibodies shown were raised against Aβ.
²Abbreviations: IP, Intraperitoneal; IV, intravenous; SC, subcutaneous; Topic, topic application on the neocortex.
³Total, total Aβ levels; 40, levels of Aβ₄₀; 42, levels of Aβ₄₂.

Results from active as well as passive immunization experiments in transgenic mice show that antibodies cross the blood-brain barrier (BBB), associate with existing plaques, and trigger their clearance through an antibody-mediated phagocytosis process by activated microglia The mechanism by which sufficient titers of anti-Aβ antibodies enter the brain to access the plaques is unknown at this time. It is generally accepted that antibodies generated in the periphery have limited access to the central nervous system (CNS) due to the low permeability of the BBB, with as little as 0.1% of the circulating levels being found within the brain (Hunter et al., J. Neurol. Sci. 150, 103-113 (1997)). Other mechanisms of action whereby antibodies would mainly exert their activity systemically have also been proposed (DeMattos et al., Proc. Natl Acad. Sci. U.S.A. 98, 8850-8855 (2001)). 56.

Schenk et al. showed that, in a transgenic mouse model of brain amyloidosis (as seen in Alzheimer's disease), immunization with Aβ peptide inhibits the formation of amyloid plaques and the associated dystrophic neurites (Schenk et al., Nature 400, 173-177 (1999)). In that study, a vaccine using the human aggregated all-L peptide as immunogen prevented the formation of β-amyloid plaque, astrogliosis, and neuritic dystrophy in vaccinated transgenic mice. More recently, studies have shown that antibodies against β-amyloid can slow cognitive decline in Alzheimer's disease (Hock et al., Neuron 38, 547-554 (2003)). Another immunization approach against β-amyloid is based on the use of truncated Aβ peptides lacking the hydrophobic carboxyl terminus (e.g., peptides corresponding to positions 1-30) that include one or two substitutions of hydrophobic amino acids with charged amino acids, such as lysine, aspartic acid, or glutamic acid (see U.S. Patent Application Publication No. 20020077288).

Using the monoclonal antibody m266 directed against the central core of Aβ, De Mattos and colleagues found that passive administration of m266 antibodies peripherally leads to a decrease of amyloid burden in PDAPP^{+/+} mice (DeMattos et al., Proc. Natl. Acad. Sci. U.S.A. 98, 8850-8855 (2001)). The antibody appears to act as a peripheral sink by binding Aβ in the periphery and dramatically increasing levels of circulating m266-bound Aβ. This report shows that sequestration of Aβ in the plasma favors further transport of Aβ from CNS into the periphery and leads to a reduction of CNS Aβ levels and amyloid deposits. The central core domain of Aβ recognized by m266 antibodies contains the region that undergoes conformational transition and leads to the formation of β-sheets. Its binding to antibodies could stabilize its conformation preventing formation of β-sheets in addition to shifting the CNS-plasma equilibrium toward the periphery. The 10D5 and 3D6 antibodies, which recognize the amino terminus of Aβ, were previously found to significantly decrease Aβ deposition in PDAPP mice. In contrast to m266, these antibodies were shown to enter the brain, bind to Aβ plaques, and trigger their clearance by activated microglia. Antibodies administered for passive immunization approaches such as these may thus act either in the brain or peripherally, depending on the specific antibody.

The targeting of either fibrillary or soluble Aβ can be achieved by active or passive immunization as described herein and in PCT publications WO 01162801, WO 01/90182, WO 01/18169, WO 00/77178, WO 00/72880, WO 00/72876, WO 99/60024, and WO 99/27944, each of which is incorporated by reference herein in its entirety. Levels of targeted Aβ, specifically levels of soluble Aβ, can be determined by methods known in the art and disclosed herein (see, U.S. Pat. Nos. 5,766,846; 5,837,672; and 5,593,846, which are each incorporated herein by reference in their entirety).

“Passive immunization” as used herein refers to the administration of immune system modulators, such as antibodies, fragments thereof, or immune cells, e.g., T-cells, B-cells, NK cells, NKT cells, dendritic cells, macrophages, basophils, monocytes; or components of the complement pathway, to an individual in order to confer immunity. Components of the complement pathway can be conjugated to proteins to enhance the innate immune response in combination with active or passive immunization. Immunoglobulins (Ig) obtained from human blood may contain antibodies to a variety of agents depending on the pool of human plasma used in preparation. Specific immunoglobulins are obtained from plasma from donors with high levels of antibodies to specific antigens, or donors immunized to produce such a response (Immunization, Cecil Textbook of Medicine, 19^{th} ed. Vol. 1, W.B. Saunders Company 1992; Harrison's Principles of Internal Medicine, 14^{th} ed., McGraw Hill, 1998). Humanized monoclonal antibodies to sequester amyloid-β peptide in plasma, brain, and cerebrospinal fluid to prevent accumulation of the amyloid-β peptide within the brain and the cerebrovasculature are described in WO 01/62801, which is incorporated herein by reference. EP 0613007 describes antibodies having specificity for β-amyloid peptide that is predominantly in a β-sheet conformation. Such antibodies are useful for the invention as described herein.

In one aspect, passive immunization against Aβ is effectuated by transference of an immune cell, i.e., a primed lymphocyte such as a B or T cell, from a second mammal actively immunized against Aβ as described, to a first mammal thereby ameliorating or preventing Aβ related disorders in the first mammal. In another aspect, passive immunization against Aβ is effectuated by transference of an antibody or fragment thereof capable of binding specifically to Aβ. In yet another aspect a human, humanized or chimeric antibody is administered to the first mammal thereby ameliorating or preventing Aβ related disorders. In one embodiment, antibodies which may be administerized for passive immunization include those described in, for example, WO 02/088307, WO 01/62801, EP 0613007, U.S. Pat. No. 5,721,130, U.S. Patent Application Serial No. 20020102261, U.S. Patent Application Serial No. 20020098173, WO 02/41842, U.S. Pat. No. 6,387,674, and EP 1172378, the entire contents of which are incorporated herein by reference.

Thus, in an embodiment of the invention, a vaccine for preventing or treating an amyloid β-related disease in a subject comprises at least one antibody or fragment thereof which interacts with amyloid β to prevent fibrillogenesis, wherein the antibodies are raised against an antigenic amount of a peptide of Aβ, e.g. β sheet region, GAG-binding site region, Aβ (1-42), N-terminal region (1-10), C-terminal region (35-43), and macrophage adherence region Aβ (10-16}, immunogenic fragments thereof, protein conjugates thereof, immunogenic derivative peptides thereof, immunogenic peptides thereof, and immunogenic peptidomimetics thereof, or a peptide which has a substantial identity to any of the above peptides.

Induction of an immune response can be passive, as discussed above, when an antibody or immune system cell (see above) is administered that itself birds to amyloid in a subject, or active, when an immunogen is administered to induce antibodies or T-cells reactive with the immunogen in a subject. Exemplary peptides for inducing or producing an active immune response against β-amyloid protein are described below.

A therapeutic peptide for use in vaccination methods can be an active fragment or analog of a naturally occurring or mutant Aβ peptide that contains an epitope that induces a protective or therapeutic immune response on administration. Immunogenic fragments typically have a sequence of, for example, at least 3, 5, 6, 10, or 20 contiguous amino acids from a natural peptide, although such fragments can include substitutions, as is understood by those of skill in this art. In one embodiment, the compounds are selected from the full-length Aβ peptide, Aβ (1-42), Aβ (1-43), and its lower homologues such as Aβ (1-40), Aβ (1-35), Aβ (1-28), and Aβ (10-21). In another embodiment; the compounds are selected from a group of short peptides, e.g., Aβ (1-5), Aβ (1-6), Aβ (1-7), Aβ (1-10), Aβ (3-7), Aβ (1-3), Aβ (1-12), Aβ (13-28), Aβ (12-28), Aβ (32-42), Aβ (25-35), Aβ (35-40), Aβ (10-16), Aβ (13-21), Aβ (16-21), Aβ (36-42), Aβ (10-22), Aβ (13-22), Aβ (16-22), Aβ (35-42), and Aβ (35-43). The peptides can be shortened further by removing one or more residues from either end or both ends. The peptides can also be modified by replacing one or more residues to modify the properties of the peptides.

The compounds may be all-L peptides, all-D peptides, or peptides comprising a mixture of L and D residues. The compounds may be derived from the peptides above by substitution of one or more residues in the sequence with other amino acid residues or non-amino acid fragments, such as an amino alkanesulfonic acid residue. Peptides may be cyclized or linked to N- or C-terminal modifying groups such as those described in U.S. Pat. No. 5,817,626, U.S. Pat. No. 6,319,498, U.S. Pat. No. 6,303,567, U.S. Pat. No. 5,985,242, U.S. Pat. No. 6,277,826, and WO 00/52048, the contents of which are incorporated herein by reference.

Fragments lacking at least one, and sometimes at least 5 or 10 N- or C-terminal amino acids present in naturally occurring forms of the peptide are used in some embodiments. For example, a fragment lacking 5 amino acids from the C-terminal end of Aβ43, includes the first 38 amino acids from the N-terminal end of Aβ. Fragments from the N-terminal half of Aβ are used in some embodiments. In some embodiments, fragments from the central, β-sheet forming region of Aβ (e.g., Aβ16-20 and Aβ16-21) are used. In some embodiments, Aβ peptide analogs are used. Analogs include allelic, species, and induced variants. Analogs typically differ from naturally occurring peptides at one or a few positions, often by virtue of conservative substitutions. Analogs typically exhibit at least 80 or 90% sequence identity with natural peptides. Some analogs also include unnatural amino acids or modifications of N- or C-terminal amino acids. Examples of unnatural amino acids are alpha, alpha-disubstituted amino acids, N-alkyl amino acids, lactic acid, 4-hydroxyproline, γ-carboxyglutamate, γ-N,N,N-trimethyllysine, γ-N-acetyllysine, O-phosphoserine, N-acetylserine, N-formylmethionine, 3-methylhistidine, 5-hydroxylysine, and ω-N-methylarginine.

Therapeutic peptides may also be composed of longer polypeptides that include, for example, the active peptide amyloid fragment or analog, together with other amino acids. For example, Aβ peptide can be present as intact APP protein or a segment thereof, such as the C-100 fragment that begins at the N-terminus of Aβ and continues to the end of APP. Such polypeptides can be screened for prophylactic or therapeutic efficacy in, e.g., animal models. The Aβ peptide, analog, active fragment, or other polypeptide can be administered in associated form (i.e., as an amyloid peptide) or in dissociated form, such as oligomeric, monomeric, or soluble form. Therapeutic peptides may also include multimers of monomeric and oligomeric immunogenic peptides or conjugates or carrier proteins, or, as mentioned above, maybe added to other fibril components, in order to provide a broader range of anti-amyloid plaque activity.

Other amino acid sequences can include those having adjuvant properties or immunostimulant properties and those that serve to increase the stability of the peptide. For example, in one embodiment peptides include fusion proteins comprising a segment of Aβ fused to a heterologous amino acid sequence that induces a helper T-cell response against the heterologous amino acid sequence and thereby a B-cell response against the Aβ segment Such polypeptides can be screened for prophylactic or therapeutic efficacy in animal models in comparison with untreated or placebo controls. Additional examples include fusions with tetanus toxoid or the Pan DR epitope (PADRE). The Aβ peptide, analog,.active fragment, or other polypeptide can be administered in associated, multimeric form, dendrimeric form, or dissociated form. Therapeutic peptides also include multimers of monomeric and oligomeric immunogenic peptides. More generally, therapeutic peptides for use in the present invention produce or induce an immune response against an amyloid protein. Antibodies may also bind the soluble form in the periphery to act as a sink and drive Aβ from the brain into the periphery, facilitating the clearance of Aβ from the brain by either modulating Aβ equilibrium from the CNS to the periphery or forming a stable complex with Aβ in the periphery. Such peptides therefore include, but are not limited to, the component itself and variants thereof, analogs, and mimetics of the component that induce or cross-react with antibodies to the component, as well as antibodies or T-cells that are specifically reactive with the amyloid peptide.

In a further variation, an immunogenic peptide, such as a fragment of Aβ, can be presented by a vector such as a virus or a bacterium as part of an immunogenic composition. A nucleic acid encoding the immunogenic peptide is incorporated into a genome or episome of the virus or bacteria. Optionally, the nucleic acid is incorporated in such a manner that the immunogenic peptide is expressed as a secreted protein or as a fusion protein with an outer surface protein of a virus or a transmembrane protein of a bacterium so that the peptide is displayed. Viruses or bacteria used in such methods should be nonpathogenic or attenuated. Suitable viruses include adenovirus, herpes simplex virus (HSV), Venezuelan equine encephalitis virus and other alpha viruses, vesicular stomatitis virus, and other rhabdo viruses, vaccinia, and fowl pox. Suitable bacteria include Salmonella and Shigella. Fusion of an immunogenic peptide to HBsAg of HBV is particularly suitable.

Therapeutic peptides also include peptides and other compounds that do not necessarily have a significant amino acid sequence similarity with Aβ but nevertheless serve as mimetics of Aβ and induce a similar immune response. For example, any peptides and proteins forming β-pleated sheets can be screened for suitability. Anti-idiotypic antibodies against monoclonal antibodies to Aβ or other amyloidogenic peptides can also be used. Such anti-Id antibodies mimic the antigen and generate an immune response to it (see Essential Immunology (Roit ed., Blackwell Scientific Publications, Palo Alto, 6^{th} ed.), p. 181). Peptides other than Aβ peptides should induce an immunogenic response against one or more of the segments of Aβ listed above (e.g., 10-16, 10-21, 13-21, 16-21, and 25-35). Preferably, such peptides induce an immunogenic response that is specifically directed to one of these segments without being directed to other segments of Aβ.

In a further embodiment, the compounds may be coupled with a carrier that will modulate the biodistribution, immunogenic property, or the half-life of the compounds. In one embodiment, the therapeutic peptides are those described herein. In other embodiments, the therapeutic peptides are those described in the following references, the entire contents of which are hereby incorporated by reference: WO 00/72880, US 2003-0087407, U.S. Pat. No. 6,462,171, U.S. Patent Application Serial No. 2002-0077288, WO 01/90182, U.S. Pat. No. 5948763, WO 98/44955, WO 01/42306, WO 01/90182, U.S. Patent Application Serial No. 2002-007728, WO 98/08868, WO 96/28471, WO 97121728, WO 95108999, WO 00/68263, U.S. Patent Application Serial No. 2002-0103134 A1, WO 98/30229, U.S. Pat. No. 6,303,567, WO 00/52048, U.S. Pat. No. 6319498, U.S. Pat. No. 6277826, U.S. Pat. No. 5,985,242, U.S. Pat. No. 5,854,215, U.S. Pat. No. 5,854,204, U.S. Pat. No. 5,817,626, WO 96/28471, WO 01/39796, WO 02/096937, WO 96/39834, WO 02/211 41, U.S. Pat. No. 5,688,651, WO 01/18169, WO 99/27949, WO 01/53457, and U.S. Patent Application Serial No. 2002-0052311.

Suitable pharmaceutically acceptable carriers include, without limitation, any non-immunogenic pharmaceutical adjuvants suitable for oral, parenteral, nasal, mucosal, transdermal, intracerebral, intravascular (IV), intraarterial (IA), intramuscular (IM), and subcutaneous (SC) administration routes, as well as carriers suitable for administration through the cerebrospinal fluid. For example, a carrier such as phosphate buffer saline (PBS) can be used.

The pharmaceutical carriers may contain a vehicle that carries antigens to antigen-presenting cells. Examples of vehicles are liposomes, immune-stimulating complexes, microfluidized squalene-in-water emulsions, and microspheres which may be composed of poly(lactic/glycolic) acid (PLGA). Particulates of defined dimensions (<5 micron) include, without limitation, oil-in-water microemulsion (MF59) and polymeric microparticules.

The carriers used in the present invention may also include chemical and genetic adjuvants or immunostimulants to augment immune responses or to increase the antigenicity of immunogens. These adjuvants or immunostimulants exert their immunomodulatory properties through several mechanisms such as lymphoid cell recruitment, cytokine induction, and the facilitation of DNA entry into cells. Cytokine adjuvants include, without limitation, granulocyte-macrophage colony-stimulating factor, interleukin-12, GM-CSF, synthetic muramyl dipeptide analog, or monophosphoryl lipid A. Other chemical adjuvants or immunostimulants include, without limitation, lactic acid bacteria, A1(OH)₃, muramyl dipeptides, and saponins (e.g., QS21). Examples of microbial adjuvants include, without limitation, CpG motifs, Freund's, muramyl dipeptide, LPS derivatives, heat shock protein (HSP), lipid A derivative, polysaccharides, cholera toxin, killed Bordetella pertussis, and LT (lymphotoxin E. coli). Examples of non-microbial adjuvants include, without limitation, aluminum salt, alum, mineral oil, iscoms, liposomes, virosomes, archaeosomes, transfersomes, niosomes, cochleates, proteosomes, calcium phosphate, DDA (dimethyldioctadecylammonium bromide), cytokines, hormones, and C3d. Further, adjuvants can be potentiated by various mutations that augment their activities. Such adjuvants are well known in the art.

The peptide may be coupled to a carrier that will modulate the half-life of the circulating peptide. This will allow control of the period of protection. The peptide-carrier may also be emulsified in an adjuvant and administrated by a standard immunization route.

The vaccines of the present invention will, for the most part, be administered parenterally, such as intravascularly (IV), intraarterially (IA), intramuscularly (IM), subcutaneously (SC), transdermally, or the like. In some instances, administration may be oral, nasal, rectal, transdermal, or by aerosol, where the nature of the vaccine allows for transfer to the vascular system. Usually a single injection will be employed although more than one injection may be used, if desired. Optionally, a primary immunization can be followed by one or more boosts with an interval of, e.g., a few weeks, using the same antigen or a further modified antigen if desired. The route may be the same or different for the primary immunization and boost(s). Further, the adjuvants or immunostimulants may also be identical or different if desired. The vaccine may be administered by any convenient means, including syringe, trocar, catheter, or the like. Preferably, the administration will be intravascular, where the site of introduction is not critical to this invention, preferably at a site where there is rapid blood flow, e.g. intravenously, in a peripheral or central vein. Other routes may find use where the administration is coupled with slow release techniques or a protective matrix.

An “immunogenic peptide” or “immunogen” or “antigen” is a molecule that is capable of inducing an immunological response against itself upon administration to a subject, either in conjunction with, or in the absence of, an adjuvant. Such molecules include, for example, amyloidogenic peptides or fragments thereof conjugated to a carrier protein, such as keyhole limpet hemocyanin (KLH), C3d, polysaccharide, bovine serum albumin (BSA), tetanus toxoid (e.g., TT830-844 or TT947-967), heat shock protein (HSP, e.g., HSP65), ovalbumin, or cholera toxin. See, eg., WO 00/72876 for examples of additional adjuvants that can be used in the present invention, as well as examples of fusion molecules comprising Aβ sequences and adjuvant sequences, such as tetanus toxoid sequences.

The term “antibody” or “immunoglobulin” is used to include intact antibodies and fragments thereof. An antibody can be a monoclonal or polyclonal antibody and can be made by recombinant techniques, collected from serum or ascites, or from hybridoma sources. Typically, fragments compete with the intact antibody from which they were derived for specific binding to an antigen fragment. Fragments are produced by recombinant DNA techniques, or by enzymatic or chemical cleavage of intact antibodies. Antigen binding fragments include Fab, Fab′, F(ab′)₂, Fv, and single-chain antibodies, further including separate heavy chains or light chains. Antibody fragments are produced by recombinant DNA techniques, or by enzymatic or chemical separation of intact immunoglobulins. The term “antibody” encompasses one or more immunoglobulin chains that are chemically conjugated to, or expressed as, fusion proteins with other proteins. The term “antibody” also includes bispecific antibodies. A bispecific or bifunctional antibody is an artificial hybrid antibody having two different heavy/light chain pairs and two different binding sites. Bispecific antibodies can be produced by a variety of methods including fusion of hybridomas or linking of Fab′ fragments. See, e.g., Songsivilai et al., Clin. Exp. Immunol. 79, 315-321 (1990); Kostelny et al., J. Immunol. 148, 1547-1553 (1992).

The term “immunological” or “immune” or “immunogenic” response refers to the development of a humoral (antibody mediated) or a cellular (mediated by antigen-specific T cells or their secretion products) response directed against an antigen in a vertebrate individual. Such a response can be an active response induced by administration of immunogen or a passive response induced by administration of antibodies or immune cells such as primed T-cells, B cells, macrophages NK or NKT cells, or primed dendritic cells that can act as antigen presenting cells. A cellular immune response is elicited by the presentation of polypeptide epitopes in association with Class I or Class II MHC molecules to activate antigen-specific CD4⁺ T helper cells or CD8⁺ cytotoxic T cells. The response may also involve activation of immune cells, such as monocytes, macrophages, NK cells, basophils; dendritic cells, astrocytes, microglia cells, eosinophils or other components of innate immunity. The presence of a cell-mediated immunological response can be determined by standard proliferation assays (CD4⁺ T cells) or CTL (cytotoxic T lymphocyte) assays known in the art. The relative contributions of humoral and cellular responses to the protective or therapeutic effect of an immunogen can be distinguished by separately isolating immunoglobulin (IgG) and T-cell fractions from an immunized first mammal and measuring a protective or therapeutic effect in a second subject.

The terms “polynucleotide” and “nucleic acid,” as used interchangeably herein refer to a polymeric molecule having a backbone that supports bases capable of hydrogen bonding to typical polynucleotides, where the polymer backbone presents the bases in a manner to permit such hydrogen bonding in a sequence specific fashion between the polymeric molecule and a typical polynucleotide (e.g., single-stranded DNA). Such bases are typically inosine, adenosine, guanosine, cytosine, uracil, and thymidine. Polymeric molecules include double and single stranded RNA and DNA, and backbone modifications thereof, for example, methylphosphonate linkages. The term “polypeptide” as used herein refers to a compound made up of a single chain of amino acid residues linked by peptide bonds. The term “protein” may be synonymous with the term “polypeptide” or may refer to a complex of two or more polypeptides. The term “peptide” also refers to a compound composed of amino acid residues linked by peptide bonds. Generally peptides are composed of 100 or fewer amino acids, while polypeptides or proteins have more than 100 amino acids. As used herein, the term “protein fragment” may also be read to mean a peptide.

It is appreciated that immunological responses directed at other amyloid plaque components can also be effective in preventing, retarding, or reducing plaque deposition in amyloid diseases. Such components may be minor components of fibrils or associated with fibrils or fibril formation in the plaques, with the caveat that components that are ubiquitous throughout the body, or relatively non-specific to the amyloid deposit, are generally less suitable for use as therapeutic targets.

Specific examples of peptides that can be used as vaccine antigens in the present invention include those described in WO 02/096937 A2, which are within the following formula:
R′—(P)—R″ (I),
where P is an all-D peptide of an amyloid protein, e.g., β sheet region, GAG-binding site region, Aβ (1-42, all-D), and macrophage adherence region (10-16, all-D), immunogenic fragments thereof, immunogenic derivatives thereof, protein conjugates thereof, immunogenic peptides thereof, and immunogenic peptidomimetics thereof; R′ is an N-terminal substituent, e.g., hydrogen; lower alkyl groups, e.g., acyclic or cyclic having 1 to 8 carbon atoms, without or with functional groups, e.g., carboxylate, sulfonate and phosphonate; aromatic groups; heterocyclic groups; and acyl groups, e.g., alkylcarbonyl, arylcarbonyl, sulfonyl and phosphonyl groups; and R″ is a C-terminal substituent, e.g., hydroxy, alkoxy, aryloxy, unsubstituted or substituted amino groups. In one example, R′ and R″ are identical or different, and the alkyl or aryl groups of R′ and R″ are further substituted with functionalities such as halide (e.g. F, Cl, Br, or I), hydroxyl, alkoxyl, aryloxyl, hydroxycarbonyl, alkoxy!carbonyl, aryloxycarbonyl, carbamyl, unsubstituted or substituted amino, sulfo or alkyloxysulfonyl, phosphono or alkoxyphosphonyl groups. When the compound has an acid functional group, it can be in the form of a pharmaceutically acceptable salt or ester. When the compound has a basic functional group, it can be in the form of a pharmaceutically acceptable salt.

Specific examples of peptides from WO 021096937 A2 that can be used in the present invention include the following:

Aβ (1-42, all-D) DAEFRHDSGYEVHHQKLVFFAEDVGSNKGAIIGL
MVGGVVIA,
Aβ (1-40, all-D) DAEFRHDSGYEVHHQKLVFFAEDVGSNKGAIIGL
MVGGVV,
Aβ (1-35, all-D) DAEFRHDSGYEVHHQKLVFFAEDVGSNKGAIIGL
M,
Aβ (1-28, all-D) DAEFRHDSGYEVHHQKLVFFAEDVGSNK,
Aβ (1-7, all-D) DAEFRHD,
Aβ (10-16, all-D) YEVHHQK,
Aβ (16-21, all-D) KLVFFA,
Aβ (10-21, all-D) YEVHHQKLVFFA,
Aβ (13-21, all-D) HHQKLVFFA,
Aβ (36-42, all-D) VGGVVIA,
as well as
Lys-Ile-Val-Phe-Phe-Ala (all-D); Lys-Lys-Leu-Val-
Phe-Phe-Ala (all-D); Lys-Phe-Val-Phe-Phe-Ala
(all-D); Ala-Phe-Phe-Val-Leu-Lys (all-D); Lys-Leu-
Val-Phe (all-D); Lys-Ala-Val-Phe-Phe-Ala (all-D);
Lys-Leu-Val-Phe-Phe (all-D); Lys-Leu-Val-Phe-Phe-
Ala-NH₂ (all-D); Lys-Ile-Val-Phe-Phe-Ala-NH₂
(all-D); Lys-Leu-Val-Phe-Phe-Ala-NH₂ (all-D); Lys-
Phe-Val-Phe-Phe-Ala-NH₂ (all-D); Ala-Phe-Phe-Val-
Leu-Lys-NH₂ (all-D); Lys-Leu-Val-Phe-NH₂ (all-D);
Lys-Ala-Val-Phe-Phe-Ala-NH₂ (all-D); Lys-Leu-Val-
Phe-Phe-NH₂ (all-D); Lys-Val-Val-Phe-Phe-Ala-NH₂
(all-D); Lys-Leu-Val-Phe-Phe-Ala-Glu (all-D); Lys-
Leu-Val-Phe-Phe-Ala-Glu-NH₂ (all-D); His-His-Gln-
Lys-Leu-Val-Phe-Phe-Ala-Glu (all-D); Asp-Asp-Asp
(all-D); Lys-Val-Asp-Asp-Gln-Asp (all-D); His-His-
Gln-Lys (all-D); Phe-Phe-NH—CH₂CH₂SO₃H (all-D);
Phe-Phe-NH—CH₂CH₂CH₂SO₃H (all-D); Phe-Phe-
NH—CH₂CH₂CH₂CH₂SO₃H (all-D); Phe-Tyr-NH—CH₂CH₂SO₃H
(all-D); Phe-Tyr-NH—CH₂CH₂CH₂SO₃H (all-D); Phe-
Tyr-NH—CH₂CH₂CH₂CH₂SO₃H (all-D); HO₃SCH₂CH₂-Phe-
Phe (all-D); HO₃SCH₂CH₂CH₂-Phe-Phe (all-D);
HO₃SCH₂CH₂CH₂CH₂-Phe-Phe (all-D); HO₃SCH₂CH₂-Phe-
Tyr (all-D); HO₃SCH₂CH₂CH₂-Phe-Tyr (all-D);
HO₃SCH₂CH₂CH₂CH₂-Phe-Tyr (all-D); HO₃SCH₂CH₂-Leu-
Val-Phe-Phe-Ala (all-D); HO₃SCH₂CH₂CH₂-Leu-Val-
Phe-Phe-Ala (alI-D); HO₃SCH₂CH₂CH₂CH₂-Leu-Val-Phe-
Phe-Ala (all-D); Leu-Val-Phe-Phe-Ala-NH—CH₂CH₂SO₃H
(all-D); Leu-Val-Phe-Phe-Ala-NH—CH₂CH₂CH₂SO₃H
(all-D); Leu-Val-Phe-Phe-Ala-NH—CH₂CH₂CH₂CH₂SO₃H
(all-D); Aβ ([L] 10-15 [D] 16-21); Y[L]-E[L]-V[L]-
H[L]-H[L]-Q[L]-K[D]-L[D]-V[D]-F[D]-F[D]-A[D]; Aβ
([D] 16-21 [L] 22-28); K[D]-His-His-Gln-Lys-Leu-
Val-Phe-Phe-Ala-Glu-ASp-Val (all-D); Lys-Leu-Val-
Phe-Phe-Ala-Glu-Asp-Val-Gly-Ser-Asn-Lys (all-D);
and
Arg-His-Asp-Ser-Gly-Val-His-His-Gln-Lys-Leu-Val-
Phe-Phe-Ala (all-D).

The compounds listed above may be modified by removing or inserting one or more amino acid residues, or by substituting one or more amino acid residues with other amino acid or non-amino acid moieties or fragments. As specific examples, the following are compounds derived from all-D KLVFFA-NH₂, noted above, by substituting one or two amino acid residue(s) with other amino acids: Lys-Leu-Val-Trp-Phe-Ala-NH₂ (all-D); Lys-Leu-Val-Phe-Trp-Ala-NH₂ (all-D); Lys-Leu-Val-Trp-Trp-Ala-NH₂ (all-D); Lys-Leu-Val-Tyr-Phe-Ala NH₂ (all-D); Lys-Leu-Val-Phe-Tyr-Ala-NH₂ (all-D); Lys-Leu-Val-Tyr-Tyr-Ala-NH₂ (all-D); Lys-Leu-Val-Thi-Phe-Ala-NH₂ (all-D); Lys-Leu-Val-Phe-Thi-Ala-NH₂ (all-D); Lys-Leu-Val-Thi-Thi-Ala-NH₂ (all-D); Lys-Leu-Val-Cha-Phe-Ala-NH₂ (all-D); Lys-Leu-Val-Phe-Cha-Ala-NH₂ (all-D); Lys-Leu-Val-Cha-Cha-Ala-NH₂ (all-D); Lys-Leu-Val-Pgly-Phe-Ala-NH₂ (all-D); Lys-Leu-Val-Phe-Pgly-Ala-NH₂ (all-D); and Lys-Leu-Val-Pgly-Pgly-Ala-NH₂ (all-D). For these compounds, the terms Thi, Cha, and Pgly are intended to mean thienylalanine, cyclohexylalanine, and phenylglycine, respectively.

As used with respect to the above-noted compounds, “all-D” includes peptides having at least 50% D-configuration amino acids. Preferably, “all-D” also includes peptides having greater than or equal to 50%; 55%;-60%; 65%; 70%; 75%; 80%; 85%; 90%; 95% or 100% D-configuration amino acids.

In addition to being used in active vaccination methods, as described above, therapeutic peptides may also act directly as anti-fibrillogenic agents with or without eliciting an immune response. For example, a therapeutic peptide may bind to Aβ to prevent or inhibit its fibril formation. As a specific example, the 16-21 region of the Aβ peptide, KLVFPA, is responsible for the β-sheet formation and the intermolecular interactions of Aβ during fibrillogenesis. Peptides from this region have been extensively tested for their antifibrillogenic activity Tjernberg et al., J. Biol. Chem. 272, 12601-12605 (1997); Findeis et al., Biochemistry 38, 6791-6800 (1999)). Hexapeptides, for example, can bind to Aβ and maintain the protein in a non-aggregated state. It has also been shown that, for example, Cholyl-LVFFA-OH can act as an anti-fibrillogenic agent (Findeis et al., Amyloid, 231-241 (2001)). Any of the peptides described herein may be used as an anti-fibrillogenic agent in this way. In one embodiment, L- or D-peptides, or peptides possessing mixtures of L- and D-amino acids, may be used in this approach, or modified or substituted peptides, as defined above, may be used. Further, the term “all D” as used with respect to the following peptides is as described above.

Examples of therapeutic peptides that can be used as second agents in the present invention are described, for example, in WO 00/68263. This publication describes antifibrillogenic agents for inhibiting amyloidosis that comprises a peptide of Formula I, an isomer thereof, a retro or a retro-inverso isomer thereof or a peptidomimetic thereof:
Xaa₁-Xaa₂-Xaa₃-Xaa₄ I
where, Xaa, is absent or selected from the group consisting of Lys, Lys-Lys, Xaa₅-Lys-, and Ala; Xaa₅ is absent or selected from the group consisting of His-Gln-, His-His-Gln-, Val-His-His-Gln-, Glu-Val-His-His-Gln-, Asp-Asp-Asp-, Lys-Val-Asp-Asp-Gln-Asp-, Gln-; Xaa₂ is absent or any amino acid; Xaa₃ is absent, Val or Phe; Xaa₄ is absent or selected from the group consisting of Phe, Phe-NH₂, Phe-Phe, Phe-Phe-Ala, Phe-Phe-Ala-NH₂, Phe-Phe-Ala-Gln, Phe-Phe-Ala-Gln-NH₂, Val-Leu-Lys, Val-Leu-Lys-NH₂; where the peptide of formula I contains at least one Lys or Asp; and where the peptide has at least one [D] amino acid residue. Preferably, Xaa₂ is a hydrophobic amino acid residue such as a leucine residue.

In one example, the peptide of formula I has at least two [D] amino acid residues, and more preferably at least three [D] amino acid residues. Optionally, the peptide of formula I has one [L] amino acid residue, or more preferably the peptide is an all-[D] isomer peptide.

In other examples, the peptide of Formula I is selected from, the group consisting of:

Lys-Ile-Val-Phe-Phe-Ala; Lys-Lys-Leu-Val-Phe-Phe-
Ala; Lys-Leu-Val-Phe-Phe-Ala; Lys-Phe-Val-Phe-Phe-
Ala; Ala-Phe-Phe-Val-Leu-Lys; Lys-Leu-Val-Phe;
Lys-Ala-Val-Phe-Phe-Ala; Lys-Leu-Val-Phe-Phe; Lys-
Val-Val-Phe-Phe-Ala; Lys-Ile-Val-Phe-Phe-Ala-NH₂;
Lys-Leu-Val-Phe-Phe-Ala-NH₂; Lys-Phe-Val-Phe-Phe-
Ala-NH₂; Ala-Phe-Phe-Val-Leu-Lys-NH₂; Lys-Leu-Val-
Phe-NH₂; Lys-Ala-Val-Phe-Phe-Ala-NH₂; Lys-Leu-Val-
Phe-Phe-NH₂; Lys-Val-Val-Phe-Phe-Ala-NH₂; Lys-Leu-
Val-Phe-Phe-Ala-Gln; Lys-Leu-Val-Phe-Phe-Ala-Gln-
NH₂; His-His-Gln-Lys-Leu-Val-Phe-Phe-Ala-NH₂; Asp-
Asp-Asp; Lys-Val-Asp-Asp-Gln-Asp; His-His-Gln-Lys;
and
Gln-Lys-Leu-Val-Phe-Phe-NH₂.

The term “agents having stereoselective antifibrillogenic activity” is intended to mean any peptides, peptide analogues, peptide derivatives, or peptidomimetics which retain the stereoselective antifibrillogenic activity, the cytoprotective and anti-inflammatoy activity and/or the ability to alter a natural amyloidotic protein aggregation as described herein. Peptide analogues, peptide derivatives, or peptidomimetics include any molecules that mimic the chemical structure of a peptide and retain the functional properties of the peptide (Williams et al., eds., Biologically Active Peptides: Design, Synthesis, and Utilization, vol. 1, Technomic Publishing Company Inc., Lancaster, Pa., 1993, page 35). Examples of peptide analogues, peptide derivatives, or peptidomimetics include compounds with sulfonamide, phosphoramide, or non-amide linkages.

The expression “antifibrillogenic activity,” as used with respect to the above-described second agents, is intended to mean the ability to block or prevent an amyloidogenic protein from forming fibrils, preferably by preventing it from adopting its β-pleated conformation. The expression “retro isomer” is intended to mean a reversal of the direction of the peptide backbone. The expression “inverso isomer” is intended to mean an inversion of the amino acid chirality used to make the peptide. The expression “retro-inverso isomer” is intended to mean a reversal of both the peptide backbone direction and the amino acid chirality.

Examples of peptides that can be used as the second agent in the methods and compositions of the invention are also described in, for example, U.S. Pat. Nos, 5,817,626, 5,854,215, 5,854,204, 6,303,567, 6,319,498, 5,985,242, and 6,277,826, and in U.S. Patent Application Publication Nos. 20020103134 and 200200981 73. Examples of peptides from these publications that can be used in the invention include, for example, those that are within the following formula, with or without the indicated modifying groups:

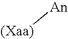

where Xaa is a β-amyloid peptide, A is a modulating group attached directly or indirectly to the β-amyloid peptide of the compound such that the compound inhibits aggregation of natural β-amyloid when contacted with the natural β-amyloid peptides, and n is an integer selected such that the compound inhibits aggregation of natural β-amyloid peptides when contacted with the natural β-amyloid peptides. In one example, a β-amyloid peptide of the compounds of this formula has an amino-terminal amino acid residue corresponding to position 668 of β-amyloid precursor protein-770 (APP-770) or to a residue carboxyl-terminal to position 668 of APP-770. The amino acid sequence of APP-770 from position 668 to position 770 (i.e., the carboxyl terminus) is shown below:

EVKMDAEFRHDSGYEVHHQKLVFFAEDVGSNKGAIIGLMVGGVVIATVIV
ITLVMLKKKQYTSIHHGVVEVDAAVTPEERHLSKMQQNGYENPTYKFFEQ
MQN.

In another example, the amino-terminal amino acid residue of the β-amyloid peptide corresponds to position 672 of APP-770 (position 5 of the amino acid sequence noted above) or to a residue carboxyl-terminal to position 672 of APP-770. Although the β-amyloid peptide of the compound may encompass the 103 amino acid residues corresponding to positions 668-770 of APP-770, preferably the peptide is between 6 and 60 amino acids in length, e.g., between 10 and 43 amino acids in length or between 10 and 25 amino acid residues in length.

As used herein, the term “β-amyloid peptide,” as used with respect to a modulator as described herein is intended to encompass peptides having an amino acid sequence identical to a portion of that of the natural sequence in APP, as well as peptides having acceptable amino acid substitutions from the natural sequence. Acceptable amino acid substitutions are those that do not affect the ability of the peptide to alter natural β-amyloid peptide (β-AP) aggregation. Moreover, particular amino acid substitutions may further contribute to the ability of the peptide to alter natural β-AP aggregation and/or may confer additional beneficial properties on the peptide (e.g., increased solubility, reduced association with other amyloid proteins, etc.). For example, substitution of hydrophobic amino acid residues for the two phenylalanine residues at positions 19 and 20 of natural β-AP may further contribute to the ability of the peptide to alter β-AP AP aggregation (see Hilbich, J. Mol. Biol. 228, 460473 (1992)). Thus, in one example, the β-AP AP of the compound consists of the amino acid sequence shown below: DAEFRHDSGYEVHHQKLV(Xaa₁₉)(Xaa₂₀)AEDVGSNKGAIIGLMVGGVVIAT (or an amino-terminal or carboxy-terminal deletion thereof), wherein Xaa is a hydrophobic amino acid. Examples of hydrophobic amino acids are isoleucine, leucine, threonine, serine, alanine, valine, and glycine. In one specific embodiment, F₁₉F₂₀ is substituted with T₁₉T₂₀ or G₁₉I₂₀.

Other suitable amino acid substitutions include replacement of amino acids in the human peptide with the corresponding amino acids of the rodent β-AP peptide. The three amino acid residues that differ between human and rat β-AP are at positions 5, 10, and 13 of the amino acid sequences DAEFRHDSGYEVHHQKLVFFAEDVGSNKGARGLMVGGVVIAT and DAEFRHDSGYEVHHQKLV(Xaa₁₉)(Xaa₂₀)AEDVGSNKGAIIGLMVGGVVIAT. A human β-AP having the human to rodent substitutions Arg5 to Gly, Tyr₁₀ to Phe, and His₁₃ to Arg has been shown to retain the properties of the human peptide (see Fraser et al., Biochemistry 31, 10716-10723 (1992); and Hilbich et al., Eur. J. Biochem. 201, 61-69 (1991)). Accordingly, a human β-AP having rodent β-AP amino acid substitutions is suitable for use in a modulator used in this invention.

Other possible β-AP amino acid substitutions are described in Hilbich et al., J. Mol. Biol. 218, 149-163 (1991); and Hilbich, J. Mol. Biol. 228, 460-473 (1992). Moreover, amino acid substitutions that affect the ability of β-AP to associate with other proteins can be introduced. For example, one or more amino acid substitutions that reduce the ability of β-AP to associate with the serpin enzyme complex (SEC) receptor, α1-antichymotrypsin (ACT), and/or apolipoprotein E (ApoE) can be introduced. A preferred substitution for reducing binding to the SEC receptor is L₃₄ M₃₅ to A₃₄ A₃₅ (at positions 34 and 35 of the amino acid sequences shown above). A preferred substitution for reducing binding to ACT is S₈ to A₈ (at position 8 of the amino acid sequences shown above).

Alternative to β-AP amino acid substitutions described herein or known in the art, a modulator composed, at least in part of an amino acid-substituted β-amyloid peptide can be prepared by standard techniques and tested for the ability to alter β-AP aggregation using an aggregation assay described herein. To retain the properties of the original modulator, preferably conservative amino acid substitutions are made at one or more amino acid residues. A “conservative amino acid substitution” is one in which the amino acid residue is replaced with an amino acid residue having a similar side chain. Families of amino acid residues having similar side chains have been defined in the art, including basic side chains (e.g., lysine, arginine, and histidine), acidic side chains (e.g., aspartic acid and glutamic acid), uncharged polar side chains (e.g., glycine, asparagine, glutamine, serine, threonine, tyrosine, and cysteine), nonpolar side chains (e.g., alanine, valine, leucine, isoleucine, proline, phenylalanine, methionine, and tryptophan), β-branched side chains (e.g., threonine, valine, and isoleucine) and aromatic side chains (e.g., tyrosine, phenylalanine, tryptophan, and histidine). Accordingly, a modulator composed of a β-amyloid peptide having an amino acid sequence that is mutated from that of the wild-type sequence in APP-770, yet which still retains the ability to alter natural β-AP aggregation can be used in the invention.

As used herein, the term “β-amyloid peptide” is further intended to include peptide analogues or peptide derivatives or peptidomimetics that retain the ability to alter natural β-AP aggregation as described herein. For example, a β-amyloid peptide of a modulator of the invention may be modified to increase its stability, bioavailability,.solubility, etc. The terms “peptide analogue,” “peptide derivative,” and “peptidomimetic” as used herein are intended to include molecules that mimic the chemical structure of a peptide and retain the functional properties of the peptide. Approaches to designing peptide analogs are known in the art. For example, see Farmer, in Drug Design (Ariens, ed.) Academic Press, New York, 1980, vol. 10, pp. 119-143; Ballet al., J. Mol. Recognition 3, 55 (1990); Morgan et al., Ann Rep. Med. Chem. 24, 243 (1989); and Freidinger, Trends Pharmacol. Sci. 10, 270 (1989). Examples of peptide analogues, derivatives, and peptidomimetics include peptides substituted with one or more benzodiazepine molecules (see e.g., James et al., Science 260:1937-1942 (1993)), peptides with methylated amide linkages and “retro-inverso” peptides (see U.S. Pat. No. 4,522,752). Peptide analogues, peptide derivatives and peptidomimetics are described in further detail below with regard to compounds comprising an Aβ aggregation core domain. Any of the above-described modifications can also be used with the vaccine peptides described herein, as determined to be appropriate by those of skill in this art.

In a modulator of the invention having the formula shown above, a modulating group (“A”) can be attached directly or indirectly to the P-amyloid peptide of the modulator. (As used herein, the term “modulating group” and “modifying group” are used interchangeably to describe a chemical group directly or indirectly attached to an Aβ derived peptidic structure.) For example, the modulating group can be directly attached by covalent coupling to the β-amyloid peptide or the modulating group can be attached indirectly by a stable non-covalent association. In one example that can be used in the invention, the modulating group is attached to the amino-terminus of the β-amyloid peptide of the modulator. Accordingly, the modulator can comprise a compound having a formula:

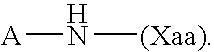

Alternatively, in another example, the modulating group is attached to the carboxyl-terminus of the β-amyloid peptide of the modulator. Accordingly, the modulator can comprise a compound having a formula:

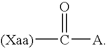

In yet another embodiment, the modulating group is attached to the side chain of at least one amino acid residue of the β-amyloid peptide of the compound (e.g. through the epsilon amino group of a lysyl residue(s), through the carboxyl group of an aspartic acid residue(s) or a glutaric acid residue(s), through a hydroxy group of a tyrosyl residue(s), a serine residue(s) or a threonine residue(s) or other suitable reactive group on an amino acid side chain).

The modulating group is selected such that the compound inhibits aggregation of natural β-amyloid peptides when contacted with the natural β-amyloid peptides. Accordingly, since the β-AP peptide of the compound is modified from its natural state, the modulating group “A” as used herein is not intended to include hydrogen. In a preferred embodiment, the modulating group is a biotin compound of the formula: 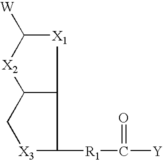
wherein X₁—X₃ are each independently selected from the group consisting of S, 0, and NR₂, wherein R_{2 is} hydrogen, or an aryl, lower alkyl, alkenyl or alkynyl moiety; W is ═O or N(R₂)₂; R₁ is a lower alkylenyl moiety and Y is a direct bond or a spacer molecule selected for its ability to react with a target group on a β-AP. At least one of X₁—X₃ is an NR₂ group or W is an N(R₂)₂ group.

The term “aryl” is intended to include aromatic moieties containing substituted or unsubstituted ring(s), e.g., benzyl, naphthyl, etc. Other more complex fused ring moieties also are intended to be included.

The term “lower alkyl or alkylenyl moiety” refers to a saturated, straight or branched chain (or combination thereof) hydrocarbon containing 1 to about 6 carbon atoms, more preferably from 1 to 3 carbon atoms. The terms “lower alkenyl moiety” and “lower alkynyl moiety” refer to unsaturated hydrocarbons containing 1 to about 6 carbon atoms, more preferably 1 to 3 carbon atoms. Preferably, R₂ contains 1 to 3 carbon atoms. Preferably, R₁ contains 4 carbon atoms.

The spacer molecule (Y) can be, for example, a lower alkyl group or a linker peptide, and is preferably selected for its ability to-link with a free amino group (e.g., the α-amino group at the amino-terminus of a β-AP). Thus, in a preferred embodiment, the biotin compound modifies the amino-terminus of a β-amyloid peptide.

Additional suitable modulating groups may include other cyclic and heterocyclic compounds and other compounds having similar steric “bulk.” Non-limiting examples of compounds that can be used to modify a β-AP include N-acetylneuraminic acid, cholic acid, trans-4-cotininecarboxylic acid, 2-imino-1-imidazolidineacetic acid, (S)(−)-indoline-2-carboxylic acid, (−)menthoxyacetic acid, 2-norbornaneacetic acid, γoxo-5-acenaphthenebutyric acid, (−)-2-oxo-4-thiazolidinecarboxylic acid, tetrahydro-3-furoic acid, 2-iminobiotin-N-hydroxysuccinimide ester, diethylenetriaminepentaacetic dianhydride, 4-morpholinocarbonyl chloride, 2-thiopheneacetyl chloride, 2-thiophenesulfonyl chloride, 5-(and 6-)-carboxyfluorescein (succinimidyl ester), fluorescein isothiocyanate, and acetic acid (or derivatives thereof).

In a modulator that can be used in the invention, a single modulating group may be attached to a β-amyloid peptide (e.g., n=1 in the formula shown above) or multiple modulating groups may be attached to the peptide. The number of modulating groups is selected such that the compound inhibits aggregation of natural β-amyloid peptides when contacted with the natural β-amyloid peptides. However, n preferably is an integer between 1 and 60, more preferably between 1 and 30, and even more preferably between 1 and 10 or 1 and 5.

In another example, a β-amyloid modulator compound of the invention comprises an Aβ aggregation core domain (abbreviated as ACD) coupled directly or indirectly to a modifying group such that the compound modulates the aggregation or inhibits the neurotoxicity of natural ⊕-amyloid peptides when contacted with the natural β-amyloid-peptides. As used herein, an “Aβ aggregation core domain” is intended to refer to a structure that is modeled after a subregion of a natural β-amyloid peptide which is sufficient to modulate aggregation of natural β-APs when this subregion of the natural β-AP is appropriately modified as described herein (e.g., modified at the amino-terminus). The term “subregion of a natural α-amyloid peptide” is intended to include amino-terminal and/or carboxy-terminal deletions of natural β-AP. The term “subregion of natural β-AP” is not intended to include full-length natural β-AP (i.e., “subregion” does not include Aβ₁₋₃₉, Aβ₁₋₄₀, Aβ₁₋₄₁, Aβ₁₋₄₂, and Aβ₁₋₄₃).

Although not-intending to be limited by mechanism, the ACD of the modulators of the invention is thought to confer,a specific targeting function on the compound that allows the compound to recognize and specifically interact with natural β-AP. Preferably, the ACD is modeled after a subregion of natural β-AP that is less than 15 amino acids in length and more preferably is between 3-10 amino acids in length. In various embodiments, the ACD is modeled after a subregion of β-AP that is 10, 9, 8, 7, 6, 5, 4, or 3 amino acids in length. In one embodiment, the subregion of β-AP upon which the ACD is modeled is an internal or carboxy-terminal region of β-AP (Le., downstream of the amino-terminus at amino acid position 1). In another embodiment, the ACD is modeled after a subregion of β-AP that is hydrophobic. In certain specific embodiments, the term Aβ aggregation core domain specifically excludes β-AP subregions corresponding to amino acid positions 1-15 (Aβ₁₋₁₅), 6-20 (Aβ₆₋₂₀), and 16-40 (Aβ₁₆₋₄₀).

An Aβ aggregation core domain can be comprised of amino acid residues linked by peptide bonds. That is, the ACD can be a peptide corresponding to a subregion of β-AP. Alternatively, an Aβ aggregation core domain can be modeled after the natural Aβ peptide region but may be comprised of a peptide analogue, peptide derivative or peptidomimetic compound, or other similar compounds which mimic the structure and function of the natural peptide. Accordingly, as used herein, an “Aβ aggregation core domain” is intended to include peptides, peptide analogues, peptide derivatives and peptidomimetic compounds which, when appropriately modified, retain the aggregation modulatory activity of the modified natural Aβ peptide subregion. Such structures that are designed based upon the amino acid sequence are referred to herein as “Aβ derived peptidic structures.” Approaches to designing peptide analogues, derivatives and mimetics are known in the art. For example, see Farmer, in Drug Design (Ariens, ed.) Academic Press, New York, 1980, vol. 10, pp.119-143; Ball et al., J. Mol. Recognition 3, 55 (1990); Morgan et al., Ann. Rep. Med. Chem. 24, 243 (1989); and Freidinger, Trends Pharmacol. Sci. 10, 270 (1989). See also Sawyer, (1995) “Peptidomimetic Design and Chemical Approaches to Peptide Metabolism” in Taylor et al. (eds.) Peptide-Based Drug Design: Controlling Transport and Metabolism, Chapter 17; Smith, 3^{rd}, et al., J. Am. Chem. Soc. 117, 11113-11123(1995); Smith, 3^{rd}, et al., J. Am. Chem. Soc. 116, 9947-9962(1994); and Hirschman et al., J. Am. Chem. Soc. 115, 12550-12568(1993).

As used herein, a “derivative” of a compound X (eg., a peptide or amino acid) refers to a form of X in which one or more reaction groups on the compound have been derivatized with a substituent group. Examples of peptide derivatives include peptides in which an amino acid side chain, the peptide backbone, or the amino- or carboxy-terminus has been derivatized (e.g., peptidic compounds with methylated amide linkages). As used herein an “analogue” of a compound X refers to a compound that retains chemical structures of X necessary for functional activity of X yet which also contains certain chemical structures that differ from X. An example of an analogue of a naturally-occurring peptide is a peptide which includes one or more non-naturally-occurring amino acids. As used herein, a “mimetic” of a compound X refers to a compound in which chemical structures of X necessary for functional activity of X have been replaced with other chemical structures that mimic the conformation of X. Examples of peptidomimetics include peptidic compounds in which the peptide backbone is substituted with one or more benzodiazepine molecules (see, e.g., James et al., Science 260, 1937-1942 (1993)), peptides in which all L-amino acids are substituted with the corresponding D-amino acids and “retro-inverso” peptides (see U.S. Pat. No. 4,522,752), described further below.

Other peptides that can be used to inhibit amyloid-β fibril formation in the present invention include antisense peptides corresponding to amyloid-β sequences 1-43 or fragments thereof (WO 02136614 A2). Antisense peptides are derived, for example, from the complementary strand of DNA encoding a given protein, read in the same open reading frame. Alternatively, they can be derived from the amino acid sequence of a protein by reverse translation to produce a cDNA. In any case, antisense peptides generally have hydropathy profiles opposite to those of the corresponding sense peptides, thus making them strong candidates for use as agents that bind to and, in the case of amyloid-β peptides, inhibit the fibril formation of, the corresponding-sense peptides.

As is noted above, antisense peptides that can be used in the present invention include those that correspond to amyloid-β sequences 1-43 or fragments thereof. In addition, peptides that have at least 60% (e.g., 70%, 80%, 90%, or 95%) sequence identity to such antisense peptides can be used. Further, antisense peptides can include naturally occurring or synthetic amino acid derivatives, as is well known in the art. Methods for testing a candidate antisense peptide for use in the invention are described, for example, in WO 02/36614 A2, as well as in Milton, Biochem. J. 344, 293-296 (1999). Peptides that bind to the corresponding sense peptides with a dissociation constant (Kd) of less than 50 μm, e.g., less than 10μM, may be selected for use. Specific examples of antisense peptides that can be used in the present invention include the antisense, sequences of amyloid-β 17-24, 31-35, 3-30, 17-35, 12-24, 12-28, 14-35, 25-35, and any other amyloid-β fragments mentioned elsewhere herein.

The term mimetic, and in particular, peptidomimetic, is intended to include isosteres. The term “isostere” as used herein is intended to include a chemical structure that can be substituted for a second chemical structure because the steric conformation of the first structure fits a binding site specific for the second structure. The term specifically includes peptide back-bone modifications (i.e., amide bond mimetics) well known to those skilled in the art. Such modifications include modifications of the amide nitrogen, the α-carbon, amide carbonyl, complete replacement of the amide bond, extensions, deletions or backbone crosslinks. Several peptide backbone modifications are known, including Ψ[CH₂S], TΨ8 CH₂NH], Ψ[CSNH₂], Ψ[NHCO], Ψ[COCH₂], and Ψ[(E) or (Z) CH═CH]. In the nomenclature used above, Ψ indicates the absence of an amidc bond. The structure that replaces the amide group is specified within the brackets. Other examples of isosteres include peptides substituted with one or more benzodiazepine molecules (see, e.g., James et al., Science 260, 1937-1942 (1993)).

Other possible modifications include an N-alkyl (or aryl) substitution (Ψ[CONR]), backbone crosslinking to construct lactams and other cyclic structures, substitution of all D-amino acids for all L-amino acids within the compound (“inverso” compounds) or retro-inverso amino acid incorporation (Ψ[NHCO]). By “inverso” is meant replacing L-amino acids of a sequence with D-amino acids, and by “retro-inverso” or “enantio-retro” is meant reversing the sequence of the amino acids (“retro”) and replacing the L-amino acids with D-amino acids. For example, if the parent peptide is Thr-Ala-Tyr, the retro modified form is Tyr-Ala-Thr, the inverso form is thr-ala-tyr, and the retro-inverso form is tyr-ala-thr (lower case letters refer to D-amino acids). Compared to the parent peptide, a retro-inverso peptide has a reversed backbone while retaining substantially the original spatial conformation of the side chains, resulting in a retro-inverso isomer with a topology that closely resembles the parent peptide. See Goodman et al., “Perspectives in Peptide Chemistry” pp. 283-294 (1981). See also U.S. Pat. No. 4,522,752 for further description of “retro-inverso” peptides.

Other derivatives of the modulator compounds of the invention include C-terminal hydroxymethyl derivatives, O-modified derivatives (e.g., C-terminal hydroxymethyl benzyl ether), N-terminally modified derivatives including substituted amides such as alkylamides and hydrazides and compounds in which a C-terminal phenylalanine residue is replaced with a phenethylamide analogue (e.g., Val-Phe-phenethylamide as an analogue of the tripeptide Val-Phe-Phe).

In a preferred example, the ACD of the modulator is modeled after the subregion of β-AP encompassing amino acid positions 17-20 (i.e., Leu-Val-Phe-Phe). Peptide subregions of Aβ₁₋₄₀ have been amino-terminally modified and evaluated for their ability to modulate aggregation of natural β-amyloid peptides. One subregion that was effective at inhibiting aggregation was Aβ₆₋₂₀ (i.e., amino acid residues 6-20 of the natural Aβ₁₋₄₀ peptide). Amino acid residues were serially deleted from the amino-terminus or carboxyl terminus of this subregion to further delineate a minimal subregion that was sufficient for aggregation inhibitory activity. This process defined Aβ₁₇₋₂₀ (Le., amino acid residues 17-20 of the natural Aβ₁₋₄₀ peptide) as a minimal subregion that, when appropriately modified, is sufficient for aggregation inhibitory activity. Accordingly, an “Aβ aggregation core domain” -within a modulator compound of the invention can be modeled after Aβ₁₇₋₂₀. In one example, the Aβ aggregation core domain comprises Aβ₁₇₋₂₀ itself (i.e., a peptide comprising the amino acid sequence leucine-valine-phenylalanine-phenylalanine). In other examples, the structure of Aβ₁₇₋₂₀ is used as a model to design an Aβ aggregation core domain having similar structure and function to Aβ₁₇₋₂₀. For example, peptidomimetics, derivatives or analogues of Aβ₁₇₋₂₀ (as described above) can be used as an Aβ aggregation core domain. In addition to Aβ₁₇₋₂₀, the natural Aβ peptide is likely to contain other minimal subregions that are sufficient for aggregation inhibitory activity.

One form of the β-amyloid modulator compound comprising an Aβ aggregation core domain modeled after Aβ₁₇₋₂₀ coupled directly or indirectly to at least one modifying group has the formula: 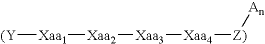
where Xaa₁ and Xaa₃ are amino acid structures; Xaa₂ is a valine structure; Xaa₄ is a phenylalanine structure; Y, which may or may not be present, is a peptidic structure having the formula (Xaa)ₐ, wherein Xaa is any amino acid structure and a is an integer from 1 to 15; Z, which may or may not be present, is a peptidic structure having the formula (Xaa)_{b}, wherein Xaa is any amino acid structure and b is an integer from 1 to 15; and A is a modifying group attached directly or indirectly to the compound and n is an integer, Xaa₁, Xaa₃, Y, Z, A and n being selected such that the compound modulates the aggregation or inhibits the neurotoxicity of natural β-amyloid peptides when contacted with the natural β-amyloid peptides.

Preferably, a modulator compound of the above formula inhibits aggregation of natural β-amyloid peptides when contacted with the natural β-amyloid peptides and/or inhibits Aβ neurotoxicity. Alternatively, the modulator compound can promote aggregation of natural Aβ-amyloid peptides when contacted with the natural β-amyloid peptides. The type and number of modifying groups (“A”) coupled to the modulator are selected such that the compound alters (and preferably inhibits) aggregation of natural β-amyloid peptides when contacted with the natural β-amyloid peptides. A single modifying group can be coupled to the modulator (i.e., n=1 in the above formula) or, alternatively, multiple modifying groups can be coupled to the modulator. In various embodiments, n is an integer between 1 and 60, between 1 and 30, between 1 and 10, between 1 and 5 or between 1 and 3.

Amino acid positions 18 (Val₁₈) and 20 (Phe₂₀) of Aβ₁₇₋₂₀ (corresponding to Xaa₂ and Xaa₄) are particularly important within the core domain for inhibitory activity of the modulator compound. Accordingly, these positions are conserved within the core domain in the formula shown above. The terms “valine structure” and “phenylalanine structure” as used in the above formula are intended to include the natural amino acids, as well as non-naturally-occurring analogues, derivatives and mimetics of valine and phenylalanine, respectively, (including D-amino acids) which maintain the functional activity of the compound. Moreover, although Val₁₈ and Phe₂₀ have an important functional role, it is possible that Xaa₂ and/or Xaa₄ can be substituted with other naturally-occurring amino acids that are structurally related to valine or phenylalanine, respectively, while still maintaining the activity of the compound. Thus, the terms “valine structure” is intended to include conservative amino acid substitutions that retain the activity of valine at Xaa₂, and the term “phenylalanine structure” is intended to include conservative amino acid substitutions that retain the activity of phenylalanine at Xaa₄. However, the term “valine structure” is not intended to include threonine.

In contrast to positions 18 and 20 of Aβ₁₇₋₂₀, a Phe to Ala substitution at position 19 (corresponding to Xaa₃) did not abolish the activity of the modulator, indicating position 19 may be more amenable to amino acid substitution. In various embodiments of the above formula, positions Xaa₁ and Xaa₃ are any amino acid structure. The term “amino acid structure” is intended to include natural and non-natural amino acids as well as analogues, derivatives and mimetics thereof, including D-amino acids. In a preferred example of the above formula, Xaa₁ is a leucine structure and Xaa₃ is a phenylalanine structure (i.e., modeled after Leu₁₇ and Phe₁₉, respectively, in the natural Aβ peptide sequence). The term “leucine structure” is used in the same manner as valine structure and phenylalanine structure described above. Alternatively, in another example, Xaa₃ is an alanine structure.

The four amino acid structure ACD of the modulator of the above formula can be flanked at the amino-terminal side, carboxy-terminal side, or both, by peptidic structures derived either from the natural Aβ peptide sequence or from non-Aβ sequences. The term “peptidic structure” is intended to include peptide analogues, derivatives and mimetics thereof, as described above. The peptidic structure is composed of one or more linked amino acid structures, the type and number of which in the above formula are variable. For example, in one example, no additional amino acid structures flank the Xaa₁-Xaa₂-Xaa₃-Xaa₄ core sequence (i.e., Y and Z are absent in the above formula). In another embodiment, one or more additional amino acid structures flank only the amino-terminus of the core sequences (i.e., Y is present but Z is absent in the above formula). In yet another embodiment, one or more additional amino acid structures flank only the carboxy-terminus of the core sequences (i.e., Z is present but Y is absent in the above formula). The length of flanking Z or Y sequences also is variable. For example, in one embodiment, a and b are integers from 1 to 15. More preferably, a and b are integers between 1 and 10. Even more preferably, a and b are integers between l and 5. Most preferably, a and b are integers between 1 and 3.

One form of the β-amyloid modulator compound comprising an Aβ aggregation core domain modeled after Aβ₁₇₋₂₀ coupled directly or indirectly to at least one modifying group has the formula:
A-(Y)-Xaa₁-Xaa₂-Xaa₃-Xaa₄-Z)-B
where Xaa₁ and Xaa₃ are amino acids or amino acid mimetics; Xaa₂ is valine or a valine mimetic; Xaa₄ is phenylalanine or a phenylalanine mimetic; Y, which may or may not be present, is a peptide or peptidomimetic having the formula (Xaa)ₐ, wherein Xaa is any amino acid or amino acid mimetic and a is an integer from 1 to 15; Z, which may or may not be present, is a peptide or peptidomimetic having the formula (Xaa)_{b}, wherein Xaa is any amino acid or amino acid mimetic and b is an integer from 1 to 15; and A and B, at least one of which is present, are modifying groups attached directly or indirectly to the amino terminus and carboxyl terminus, respectively, of the compound; Xaa₁, Xaa₃, Y, Z, A, and B being selected such that the compound modulates the aggregation or inhibits the neurotoxicity of natural β-amyloid peptides when contacted with the natural β-amyloid peptides.

In this embodiment, the modulator compound is specifically modified at either its amino-terminus, its carboxyl-terminus, or both. The terminology used in this formula is the same as described above. In one embodiment, the compound is modified only at its amino terminus (i.e., B is absent and the compound comprises the formula: A-(Y)-Xaa₁-Xaa₂-Xaa₃-Xaa₄-(Z)). In another embodiment, the compound is modified only at its carboxyl-terminus (i.e., A is absent and the compound comprises the formula: (Y)-Xaa₁-Xaa₂-Xaa₃-Xaa₄-Z)-B). In yet another embodiment, the compound is modified at both its amino- and carboxyl termini (i.e., the compound comprises the formula: A-(Y)-Xaa₁-Xaa₂-Xaa₃-Xaa₄-(Z)-B and both A and B are present). As described above, the type and number of amino acid structures which flank the Xaa₁-Xaa₂-Xaa₃-Xaa₄ core sequences in the above formula is variable. For example, in one example, a and b are integers from 1 to 15. More preferably, a and b are integers between 1 and 10. Even more preferably, a and b are integers between 1 and 5. Most preferably, a and b are integers between 1 and 3.

Preferred AP modulator compounds comprise modified forms of Aβ₁₄₋₂₁ (His-Gln-Lys-Leu-Val-Phe-Phe-Ala), or amino-terminal or carboxy-terminal deletions thereof, with a preferred “minimal core region” comprising Aβ₁₇₋₂₀. Accordingly, in specific embodiments, the invention provides compounds comprising the formula:
A-Xaa₁-Xaa₂-Xaa₃-Xaa₄Xaa₅-Xaa₆-Xaa₇-Xaa₈ -B
where Xaa₁ is a histidine structure; Xaa₂ is a glutamine structure; Xaa₃ is a lysine structure; Xaa₄ is a leucine structure; Xaa₅ is a valine structure; Xaa₆ is a phenylalanine structure; Xaa₇ is a phenylalanine structure; Xaa₈ is an alanine structure; A and B are modifying groups attached directly or indirectly to the amino terminus and carboxyl terminus, respectively, of the compound; and wherein Xaa₁-Xaa₂-Xaa₃, Xaa₁-Xaa₂, or Xaa₁ may or may not be present; Xaa₈ may or may not be present; and at least one of A and B is present.

In one specific example, the compound comprises the formula: A-Xaa₄-Xaa₅-Xaa₆-Xaa₇-B (eg., a modified form of Aβ₁₇₋₂₀, comprising an amino acid sequence Leu-Val-Phe-Phe).

In another specific example, the compound comprises the formula: A-Xaa₄-Xaa₅-Xaa₆-Xaa₇-Xaa₈-B (e.g., a modified form of Aβ_{17-21,} comprising an amino acid sequence Leu-Val-Phe-Phe-Ala).

In another specific example, the compound comprises the formula: A-Xaa₃-Xaa₄-Xaa₅-Xaa₆-Xaa₇-B (e.g., a modified form of Aβ₁₆₋₂₀, comprising an amino acid sequence Lys-Leu-Val-Phe-Phe).

In another specific example, the compound comprises the formula: A-Xaa₃-Xaa₄-Xaa₅-Xaa₆-Xaa₇-Xaa₈-B (e.g., a modified form of Aβ₁₆₋₂₁, comprising an amino acid sequence Lys-Leu-Val-Phe-Phe-Ala).

In another specific example, the compound comprises the formula: A-Xaa₂-Xaa₃-Xaa₄-Xaa₅-Xaa₆-Xaa₇-B (e.g., a modified form of Aβ₁₅₋₂₀, comprising an amino acid sequence Gln-Lys-Leu-Val-Phe-Phe).

In another specific example, the compound comprises the formula: A-Xaa₁-Xaa₃-Xaa₄-Xaa₃-Xaa₆-Xaa₇-Xaa₈-B (e.g., a modified form of Aβ₁₅₋₂₁, comprising an amino acid sequence Gln-Lys-Leu-Val-Phe-Phe-Ala).

In another specific embodiment, the compound comprises the formula: A-Xaa₁-Xaa₂-Xaa₃-Xaa₄-Xaa₅-Xaa₆-Xaa₇-B (e.g., a modified form of Aβ₁₄₋₂₀, comprising an amino acid sequence His-Gln-Lys-Leu-Val-Phe-Phe).

In another specific embodiment, the compound comprises the formula: A-Xaa₁-Xaa₂-Xaa₃-Xaa₄-Xaa₅-Xaa₆-Xaa₇-Xaa₈-B (e.g., a modified form of Aβ₁₄₋₂₁, comprising an amino acid sequence His-Gln-Lys-Leu-Val-Phe-Phe-Ala).

In preferred embodiments of the aforementioned specific embodiments, A or B is a cholanoyl structure or a biotin-containing structure.

In further experiments to delineate subregions of Aβ upon which an Aβ aggregation core domain can be modeled, it was shown that a modulator compound having inhibitory activity can comprise as few as three Aβ amino acids residues (e.g. Val-Phe-Phe, which corresponds to Aβ₁₈₋₂₀ or Phe-Phe-Ala, which corresponds to Aβ₁₉₋₂₁). The results also demonstrated that a modulator compound having a modulating group at its carboxy-terminus is effective at inhibiting Aβ aggregation. Still further, the results demonstrated that the cholyl group, as a modulating group, can be manipulated while maintaining the inhibitory activity of the compounds and that an iodotyrosyl can be substituted for phenylalanine (e.g., at position 19 or 20 of the Aβ sequence) while maintaining the ability of the compound to inhibit Aβ aggregation.

Still further, the results showed that compounds with inhibitory activity can be created using amino acids residues that are derived from the Aβ sequence in the region of about positions 17-21 but wherein the amino acid sequence is rearranged or has a substitution with a non-Aβ-derived amino acid. Examples of such compounds include PPI-426, in which the sequence of Aβ₁₇₋₂₁ (LVFFA) has been rearranged (FFVLA), PPI-372, in which-the sequence of Aβ₁₆₋₂₀ (KLVFF) has been rearranged (FKFVL), and PPI-388, -389 and -390, in which the sequence of Aβ₁₇₋₂₁ (LVFFA) has been substituted at position 17, 18, or 19, respectively, with an alanine residue (AVFFA) for PPI-388, LAFFA for PPI-389, and LVAFA for PPI-390. Another example of a peptide that can be used for this purpose is PPI-1019, which has the following sequence: N-methyl-D-Leu-D-Val-D-Phe-D-Phe-D-Leu)-NH₂ (U.S. Pat. No. 6,610,658).

The inhibitory activity of these compounds indicate that the presence in the compound of an amino acid sequence directly corresponding to a portion of Aβ is not essential for inhibitory activity, but rather suggests that maintenance of the hydrophobic nature of this core region, by inclusion of amino acid residues such as phenylalanine, valine, leucine, regardless of their precise order, can be sufficient for inhibition of Aβ aggregation. Accordingly, an Aβ aggregation core domain can be designed based on the direct Aβ amino acid sequence or can be designed based on a rearranged Aβ sequence which maintains the hydrophobicity of the Aβ subregion, e.g., the region around positions 17-20. This region of Aβ contains the amino acid residues Leu, Val, and Phe. Accordingly, preferred Aβ aggregation core domains are composed of at least three amino acid structures (as that term is defined hereinbefore, including amino acid derivatives, analogues and mimetics), wherein at least two of the amino acid structures are, independently, either a leucine structure, a valine structure or a phenylalanine structure (as those terms are defined hereinbefore, including derivatives, analogues and mimetics).

Thus, in another embodiment, the invention provides aβ-amyloid modulator compound comprising a formula: 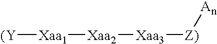
wherein Xaa₁, Xaa₂, and Xaa₃ are each amino acid structures and at least two of Xaa₁, Xaa₂, and Xaa₃ are, independently, selected from the group consisting of a leucine structure, a phenylalanine structure and a valine structure; Y, which may or may not be present, is a peptidic structure having the formula (Xaa)ₐ, wherein Xaa is any amino acid structure and a is an integer from 1 to 15; Z, which may or may not be present, is a peptidic structure having the formula (Xaa)_{b}, wherein Xaa is any amino acid structure and b is an integer from 1 to 15; and A is a modifying group attached directly or indirectly to the compound and n is an integer; Xaa₁, Xaa₂, Xaa₃, Y, Z, A, and n being selected such that the compound modulates the aggregation or inhibits the neurotoxicity of natural β-amyloid peptides when contacted with the natural β-amyloid peptides.

Preferably, the compound inhibits aggregation of natural β-amyloid peptides when contacted with the natural β-amyloid peptides. In preferred embodiments, Xaa₁ and Xaa₂ are each phenylalanine structures or Xaa₂ and Xaa₃ are each phenylalanine structures. “n” can be, for example, an integer between 1 and 5, whereas “a” and “b” can be, for example, integers between 1 and 5. The modifying group “A” preferably comprises a cyclic, heterocyclic or polycyclic group. More preferably, A contains a cis-decalin group, such as cholanoyl structure or a cholyl group. In other examples, A can comprise a biotin-containing group, a diethylene-triaminepentaacetyl group, a (−)-menthoxyacetyl group, a fluorescein-containing group or an N-acetylneuraminyl group. In yet other examples, the compound may promote aggregation of natural β-amyloid peptides when contacted with the natural β-amyloid peptides, may be further modified to alter a pharmacokinetic property of the compound or may be further modified to label the compound with a detectable substance.

In another embodiment, the invention provides a β-amyloid modulator compound comprising a formula: 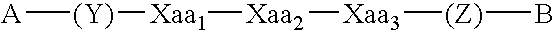
wherein Xaa₁, Xaa₂, and Xaa₃ are each amino acid structures and at least two of Xaa₁, Xaa₂, and Xaa₃ are, independently, selected from the group consisting of a leucine structure, a phenylalanine structure and a valine structure; Y, which may or may not be present, is a peptidic structure having the formula (Xaa)ₐ, wherein Xaa is any amino acid structure and a is an integer from 1 to 15; Z, which may or may not be present, is a peptidic structure having the formula (Xaa)_{b}, wherein Xaa is any amino acid structure and b is an integer from 1 to 15; and A and B, at least one of which is present, are modifying groups attached directly or indirectly to the amino terminus and carboxy terminus, respectively, of the compound; Xaa₁, Xaa₂, Xaa₃, Y, Z, A, and B being selected such that the compound modulates the aggregation or inhibits the neurotoxicity of natural β-amyloid peptides when contacted with the natural β-amyloid peptides.

Preferably, the compound inhibits aggregation of natural β-amyloid peptides when contacted with the natural β-amyloid peptides. In preferred embodiments, Xaa₁ and Xaa₂ are each phenylalanine structures or Xaa₂ and Xaa₃ are each phenylalanine structures. In one subembodiment, the compound comprises the formula:
A-(Y)-Xaa₁-Xaa₂-Xaa₃-(Z)

In another subembodiment, the compound comprises the formula:
(Y)-Xaa₁-Xaa₂-Xaa₃-(Z)-B
“n” can be, for example, an integer between 1 and 5, whereas “a” and “b” can be, for example, integers between 1 and 5. The modifying group “A” preferably comprises a cyclic, heterocyclic, or polycyclic group. More preferably, A contains a cis-decalin group, such as cholanoyl structure or a cholyl group. In other embodiments, A can comprise a biotin-containing group, a diethylene-triaminepentaacetyl group, a (−)menthoxyacetyl group, a fluorescein-containing group or an N-acetylneuraminyl group. In yet other embodiments, the compound may promote aggregation of natural β-amyloid peptides when contacted with the natural β-amyloid peptides, may be further modified to alter a pharmacokinetic property of the compound or may be further modified to label the compound with a detectable substance.

In preferred specific embodiments, the invention provides a β-amyloid modulator compound comprising a modifying group attached directly or indirectly to a peptidic structure, wherein the peptidic structure comprises amino acid structures having an amino acid sequence selected from the group consisting of His-Gln-Lys-Leu-Val-Phe-Phe-Ala, His-Gln-Lys-Leu-Val-Phe-Phe, Gln-Lys-Leu-Val-Phe-Phe-Ala, Gln-Lys-Leu-Val-Phe-Phe, Lys-Leu-Val-Phe-Phe-Ala, Lys-Leu-Val-Phe-Phe, Leu-Val-Phe-Phe-Ala, Leu-Val-Phe-Phe, Leu-Ala-Phe-Phe-Ala, Val-Phe-Phe, Phe-Phe-Ala, Phe-Phe-Val-Leu-Ala, Leu-Val-Phe-Phe-Lys, Leu-Val-Iodotyrosine-Phe-Ala, Val-Phe-Phe-Ala, Ala-Val-Phe-Phe-Ala, Leu-Val-Phe-Iodotyrosine-Ala, Leu-Val-Phe-Phe-Ala-Glu, Phe-Phe-Val-Leu, Phe-Lys-Phe-Val-Leu, Lys-Leu-Val-Ala-Phe, Lys-Leu-Val-Phe-Phe-βAla, and Leu-Val-Phe-Phe-DAla.

These specific compounds can be further modified to alter a pharmacokinetic property of the compound and/or further modified to label the compound with a detectable substance.

Within a modulator compound of the invention, a peptidic structure (such as an Aβ derived peptide, or an Aβ aggregation core domain, or an amino acid sequence corresponding to a rearranged Aβ aggregation core domain) is coupled directly or indirectly to at least one modifying group (abbreviated as MG). In one embodiment, a modulator compound of the invention comprising an aggregation core domain coupled to a modifying group, the compound can be illustrated schematically as MG-ACD. The term “modifying group” is intended to include structures that are directly attached to the peptidic structure (e.g., by covalent coupling), as well as those that are indirectly attached to the peptidic structure (e.g. by a stable non-covalent association or by covalent coupling to additional amino acid residues, or mimetics, analogues or derivatives thereof, which may flank the Aβ-derived peptidic structure). For example, the modifying group can be coupled to the amino-terminus or Carboxyl-terminus of an Aβ-derived peptidic structure, or to a peptidic or peptidomimetic region flanking the core domain. Alternatively, the modifying group can be coupled to a side chain of at least one amino acid residue of an Aβ-derived peptidic structure, or to a peptidic or peptidomimetic region flanking the core domain (e.g., through the epsilon amino group of a lysyl residue(s), through the carboxyl group of an aspartic acid residue(s) or a glutamic acid residue(s), through a hydroxy group of a tyrosyl residue(s), a serine residue(s) or a threonine residue(s) or other suitable reactive group on an amino acid side chain). Modifying groups covalently coupled to the peptidic structure can be attached by means and using methods well known in the art for linking chemical structures, including, for example, amide, alkylamino, carbamate, or urea bonds.

The term “modifying group” is intended to include groups that are not naturally coupled to natural Aβ peptides in their native form. Accordingly, the term “modifying group” is not intended to include hydrogen. The modifying group(s) is selected such that the modulator compound alters, and preferably inhibits, aggregation of natural β-amyloid peptides when contacted with the natural β-amyloid peptides or inhibits the neurotoxicity of natural β-amyloid peptides when contacted with the natural β-amyloid peptides. Although not intending to be limited by mechanism, the modifying group(s) of the modulator compounds of the invention is thought to function as a key pharmacophore that is important for conferring on the modulator the ability to disrupt Aβ polymerization.

In a preferred embodiment, the modifying group(s) comprises a cyclic, heterocyclic or polycyclic group. The term “cyclic group,” as used herein, is intended to include cyclic saturated or unsaturated (i.e., aromatic) group having from about 3 to 10, preferably about 4 to 8, and more preferably about 5 to 7, carbon atoms. Exemplary cyclic groups include cyclopropyl, cyclobutyl, cyclopentyl, cyclohexyl, and cyclooctyl. Cyclic groups may be unsubstituted or substituted at one or more ring positions. Thus, a cyclic group may be substituted with, e.g., halogens, alkyls, cycloalkyls, alkenyls, alkynyls, aryls, heterocycles, hydroxyls, aminos, nitros, thiols amines, imines, amides, phosphonates, phosphines, carbonyls, carboxyls, silyls, ethers, thioethers, sulfonyls, sulfonates, selenoethers, ketones, aldehydes, esters, —CF₃, —CN, or the like.

The term “heterocyclic group” is intended to include cyclic saturated or unsaturated (i.e., aromatic) group having from about 3 to 10, preferably about 4 to 8, and more preferably about 5 to 7, carbon atoms, wherein the ring structure includes about one to four heteroatoms. Heterocyclic groups include pyrrolidine, oxolane, thiolane, imidazole, oxazole, piperidine, piperazine, morpholine. The heterocyclic ring can be substituted at one or more positions with such substituents as, for example, halogens, alkyls, cycloalkyls, alkenyls, alkynyls, aryls, other heterocycles, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, —CN, or the like. Heterocycles may also be bridged or fused to other cyclic groups.

The term “polycyclic group” as used herein is intended to refer to two or more saturated or unsaturated (i.e., aromatic) cyclic rings in which two or more carbons are common to two adjoining rings, e.g., the rings are “fused rings.” Rings that are joined through non-adjacent atoms are termed “bridged” rings. Each of the rings of the polycyclic group can be substituted with such substituents as described above, as for example, halogens, alkyls, cycloalkyls, alkenyls, alkynyls, hydroxyl, amino, nitro, thiol, amines, imines, amides, phosphonates, phosphines, carbonyls, carboxyls, silyls, ethers, thioethers, sulfonyls, selenoethers, ketones, aldehydes, esters, —CF₃, —CN, or the like.

A preferred polycyclic group is a group containing a cis-decalin structure. Although not intending to be limited by mechanism, it is thought that the “bent” conformation conferred on a modifying group by the presence of a cis-decalin structure contributes to the efficacy of the modifying group in disrupting Aβ polymerization. Accordingly,.other structures that mimic the “bent” configuration of the cis-decalin structure can also be used as modifying groups. An example of a cis-decalin containing structure that can be used as a modifying group is a cholanoyl structure, such as a cholyl group. For example, a modulator compound can be modified at its amino terminus with a cholyl group by reacting the aggregation core domain with cholic acid, a bile acid. Moreover, a modulator compound can be modified at its carboxy terminus with a cholyl group according to methods known in the art (see, e.g., Wess et al., Tetrahedron Letters 34, 817-822 (1993); Wess et al., Tetrahedron Letters 33, 195-198 (1992); and Kramer et al., J. Biol. Chem. 267, 18598-18604 (1992)). Cholyl derivatives and analogues can also be used as modifying groups. For example, a preferred cholyl derivative is Aic (3-O-aminoethyl-iso)-cholyl), which has a free amino group that can be used to further modify the. modulator compound (e.g., a chelation group for ^{99m}Tc can be introduced through the free amino group of Aic). As used herein, the term “cholanoyl structure” is intended to include the cholyl group and derivatives and analogues thereof, in particular those that retain a four-ring cis-decalin configuration. Examples of cholanoyl structures include groups derived from other bile acids, such as deoxycholic acid, lithocholic acid, ursodeoxycholic acid, chenodeoxycholic acid and hyodeoxycholic acid, as well as other related structures such as cholanic acid, bufalin and resibufogenin (although the latter two compounds are not preferred for use as a modifying group). Another example of a cis-decalin containing compound is 5β-cholestan-3α-ol (the cis-decalin isomer of (+)-dihydrocholesterol). For further description of bile acid and steroid structure and nomenclature, see Nes et al., Biochemistry of Steroids and Other Isopentanoids, University Park Press, Baltimore, Md., Chapter 2.

In addition to cis-decalin containing groups, other polycyclic groups may be used as modifying groups. For example, modifying groups derived from steroids or β-lactams may be suitable modifying groups. In one example, the modifying group is a “biotinyl structure,” which includes biotinyl groups and analogues and derivatives thereof (such as a 2-iminobiotinyl group). In another example, the modifying group can comprise a “fluorescein-containing group,” such as a group derived from reacting an Aβ-derived peptidic structure with 5-(and 6-)-carboxyfluorescein, succinimidyl ester or fluorescein isothiocyanate. In various other embodiments, the modifying group(s) can comprise an N-acetylneuraminyl group, a trans4-cotininecarboxyl group, a 2-imino-1-imidazolidineacetyl group, an (S)-(−)-indoline-2-carboxyl group, a (−)-menthoxyacetyl group, a 2-norbornaneacetyl group, a γ-oxo-5-acenaphthenebutyryl, a (−)-2-oxo-4thiazolidinecarboxyl group, a tetrabydro-3-furoyl group, a 2-iminobiotinyl group, a diethylenetriaminepentaacetyl group, a 4-morpholinecarbonyl group, a 2-thiopheneacetyl group, or a 2-thiophenesulfonyl group.

Preferred modifying groups include groups comprising cholyl structures, biotinyl structures, fluorescein-containing groups, a diethylene-triaminepentaacetyl group, a (−)-menthoxyacetyl group, and an N-acetylneuraminyl group. More preferred modifying groups those comprising a cholyl structure, or an iminiobiotinyl group.

In addition to the cyclic, heterocyclic and polycyclic groups discussed above, other types of modifying groups can be used in a modulator of the invention. For example, small hydrophobic groups may be suitable modifying groups. An example of a suitable-non-cyclic modifying group is an acetyl group.

Yet another type of modifying group is a compound that contains a non-natural amino acid that acts as a beta-turn mimetic, such as a dibenzofuran-based amino acid described in Tsang et al., J. Am. Chem. Soc. 116, 3988-4005 (1994); Diaz et al., Tetrahedron Letters 41, 5725-5728 (1991); and Diaz et al., J. Chem. Soc. 114, 8316-8318 (1992). An example of such a modifying group is a peptide-aminoethyldibenzofuranyl-proprionic acid (Adp) group (e.g., DDIIL-Adp). This type of modifying group further can comprise one or more N-methyl peptide bonds to introduce additional steric hindrance to the aggregation of natural β-AP when compounds of this type interact with natural β-AP.

Aβ-amyloid modulator compounds used in the invention can be further modified to alter the specific properties of the compound while retaining the ability of the compound to alter Aβ aggregation and inhibit Aβ neurotoxicity. For example, in one example, the compound is further modified to alter a pharmacokinetic property of the compound, such as in vivo stability or half-life. In another example, the compound is further modified to label the compound with a detectable substance. In yet another embodiment, the compound is further modified to couple the compound to an additional therapeutic moiety. Schematically, a modulator of the invention comprising an Aβ aggregation core domain coupled directly or indirectly to at least one modifying group can be illustrated as MG-ACD, whereas this compound which has been further modified to alter the properties of the modulator can be illustrated as MG-ACD-CM, wherein CM represents an additional chemical modification.

To further chemically modify the compound, such as to alter the pharmacokinetic properties of the compound, reactive groups can be derivatized. For example, when the modifying group is attached to the amino-terminal end of the aggregation core domain, the carboxy-terminal end of the compound can be further modified. Preferred C-terminal modifications include those that reduce the ability of the compound to act as a substrate for carboxypeptidases. Examples of preferred C-terminal modifiers include an amide group, an ethylamide group and various non-natural amino acids, such as D-amino acids and β-alanine. Alternatively, when the modifying group is attached to the carboxyl-terminal end of the aggregation core domain, the amino-terminal end of the compound can be further modified, for example, to reduce the ability of the compound to act as a substrate for aminopeptidases.

A modulator compound can be further modified to label the compound by reacting the compound with a detectable substance. Suitable detectable substances include various enzymes, prosthetic groups, fluorescent materials, luminescent materials and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; and examples of suitable radioactive material include ¹⁴C, ¹²³I, ¹²⁴I, ¹²⁵I, ¹³¹I, ^{99m}Tc, ³⁵S, or ³H. In a preferred embodiment, a modulator compound is radioactively labeled with ¹⁴C, either by incorporation of ¹⁴C into the modifying group or one or more amino acid structures in the modulator compound. Labeled modulator compounds can be used to assess the in vivo pharmacokinetics of the compounds, as well as to detect Aβ aggregation, for example for diagnostic purposes. Aβ aggregation can be detected using a labeled modulator compound either in vivo or in an in vitro sample derived from a subject.

Preferably, for use as an in vivo diagnostic agent, a modulator compound of the invention is labeled with radioactive technetium or iodine. Accordingly, in one embodiment, the invention provides a modulator compound labeled with technetium, preferably ^{99m}Tc. Methods for labeling peptide compounds with technetium are known in the art (see, e.g. U.S. Pat. Nos. 5,443,815; 5,225,180; and 5,405,597; Stepniak-Biniakiewicz et al., J. Med. Chem. 35, 274-279 (1992); Fritzberg et al., Proc. Natl. Acad. Sci U.S.A. 85, 4025-4029 (1988); Baidoo et al., Cancer Res. Suppl. 50, 799s-803s (1990); and Regan et al., Science 270, 980-982 (1995)). A modifying group can be chosen that provides a site at which a chelation group for ^{99m}Tc can be introduced, such as the Aic derivative of cholic acid, which has a free amino group. In another example, the a modulator compound labeled with radioactive iodine can be used. For example, a phenylalanine residue within the Aβ sequence (such as Phe₁₉ or Phe₂₀) can be substituted with radioactive iodotyrosyl. Any of the various isotopes of radioactive iodine can be incorporated to create a diagnostic agent. Preferably, ¹²³I (half-life=13.2 hours) is used for whole body scintigraphy, ¹²⁴I (half life=4 days) is used for positron emission tomography (PET), ¹²⁵I (half life=60 days) is used for metabolic turnover studies and ¹³¹I (half life=8 days) is used for whole body counting and delayed low resolution imaging studies.

Furthermore, an additional modification of a modulator compound used in the invention can serve to confer an additional therapeutic property on the compound. That is, the additional chemical modification can comprise an additional functional moiety. For example, a functional moiety which serves to break down or dissolve amyloid plaques can be coupled to the modulator compound. In this form, the MG-ACD portion of the modulator serves to target the compound to Aβ peptides and disrupt the polymerization of the Aβ peptides, whereas the additional functional moiety serves to break down or dissolve amyloid plaques after the compound has been targeted to these sites.

In an alternative chemical modification, a β-amyloid compound of the invention is prepared in a “prodrug” form, wherein the compound itself does not modulate Aβ aggregation, but rather is capable of being transformed, upon metabolism in vivo, into a β-amyloid modulator compound as defined herein. For example, in this type of compound, the modulating group can be present in a prodrug form that is capable of being converted upon metabolism into the form of an active modulating group. Such a prodrug form of a modifying group is referred to herein as a “secondary modifying group.” A variety of strategies are known in the art for preparing peptide prodrugs that limit metabolism in order to optimize delivery of the active form of the peptide-based drug (see, e.g., Moss, (1995) in Peptide-Based Drug Design: Controlling Transport and Metabolism, Taylor, M. D. and Amidon, G. L. (eds), Chapter 18. Additionally strategies have been specifically tailored to achieving CNS delivery based on “sequential metabolism” (see, e.g., Bodor et al., Science 257, 1698-1700 (1992); Prokai et al., J. Am. Chem. Soc. 116, 2643-2644 (1994); Bodor et al., (1995) in Peptide-Based Drug Design: Controlling Transport and Metabolism, Taylor, M. D. and Amidon, G. L. (eds), Chapter 14. In one example of a prodrug form of a modulator of the invention, the modifying group comprises an alkyl ester to facilitate blood-brain barrier permeability.

Modulator compounds of the invention can be prepared by standard techniques that are known in the art. The peptide component of a modulator composed, at least in part, of a peptide, can be synthesized using standard techniques such as those described in Bodansky, M. Principles of Peptide Synthesis, Springer Verlag, Berlin (1993) and Grant, (ed.). Synthetic Peptides: A User's Guide, W.H. Freeman and Company, New York (1992). Automated peptide synthesizers are commercially available (e.g., Advanced ChemTech Model 396; Milligen/Biosearch 9600). Additionally, one or more modulating groups can be attached to the Aβ-derived-peptidic component (eg., an Aβ aggregation core domain) by standard methods, for example using methods for reaction through an amino group (e.g., the alpha-amino group at the amino-terminus of a peptide), a carboxyl group (e.g., at the carboxy terminus of a peptide), a hydroxyl group (e.g., on a tyrosine, scrine or threonine residue) or other suitable reactive group on an amino acid side chain (see, e.g., Greene et al., Protective Groups in Organic Synthesis, John Wiley and Sons, Inc., New York (1991)).

Additional examples of peptides that can be used in the present invention as inhibitors of Aβ polymerization are those described in WO 97/21728 and U.S. Pat. No. 6,331,440 B1, which are incorporated herein by reference. These peptides bind to the Lys-Leu-Val-Phe-Phe-sequence in Aβ and can be defined by the formula (I):
R₁-A′-Y′-Leu-X′-Z′-B′—R₂ (I)
in which

X′ means any group or amino acid imparting to the compound of formula (I) the ability to bind to the KLVFF-sequence in Aβ or two amino acids imparting the same ability;

Y′ means any amino acid;

Z′ means any non-acidic amino acid;

A′ means a direct bond or an ax-amino acid bonded at the carboxyl terminal of the α-carboxy group or a di-, tri-, tetra- or pentapeptide bonded at the carboxyl terminal of the α-carboxy group;

B′ means a direct bond or an α-amino acid bonded at the a-nitrogen or a di-, tri-, tetra- or pentapeptide bonded at the 0-nitrogen or the N-terminal α-amino acid;

R₁ is H or —CO—R₃ bonded at the α-amino group of A′;

R₂ is H, —OR₄ or NR₅R₆, all bonded to the α-carboxyl group of the α-carboxyterminal of B′;

R₃ is a straight or branched carbon chain of 1-4 carbon atoms;

R₄ is a straight or branched carbon chain of 1-4 carbon atoms;

R₅ and R.sub_{.6} independently are H, alkyl, cycloalkyl, aryl or substituted aryl or together are —(CH₂)ₙ—, where n is 4-5;

R₁ and R₂ together can form a hydrocarbon ring or heterocyclic ring; and

all the α-amino acids can be wither D- or L-isomers.

By alkyl is preferably meant a chain of 4 or less carbon atoms, e.g., methyl; ethyl, propyl or butyl; by cycloalkyl is preferably meant a ring of 3, 4, 5, or 6 carbon atoms; aryl preferably means a phenyl group, which can be substituted, preferably by a methyl, ethyl, propyl or butyl group, an amino or a methoxy, ethoxy, propoxy, or butoxy group.

As with the other peptides described herein, peptides within the formula noted above can include one or more D amino acids, e.g., the peptides can include all D amino acids.

In one example of a compound of the formula set forth above, Y′ is Lys, and in another example, Z′ is Phe, resulting in a compound of the following formula:

R₁—A′-NH—CH[—(CH₂)₄—NH₂]—CO-Leu-X′-NH—CH[—CH₂-Ph]-CO—B′—R₂.

In another examples, Y′ is Phe; X′ is Val-Val; and/or R₁ is acetyl. In a further example, R₁ is H, while in yet another example, R₂ is H. Alternatively, R₁ and R₂ can both be H.

Specific examples of peptides described in U.S. Pat. No. 6,331,440 that can be used in the present invention are as follows: AcQKLVFFNH₂. Additional examples of peptides that can be used are those comprising the following sequences, with or without one or more terminal modifications: HHQKLVFFAE, GYEVHHQKLV, YEVHHQKLVF, VHHQKLVFFA, EVHHQKLVFF, VHHQKLVFF, HHQKLVFF, HQKLVFF, HHQKLVF, QKLVFF, HQKLVF, KLVFF, LVFF, and KLVF.

Additional peptides that can be used as antifibrillogenic agents in the methods and compositions of the present invention include those described in U.S. Pat. Nos. 6,462,171 B1 and 5,948,73, the teachings of both of which are incorporated by reference herein. These peptides are specifically designed to interfere with the β-sheet conformation of proteins or peptides involved in the formation of amyloid deposits. In particular, the peptides include a hydrophobic segment of at least three amino acid residues that is interrupted by one or more β-sheet blocking amino acid residues (e.g., Pro, Gly, Asn, or His) that do not substantially change the hydrophobicity of the segment. Preferably, the hydrophobic segment is homologous to a sequence of the β-sheet forming region of the protein against which antifibrillogenic activity is desired (e.g., in the case of Aβ, KLVFFAED). Examples of these peptides include the following: Ser-Arg-Gly-Asp-Leu-Pro-Phe-Phe-Pro-Val-Pro-Ile-Gly-Asp-Ser, Arg-Asp-Leu-Pro-Phe-Phe-Pro-Val-Pro-Ile-Asp; Arg-Asp-Phe-Ile-Pro-Leu-Pro-Leu-Asp; Arg-Asp-Tyr-Leu-Pro-Tyr-Tyr-Pro-Leu-Asp; Arg-Asp-Leu-Pro-Phe-Phe-Pro-Val-Pro-Ile-Asp; Arg-Asp-Leu-Pro-Phe-Phe-Pro-Val-Asp; Leu-Pro-Phe-Phe-Pro-Val-Asp; Leu-Pro-Phe-Phe-Val-Asp; Leu-Pro-Phe-Phe-Asp; Leu-Pro-Phe-Phe; Leu-Val-Pro-Phe-Asp; Leu-Phe-Pro-Phe-Asp; Pro-Leu-Phe-Phe-Asp; Leu-Val-Phe-Pro-Asp; Lys-Leu-Pro-Phe-Phe; Lys-Leu-Val-Pro-Phe; Lys-Pro-Val-Phe-Phe; Val-His-Val-Ser-Glu-Glu-Gly-Thr-Glu-Pro-Ala; Arg-Asp-Leu-Pro-Ile-Val,-Pro-Leu-Pro-De-Asp; Leu-Pro-Ile-Val-Pro-Leu-Asp; and Leu-Pro-Ile-Val-Asp. Additional peptides of this type can be identified using the methods described in, for example, U.S. Pat. Nos. 5,948,763 and 6,462,171 B1, and then used in the methods and compositions of the present invention.

As is discussed above, agents in addition to an akylsulfonic acid, as described herein, and a peptide, peptidomimetic compound, or antibody can be used in combination with these agents in the present invention. Several examples of such additional agents are provided above. Description of yet another type of an additional agent that can be used in the invention is as follows.

Other compounds that can be used in the invention include, for example, compounds of Formula (I-B): 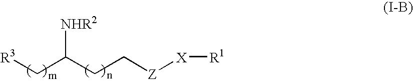
wherein:

X is oxygen or nitrogen;

Z is C═O, S(O)₂, or P(O)OR⁷;

m and n are each independently 0, 1, 2, 3, 4, 5, 6, 7, 8, 9 or 10;

R¹ and R⁷ are each independently hydrogen, metal ion, alkyl, mercaptoalkyl, alkenyl, alkynyl, cycloalkyl, aryl, a moiety together with X to form natural or unnatural amino acid residue, or —(CH₂)ₚ—Y;

Y is hydrogen or a heterocyclic moiety selected from the group consisting of thiazolyl, triazolyl, tetrazolyl, amidino, imidazolyl, benzothiazolyl, and benzoimidazolyl;

p is 0, 1, 2, 3, or 4;

R² is hydrogen, alkyl, mercaptoalkyl, alkenyl, alkynyl, cycloalkyl, aryl, alkylcaebonyl, arylcarbonyl, or alkoxycarbonyl;

R³ is hydrogen, amino, cyano, alkyl, mercaptoalkyl, alkenyl, alkynyl, cycloalkyl, heterocyclic, substituted or unsubstituted aryl, heteroaryl, thiazolyl, triazolyl, tetrazolyl, amidino, imidazolyl, benzothiazolyl, or benzoimidazolyl, and pharmaceutically acceptable salts, esters, and prodrugs thereof.

In a further embodiment, m is 0, 1, or 2. In another further embodiment, n is 0, 1, or 2. In another further embodiment, R³ is aryl, e.g., heteroaryl or phenyl. In yet another embodiment, Z is S(O)₂.

In another embodiment, the compound of the invention is of the Formula (II-B) 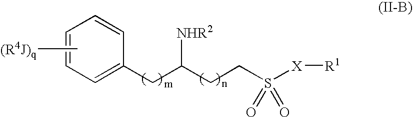
wherein:

each R⁴ is independently selected from the group consisting of hydrogen, halogen, hydroxyl, thiol, amino, amidino, cyano, nitro, alkyl, aryl, carbocyclic or heterocyclic;

J is absent, oxygen, nitrogen, sulfur, or a divalent link-moiety comsisting of, without limiting to, lower alkylene, alkylenyloxy, alkylenylamino, alkylenylthio, alkylenyloxyalkyl, alkylenylamonialkyl, alkylenylthioalkyl, alkenyl, alkenyloxy, alkenylamino, or alkenylthio; and

q is 1, 2, 3, 4, or 5, and pharmaceutically acceptable salts, esters and prodrugs thereof.

In a further embodiment, R⁴ is aryl, e.g., substituted or unsubstituted phenyl. In another embodiment, R⁴ is halogen (e.g., chlorine, fluorine, bromine, or iodine). In yet another embodiment, R⁴ is alkyl, e.g., methyl, ethyl, propyl, butyl, pentyl, trifluoromethyl, etc. In another embodiment, J is absent or oxygen. In a further embodiment, m is 1 or n is 1. In another further embodiment, the compound can be R- or S-isomer.

In a further embodiment, the compound is selected from the group consisting of:

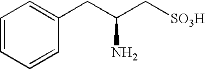

(S)-2-amino-3-phenylpropane-
1-sulfonic acid

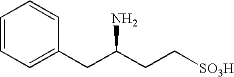

(R)-3-amino-4-phenylbutane-
1-sulfonic acid

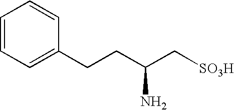

(S)-2-amino-4-phenylbutane-
1-sulfonic acid

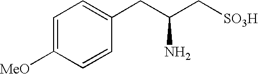

(S)-2-amino-
3-(4-methoxyphenyl)-propane-
1-sulfonic acid

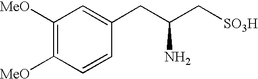

(S)-2-amino-
3-(3,4-dimethoxhenyl)-
propane-1-sulfonic acid

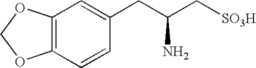

(S)-2-amino-
3-benzo[1,3]dioxol-5-yl-propane-
1-sulfonic acid

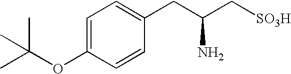

(S)-2-amino-
3-(4-tert-butoxyphenyl)-
propane-1-sulfonic acid

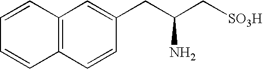

(S)-2-amino-
3-naphthalen-2-yl-propane-
1-sulfonic acid

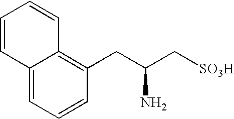

(S)-2-amino-3-naphthalen-1-yl-
propane-1-sulfonic acid

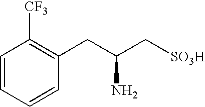

(S)-2-amino-
3-(2-trifluoromethylphenyl)-
propane-1-sulfonic acid

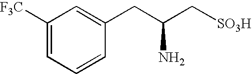

(S)-2-amino-
3-(3-trifluoromethylphenyl)-
propane-1-sulfonic acid

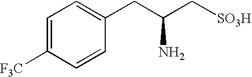

(S)-2-amino-
3-(4-trifluoronietbylphenyl)-
propane-1-sulfonic acid

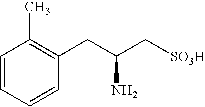

(S)-2-amino-
3-(2-methylphenyl)-propane-
1-sulfonic acid

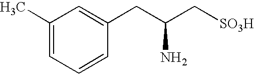

(S)-2-amino-
3-(3-methylphenyl)-propane-
1-sulfonic acid

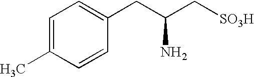

(S)-2-amino-3-(4-methylphenyl)-
propane-1-sulfonic acid

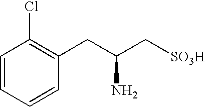

(S)-2-amino-
3-(2-chlorophenyl)-propane-
1-sulfonic acid

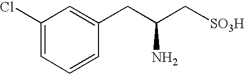

(S)-2-amino-
3-(3-chlorophenyl)-propane-
1-sulfonic acid

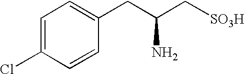

(S)-2-amino-3-(4-chlorophenyl)-
propane-1-sulfonic acid

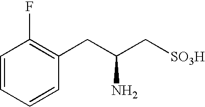

(S)-2-amino-3-(2-fluorophenyl)-
propane-1-sulfonic acid

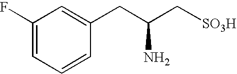

(S)-2-amino-
3-(3-fluorophenyl)-propane-
1-sulfonic acid

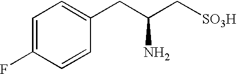

(S)-2-amino-3-(4-fluorophenyl)-
propane-1-sulfonic acid

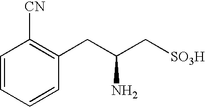

(S-2-amino-3-(2-cyanophenyl)-
propane-1-sulfonic acid

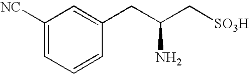

(S)-2-amino-
3-(3-cyanophenyl)-propane-
1-sulfonic acid

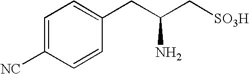

(S)-2-amino-3-(4-cyanophenyl)-
propane-1-sulfonic acid

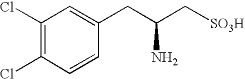

(S)-2-amino-
3-(3,4-dichlorophenyl)-propane-
1-sulfonic acid

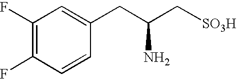

(S)-2-amino-
3-(3,4-difluorophenyl)-
propane-1-sulfonic acid

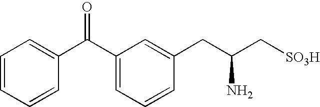

(S)-2-amino-3-(3-benzoylphenyl)-
propane-1-sulfonic acid

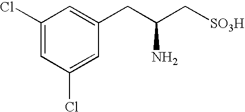

(S)-2-amino-
3-(3,5-dichlorophenyl)-propane-
1-sulfonic acid

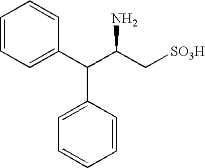

(R)-2-amino-
3,3-diphenylpropane-
1-sulfonic acid

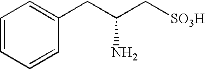

(R)-2-amino-3-phenylpropane-
1-sulfonic acid

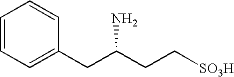

(S)-3-amino-4-phenylbutane-
1-sulfonic acid

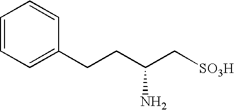

(R)-2-amino-4-phenylbutane-
1-sulfonic acid

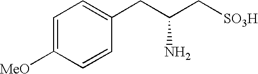

(R)-2-amino-
3-(4-methoxyphenyl)-propane-
1-sulfonic acid

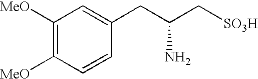

(R)-2-amino-
3-(3,4-dimethoxyphenyl)-
propane-1-sulfonic acid

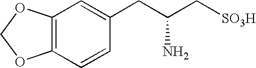

(R)-2-amino-
3-benzo[1,3]dioxol-5-yl-propane-
1-sulfonic acid

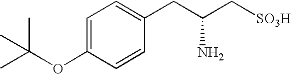

(R)-2-amino-
3-(4-tert-butoxyphenyl)-
propane-1-sulfonic acid

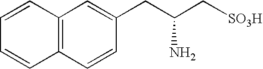

(R)-2-amino-
3-naphthalen-2-yl-propane-
1-sulfonic acid

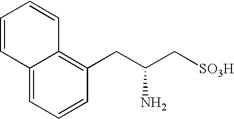

(R)-2-amino-3-naphthalen-1-yl-
propane-1-sulfonic acid

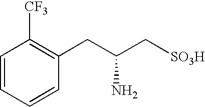

(R)-2-amino-
3-(2-trifluoromethylphenyl)-
propane-1-sulfonic acid

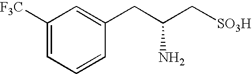

(R)-2-amino-
3-(3-trifluoromethylphenyl)-
propane-1-sulfonic acid

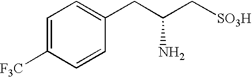

(R)-2-amino
3-(4-trifluoromethylphenyl)-
propane-1-sulfonic acid

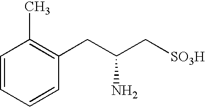

(R)-2-amino-
3-(3-methylphenyl)-propane-
1-sulfonic acid

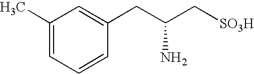

(R)-2-amino-
3-(3-methylphenyl)-propane-
1-sulfonic acid

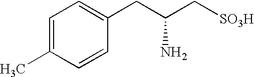

(R)-2-amino-3-(4-methylphenyl)-
propane-1-sulfonic acid

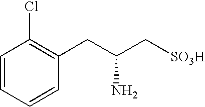

(R)-2-amino-
3-(2-chlorophenyl)-propane-
1-sulfonic acid

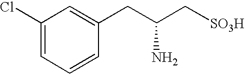

(R)-2-amino-
3-(3-chlorophenylpropane-
1-sulfonic acid

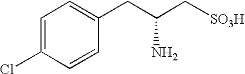

(R)-2-amino-3-(4-chlorophenyl)-
propane-1-sulfonic acid

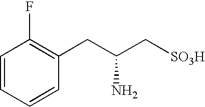

(R)-2-amino-
3-(2-fluorophenyl)-propane-
1-sulfonic acid

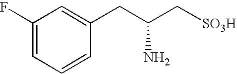

(R)-2-amino-
3-(3-fluorophenyl)-propane-
1-sulfonic acid

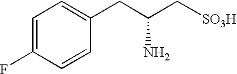

(R)-2-amino-3-(4-fluorophenyl)-
propane 1-sulfonic acid

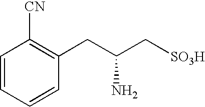

(R)-2-amino-3-(2-cyanophenyl)-
propane-1-sulfonic acid

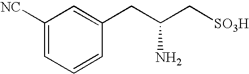

(R)-2-amino-
3-(3-cyanophenyl)-propane-
1-sulfonic acid

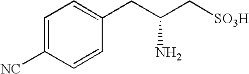

R-2-amino-3-(4-cyanophenyl)-
propane-1-sulfonic acid

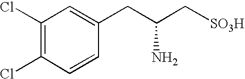

(R)-2-amino-
3-(3,4-dichlorophenyl)-propane-
1-sulfonic acid

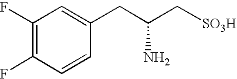

(R)-2-amino-
3-(3,4-difluorophenyl)-
propane-1-sulfonic acid

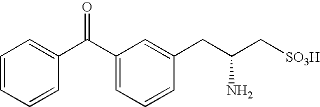

(R)-2-amino-3-(3-benzoylpbenyl)-
propane-1-sulfonic acid

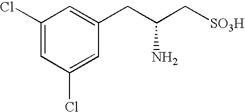

(R)-2-amino-
3-(3,5-dichlorophenyl)-propane-
1-sulfonic acid

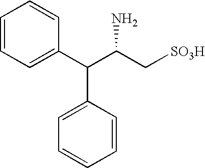

(S)-2-amino-
3,3-diphenylpropane-
1-sulfonic acid

and pharmaceutically acceptable salts, prodrugs, and esters thereof.

In a further embodiment, the compound is selected from the group consisting of: 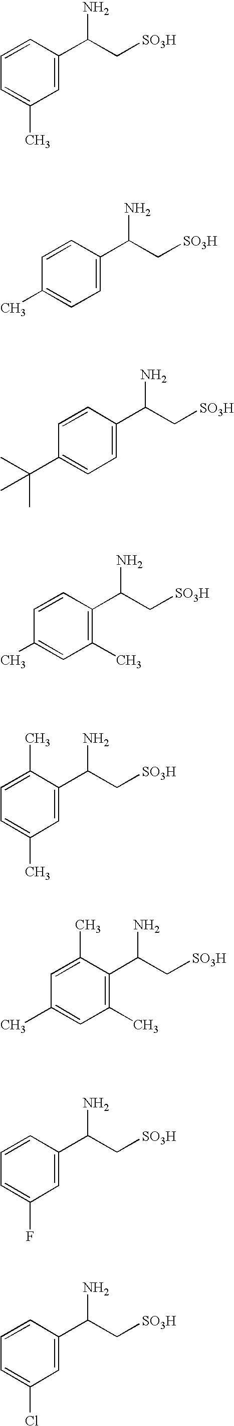 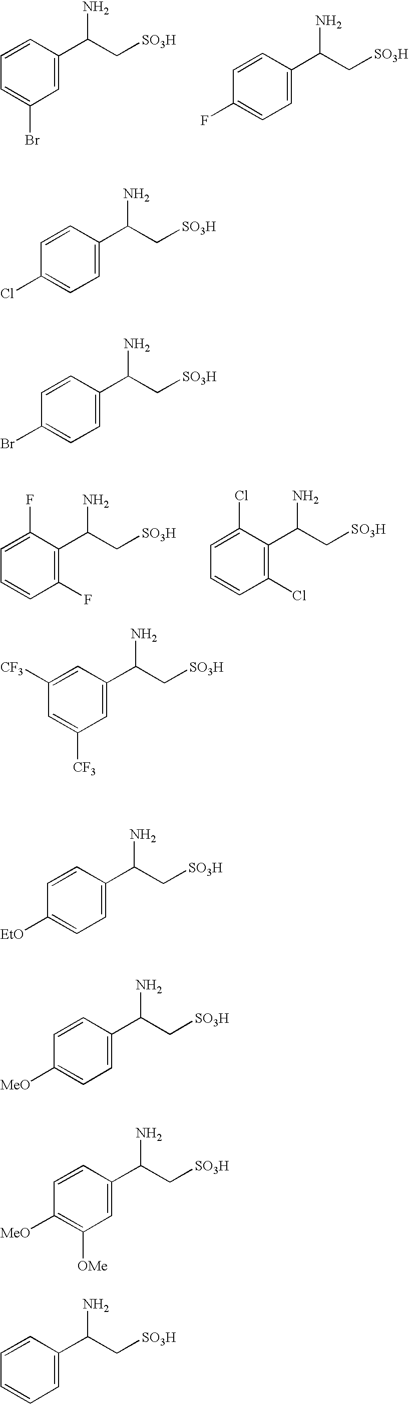 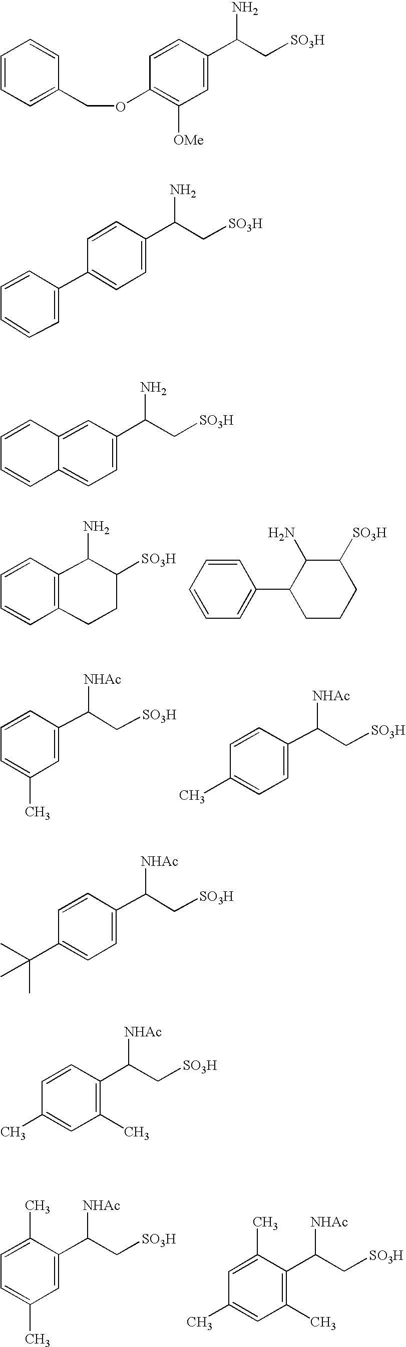 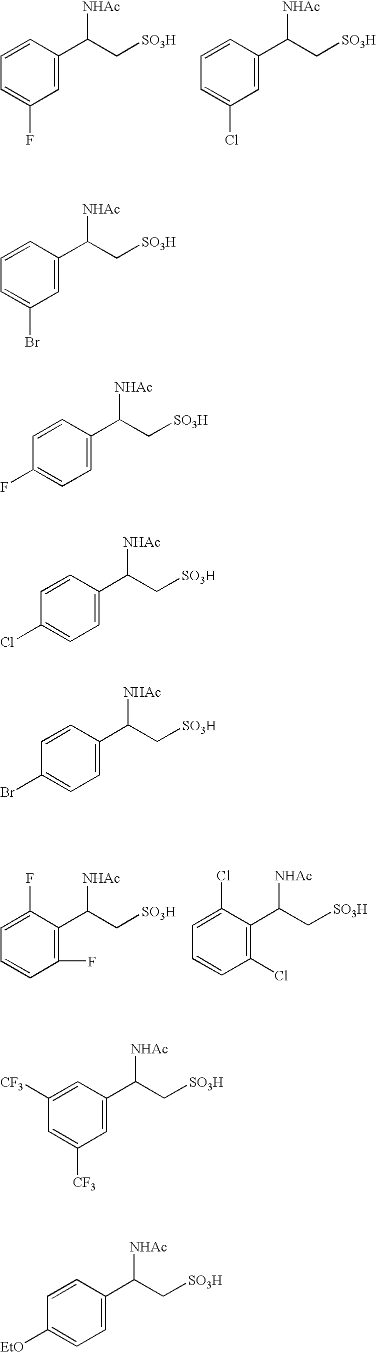 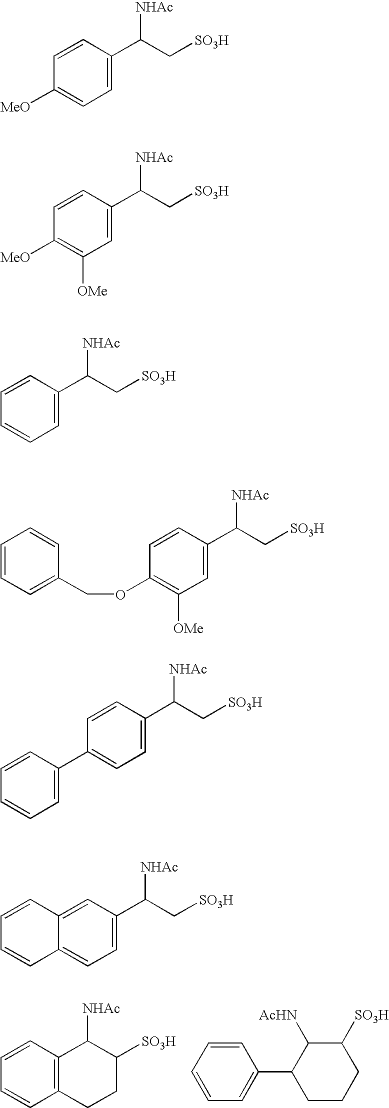
and pharmaceutically acceptable salts, prodrugs, and esters thereof.

In a further embodiment, the compound of the invention is of the Formula (III-B): 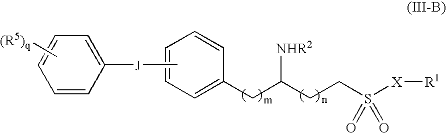
wherein:

X is oxygen or nitrogen;

m and n are each independently 0, 1, 2, 3, 4, 5, 6, 7, 8, 9 or 10;

q is 1, 2, 3, 4, or 5;

R¹ is hydrogen, metal ion, alkyl, mercaptoalkyl, alkenyl, alkynyl, cycloalkyl, aryl, or a moiety together with X to form a natural or unnatural amino acid residue, or —(CH₂)ₚ—Y;

Y is hydrogen or a heterocyclic moiety selected from the group consisting of thiazolyl, triazolyl, tetrazolyl, amidino, imidazolyl, benzothiazolyl, and benzoimidazolyl;

p is 0, 1, 2, 3, or 4;

R² is hydrogen, alkyl, mercaptoalkyl, alkenyl, alkynyl, cycloalkyl, aryl, alkylcarbonyl, arylcarbonyl, or alkoxycarbonyl;

R⁵ is selected from the group consisting of hydrogen, halogen, amino, nitro, hydroxy, carbonyl, thiol, carboxy, alkyl, alkoxy, alkoxycarbonyl, acyl, alkylamino, acylamino;

q is an integer selected from 1 to 5;

J is absent, oxygen, nitrogen, sulfur, or a divalent link-moiety comsisting of, without limiting to, lower alkylene, alkylenyloxy, alkylenylamino, alkylenylthio, alkylenyloxyalkyl, alkylenylamonialkyl, alkylenylthioalkyl, alkenyl, alkenyloxy, alkenylamino, or alkenylthio; and pharmaceutically acceptable salts, esters, and prodrugs thereof.

In yet another embodiment, the compound of the invention is:.

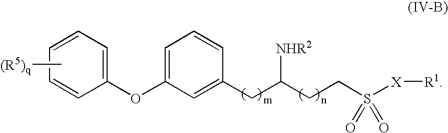

In a further embodiment, m is 0.

Additional examples of compounds that can be used the invention include: 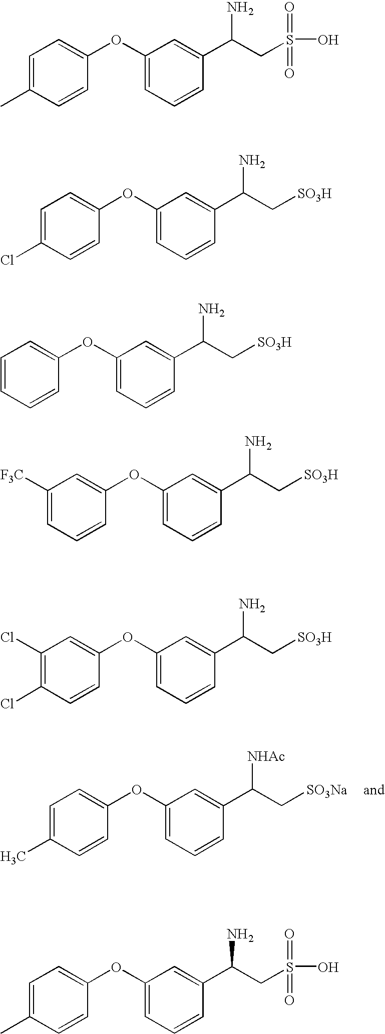
and pharmaceutically acceptable salts, esters and prodrugs thereof.

Further examples of compounds of the invention include compounds of the Table 2.

TABLE 2

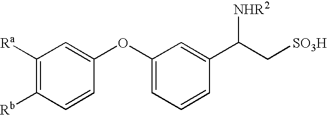

| No. in Series | Configuration | R² | R^{a} | R^{b} |
|---|---|---|---|---|
| 1 | D,L- | H | H | H |
| 2 | D,L- | H | H | Cl |
| 3 | D,L- | H | Cl | Cl |
| 4 | D,L- | H | H | CH₃ |
| 5 | D,L- | H | CF₃ | H |
| 6 | D- | H | H | H |
| 7 | D- | H | H | Cl |
| 8 | D- | H | Cl | Cl |
| 9 | D- | H | H | CH₃ |
| 10 | D- | H | CF₃ | H |
| 11 | L- | H | H | H |
| 12 | L- | H | H | Cl |
| 13 | L- | H | Cl | Cl |
| 14 | L- | H | H | CH₃ |
| 15 | L- | H | CF₃ | H |
| 16 | D,L- | Ac | H | H |
| 17 | D,L- | Ac | H | Cl |
| 18 | D,L- | Ac | Cl | Cl |
| 19 | D,L- | Ac | H | CH₃ |
| 20 | D,L- | Ac | CF₃ | H |
| 21 | D- | Ac | H | H |
| 22 | D- | Ac | H | Cl |
| 23 | D- | Ac | Cl | Cl |
| 24 | D- | Ac | H | CH₃ |
| 25 | D- | Ac | CF₃ | H |
| 26 | L- | Ac | H | H |
| 27 | L- | Ac | H | Cl |
| 28 | L- | Ac | Cl | Cl |
| 29 | L- | Ac | H | CH₃ |
| 30 | L- | Ac | CF₃ | H |

In another embodiment, the invention includes the use of compounds of Formula (V-B): 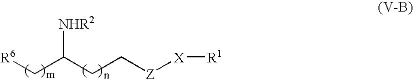
wherein:

R⁶ is a substituted or unsubstituted heterocyclic moiety.

In a further embodiment, m is 0 or 1. In another embodiment, n is 0 or 1. In another further embodiment, R⁶ is thiazolyl, oxazoylyl, pyrazolyl, indolyl, pyridinyl, thiazinyl, thiophenyl, benzothiophenyl, dihydroimidazolyl, dihydrothiazolyl, oxazolidinyl, thiazolidinyl, tetrahydropyrimidinyl, or oxazinyl. In yet another embodiment, Z is S(O)₂.

In a further embodiment, the invention pertains to use of the following compounds: 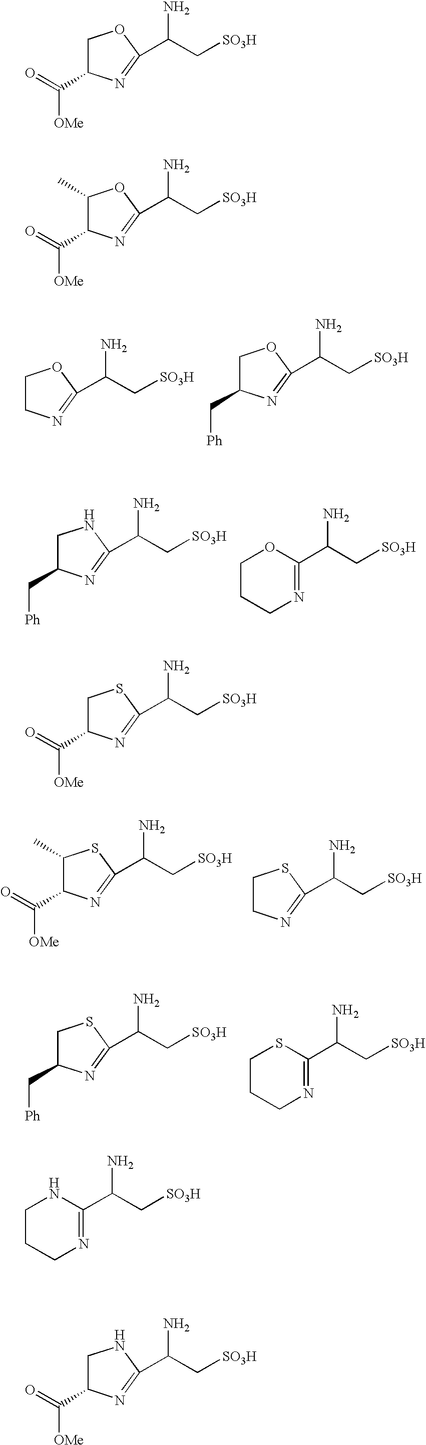 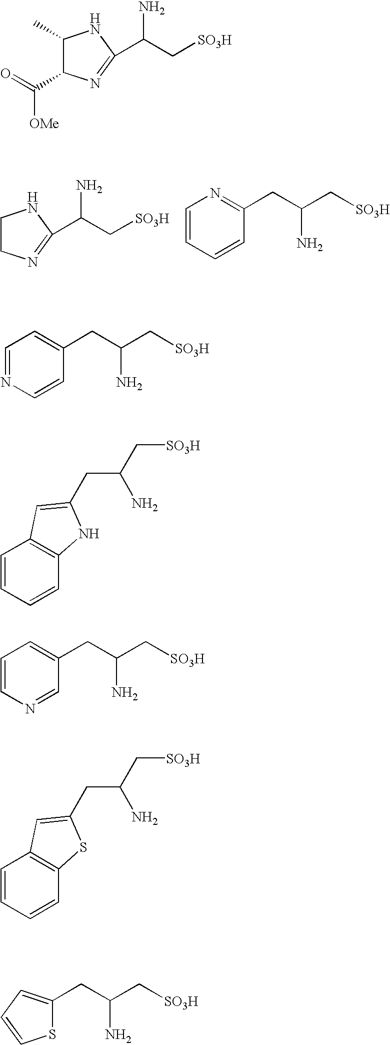
and pharmaceutically acceptable salts, esters and prodrugs thereof.

In another aspect, the invention relates to pharmaceutical compositions comprising an alkylsulfonic acid and another drug that targets secondary symptoms of Alzheimer's disease, such as behavioral and emotional difficulties. For example, some approved medications exist that appear to improve memory and cognition, but do not address the underlying pathology, as discussed more fully elsewhere herein.

Those skilled in the art will recognize, or be able to ascertain using no more than routine experimentation, numerous equivalents to the specific procedures, embodiments, claims, and examples described herein. Such equivalents were considered to be within the scope of this invention and covered by the claims appended hereto. The contents of all references, issued patents, and published patent applications cited throughout this application are hereby incorporated by reference. The invention is further illustrated by the following example, which should not be construed as further limiting.

### EXAMPLES

Binding and Antifibrillogenic Assays

Test compounds were purchased from commercial sources or synthesized and screened by mass spectroscopy (“MS”) assays. The MS assay gives data on the ability of compounds to bind to an amyloid.

In the mass spectroscopy (“MS”) assay, samples were prepared as aqueous solutions containing 20% ethanol, 200 μM of a test compound and 20 μM of solubilized Aβ40. The pH value of each sample was adjusted to 7.4 (±0.2) by addition of 0.1% aqueous sodium hydroxide. The solutions were then analyzed by electrospray ionization mass spectroscopy using a Waters ZQ 4000 mass spectrometer. Samples were introduced by direct infusion at a flow-rate of 25 ηL/minute within 2 hours after sample preparation. The source temperature was kept at 70° C. and the cone voltage was 20 V for all the analysis. Data were processed using Masslynx 3.5 software. The MS assay gives data on the ability of compounds to bind to Aβ, whereas the ThT, EM and CD assays give data on inhibition of fibrillogenesis. The results from the assay for binding to Aβ are summarized in Table 3. “+++” indicates strong binding; “++” indicates moderate binding; “+” indicates weak binding; “−” indicates no detectable binding; and entries left blank were not determined.

An ultraviolet absorption assay is also available, and this assay gives an indication of the ability of test compounds to bind to (fibrillar) Aβ. The experiments were carried out in a blinded fashion. Test compound at 20 μM was incubated with 50 μM Aβ(1-40) fibers for 1 h at 37° C. in Tris buffered saline (20 mM Tris, 150 mM NaCl, pH 7.4 containing 0.01 sodium azide). Following incubation, the solution was centrifuged for 20 minutes at 21,000 g to sediment the Aβ(1-40) fibers along with any bound test compound. The amount of test compound remaining in the supernatant-was determined by reading the absorbance. The fraction of test compound bound was then calculated by comparing the amount remaining in the supernatants of incubations with Aβ to the amount remaining in control incubations that do not contain Aβ fibers. Thioflavin T and Congo Red, both of which are known to bind to Aβ fibers, are included in each assay run as positive controls. Before assaying, test compounds were diluted to 40 μM, which is twice the concentration in the final test, and then scanned using the Hewlett Packard 8453 UV/VIS spectrophotometer to determine if the absorbance was sufficient for detection.

TABLE 3
Relative Binding Affinities of Compounds of the Invention

| | MS Binding | |
|---|---|---|
| ID | Aβ1-40 | Structure/Name of Compound |
| A | ++ | 5-phenyl-1-sulfopropyl-1,2,3,6-tetrahydropyridine |
| | | 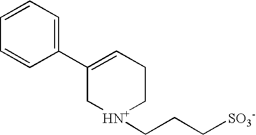 |
| B | + | 2-phenyl-1-sulfopropyl-1,2,3,6-tetrahydropyridine |
| | | 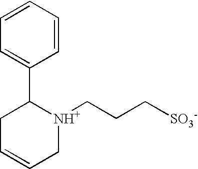 |
| C | + | 4-(3-phenylpropyl)-1-sulfopropylpyridine |
| | | 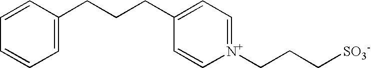 |
| D | + | 4-(3-phenylpropyl)-1-sulfopropyl-2,3,6-tetrahydropyridine |
| | | 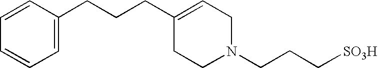 |
| E | | 3-(4-cyano-4-phenylpiperidin-1-yl)-1-propanesulfonlc acid |
| | | 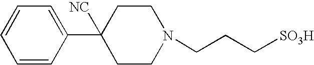 |
| F | | 3[(4-(4-fluorophenyl)-1,2,3,6-tetrahydropyridin-1-yl]- |
| | | 1-propanesulfonic acid |
| | | 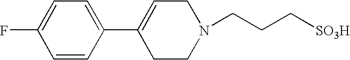 |
| G | | 3-(4-(4-bromophenyl)-4-hydroxyplperidin-1-yl]- |
| | | 1-propanesulfonic acid |
| | | 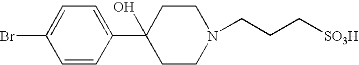 |
| H | | 3-[4-(4-chlorophennyl)-4-hydroxypiperidin-1-yl]- |
| | | 1-propanesulfonic acid |
| | | 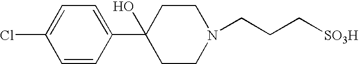 |
| I | | 3-(4-acetyl-4-phenylpiperidin-1-yl)-1-propanesulfonic acid |
| | | 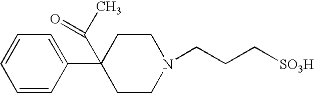 |
| J | | 3-[4-(4-chlorophenyl)-1,2,3,6-tetrahydropyridin-1-yl]- |
| | | 1-propanesulfonic acid |
| | | 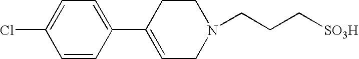 |
| K | | 3-(4-phenylpiperazin-1-yl)-1-propanesulfonic acid |
| | | 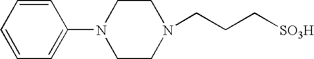 |
| L | | 3-[4-(4-chlorophenyl)piperazin-1-yl]-1-propanesulfonic acid |
| | | 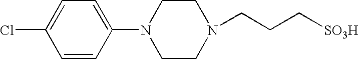 |
| M | | 3-[4-(2-fluorophenyl)piperazin-1-yl]-1-propanesulfonlc acid |
| | | 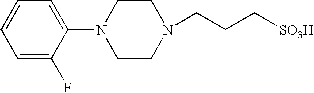 |
| N | | 3-[4-(4-nitrophenyl)piperazin-1-yl]-1-propanesulfonic acid |
| | | 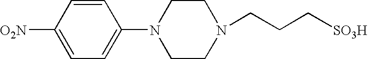 |
| O | | 3-(4-phenyl-1,2,3,6-tetrahydrapyridin-1-yl)-1-propanesulfonic acid |
| | | 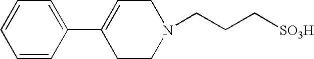 |
| P | | 3-[4-(4-fluorophenyl)piperazin-1-yl]-1-propanesulfonic acid |
| | | 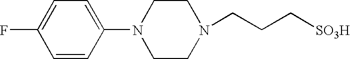 |
| Q | | 3-(4-phenyl-1,2,3,6-tetrahydropyridin-1-yl)-propanoic acid |
| | | 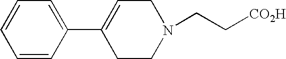 |
| R | | 3-(4-phenyl-1,2,3,6-tetrahydropyridin-1-yl)butanoic acid |
| | | hydrochloride |
| | | 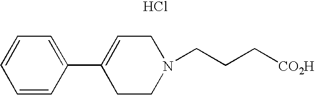 |
| S | +++ | 3-(3,4-dimethoxybenzyl) amino)-1-propanesulfonic acid |
| | | 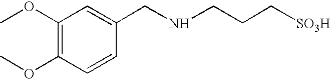 |
| T | + | 4-(4-cyclohex-3-enylpyridyl) butanesulfonic acid, inner salt |
| | | 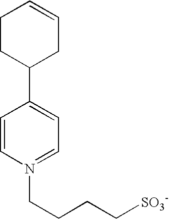 |
| U | | N-[3-(4-benzyl-1-piperidyl)-1-propanesulfonyl]-L-leucine methyl |
| | | ester |
| | | 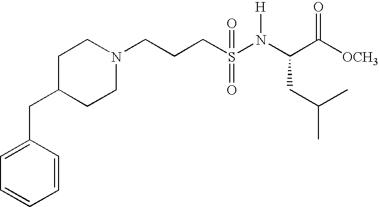 |
| V | | N-[3-(4-benzyl-1-piperazinyl)-1-propanesulfonyl]-L-leucine methyl |
| | | ester |
| | | 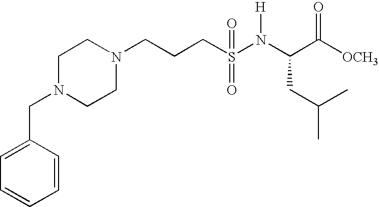 |
| X | | L-Phe-L-Phe-Taurine |
| | | 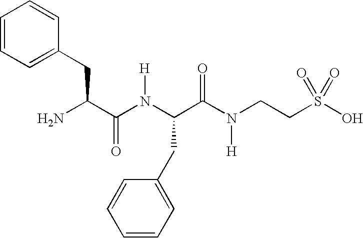 |
| Y | | N-Boc-L-Phe-homoTau-L-Phe-Oet |
| | | 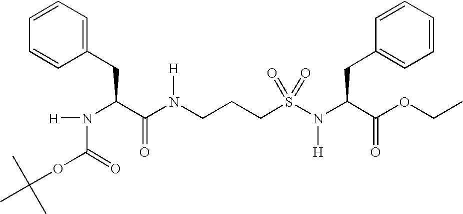 |
| Z | | L-Phe-homotau-L-Phe-OEt hydrochloride |
| | | 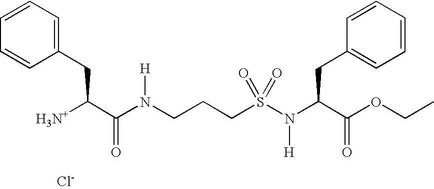 |
| AA | | N-BOC-L-Phe-homo-L-Phe-ONa |
| | | 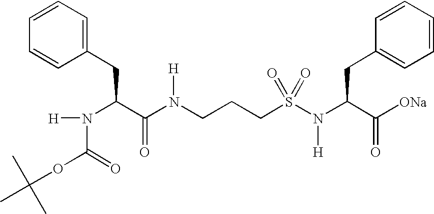 |
| AB | | L-Phe-homotau-L-Phe-OMe hydrochloride |
| | | 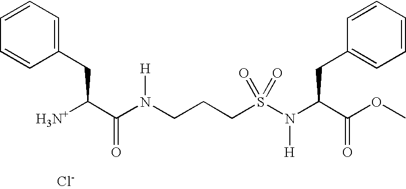 |
| AC | + | N-benzyloxycarbonyl-3-amino-2-hydroxy-1-propanesulfonic acid |
| | | sodium salt monohydrate |
| | | 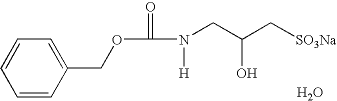 |
| AD | + | N-benzyloxycarbonyl-4-amino-1-butanesulfonic acid sodium salt |
| | | monohydrate |
| | | 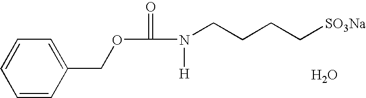 |
| AE | + | N-benzyloxycarbonyl-3-amino-1-propanesulfonic acid sodium salt |
| | | monohydrate |
| | | 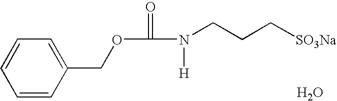 |
| AF | + | 3-{[(benzhydrylamino) carbonyl]amino}-1-propanesulfanic acid |
| | | 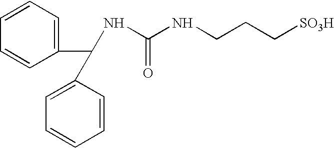 |
| AG | + | 3-[(phenylacetyl)amino]-1-propanesulfonic acid, sodium salt |
| | | 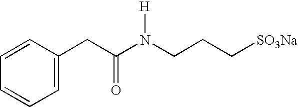 |
| AH | + | 3-{[(benzylamino) carbonyl]amino}-1-propanesulfonic acid, |
| | | sodium salt |
| | | 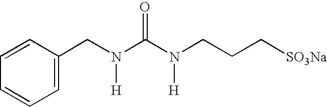 |
| AI | | 3-{[(hexylamino)carbonyl]amino}-1-propanesulfonic add, |
| | | sodium salt |
| | | 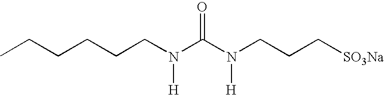 |
| AJ | | 3-{[(dodecylamino)carbonyl]amino}-1-propanesulfonic acid, |
| | | sodium salt |
| | | 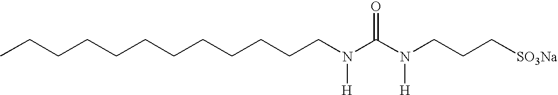 |
| AK | ++ | 3-{[(adamantylamino)carbonyl]amino}-1-propanesulfonic acid, |
| | | sodium salt |
| | | 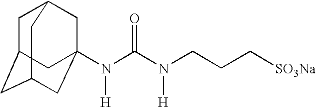 |
| AL | ++ | 3-{[2-(4-isobutylphenyl)propanoyl]amino}-1-propanesulfonic acid, |
| | | sodium salt |
| | | 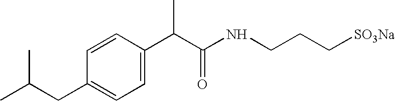 |
| AM | + | 3-{[(benzylamino)carbonothioyl]amino}-1-propanesulfonic acid |
| | | sodium salt |
| | | 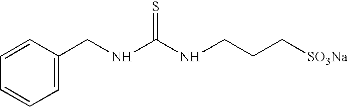 |
| AN | − | 3-((N-methylnicotlnoyl)amino)-1-propanesulfonic acid, inner salt |
| | | 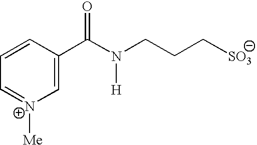 |
| AO | | 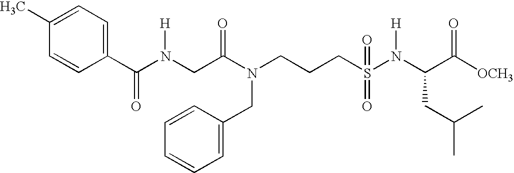 |
| AP | | 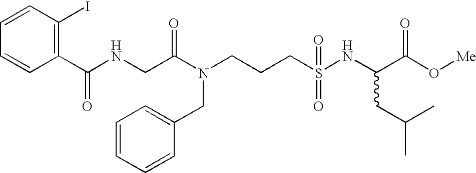 |
| AQ | | 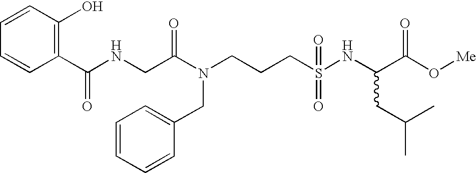 |
| AR | | 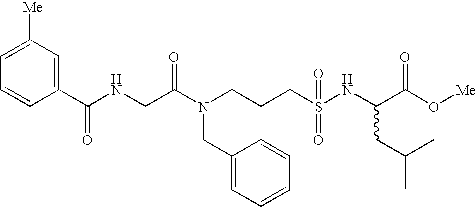 |
| AS | | 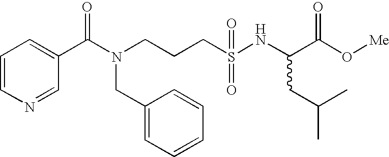 |
| AT | | 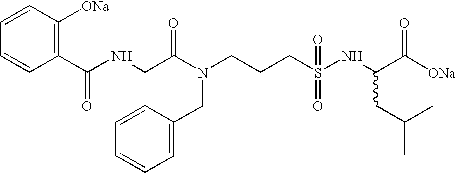 |
| AU | | N-(3-dibenzylamino-1-propanesulfonyl)-L-phenylalanine, sodium salt |
| | | 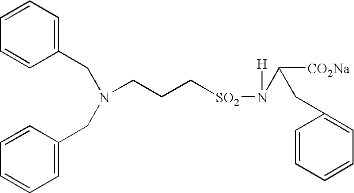 |
| AV | | 3-dibenzylamino-1-propanesulfonic acid |
| | | 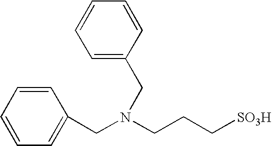 |
| AW | ++ | 3-[((1,3-benzodioxol-5-yl)methyl)amino]-1-propanesulfonic acid |
| | | 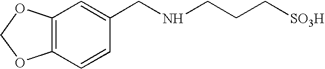 |
| AX | +++ | 3-(3,4-dimethoxybenzyl amino)-1-propanesulfonic acid |
| | | 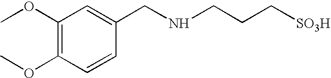 |
| AY | ++ | 3-(3,4,5-trimethoxybenzylamino)-1-propanesulfonic acid |
| | | 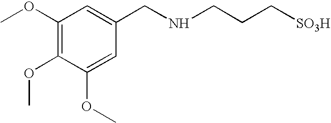 |
| AZ | ++ | 3-(2,3-dimethoxybenzylamino)-1-propanesulfonic acid |
| | | 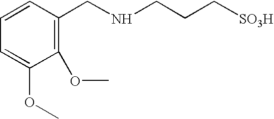 |
| BA | +++ | 3-(3,5-dimethoxybenzylamino)-1-propanesulfonic acid |
| | | 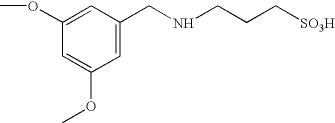 |
| BB | +++ | 3-(2,4-dimethoxybenzylamino)-1-propanesulfonic acid |
| | | 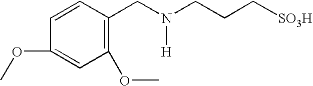 |
| BC | + | 3-(3,4-dihydroxybenzyl amino)-1-propanesulfonic acid |
| | | 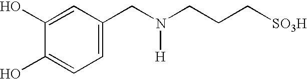 |
| BD | | N-(3-phenylamino-1-propanesulfonyl)-L-leucine |
| | | 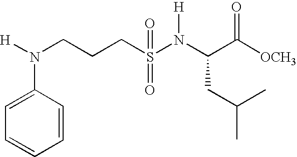 |
| BE | | 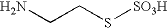 |
| BF | +++ | 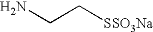 |
| BG | − | 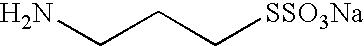 |
| BH | | 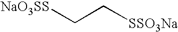 |
| BI | + | 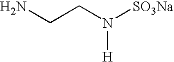 |
| BJ | | 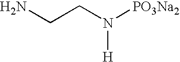 |
| BK | | 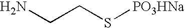 |
| BL | | 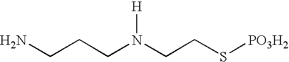 |
| BM | | 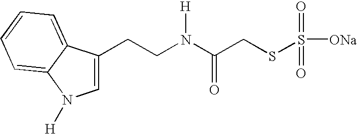 |
| BN | | 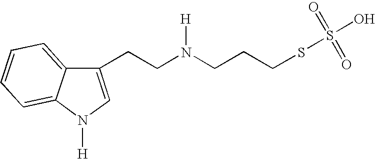 |
| BO | | 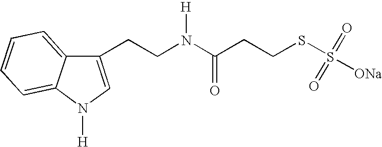 |
| BP | | 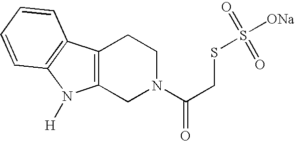 |
| BQ | | [2-(Ethoxycarbonyl)ethyl]-thiosulfonic acid, sodium salt |
| | | 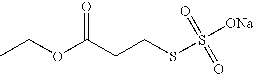 |
| BR | | [3-(Ethoxycarbonyl)propyl]-thiosulfonic acid, sodium salt |
| | | 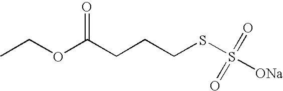 |
| BS | | S-(2-{[2-(1-benzofuran-3-yl)ethyl]amino}-2-oxoethyl)thiosulfate, |
| | | sodium salt |
| | | 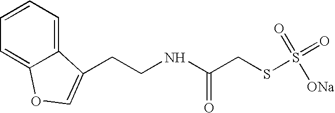 |
| BT | +++ | 3-{[2-(3-indolyl)ethyl]-amino}-1-propanesulfonic acid |
| | | 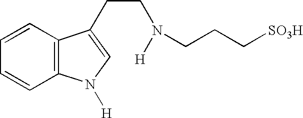 |
| BU | +++ | 3-(1,2,3,4-tetrahydro-1-naphthylamino)-1-propanesulfonic acid |
| | | 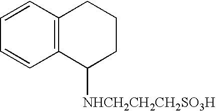 |
| BV | +++ | 3-(ethylamino)-1-propanesulfonic acid |
| | | 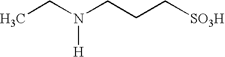 |
| BW | +++ | 3-(1-adamantyl)amino-1-propanesulfonic acid |
| | | 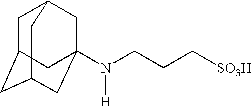 |
| BX | +++ | 3-(t-butyl)amino-1-propanesulfonic acid |
| | | 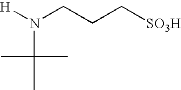 |
| BY | +++ | 3-(2-norbornyl)amino-1-propanesulfonic acid |
| | | 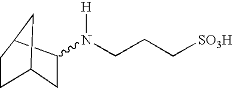 |
| BZ | +++ | 3-(2-adamantyl)amino-1-propanesulfonic acid |
| | | 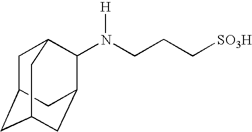 |
| CA | +++ | 3-[(dl)-1-hydroxy-2-pentyl]amino-1-propanesulfonic acid |
| | | 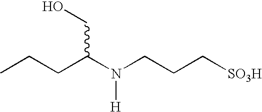 |
| CB | ++ | 4-amino-1-butanesulfonic acid |
| | | 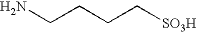 |
| CC | | 5-amino-1-pentanesulfonic add |
| | | 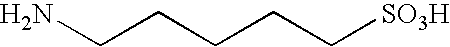 |
| CD | | 6-amino-1-hexanesulfonic acid |
| | | 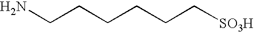 |
| CE | | 3-isobutylamino-1-propanesulfonic acid |
| | | 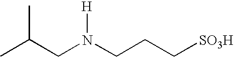 |
| CF | | 3-pentylamino-1-propanesulfonic add |
| | | 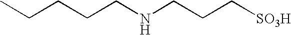 |
| CG | | 3-isopropylamino-1-propanesulfonic acid |
| | | 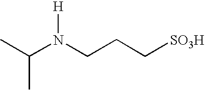 |
| CH | | 3-isoamylamino-1-propanesulfonic acid |
| | | 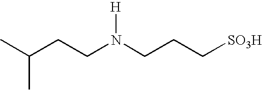 |
| CI | | 3-(cyclopropylamino)-1-propanesulfonic acid |
| | | 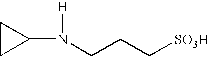 |
| CJ | | 3-(cyclopentylamino)-1-propanesulfonic acid |
| | | 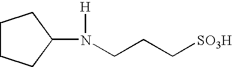 |
| CK | | 3-(cycloheptylamino)-1-propanesulfonic acid |
| | | 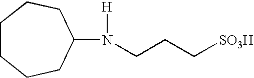 |
| CL | | 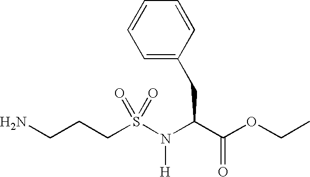 |
| CM | | cyclopentylsutfamic acid, sodium salt |
| | | 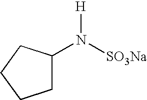 |
| CN | | cycloheptylsulfonic add, sodium salt |
| | | 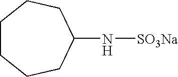 |
| CO | | 4-iodo-N-(3-sulfopropyl)-L-phenylalanine amide |
| | | 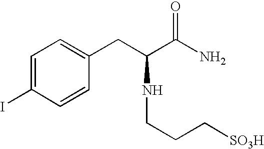 |
| CP | + | 3-(amidinothio)-1-propanesulfonic acid |
| | | 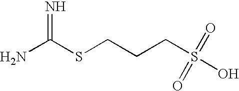 |
| CQ | | 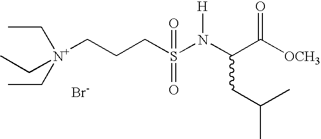 |
| CR | | 4-Aminothlophenol-S-sulfate |
| | | 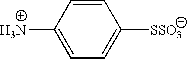 |
| CS | | 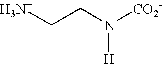 |
| CT | | N-(beta-mercaptoethyl)tryptamine hydrochloride |
| | | 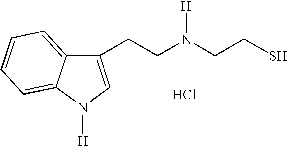 |
| CU | | (S)-3-(1-hydroxy-2-butylamino)-1-propanesulfonic acid |
| | | 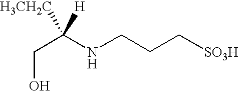 |
| CV | | 3-ethylamino-1-propanesulfonic acid |
| | | 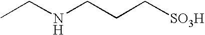 |
| CW | | 3-(1-hydroxy-2-propylamino)-1-propanesulfonic acid |
| | | 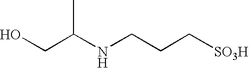 |
| CX | | 3-heptylamino-1-propane-sulfonic acid |
| | | 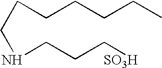 |
| CY | | 3-(3,5-dimethyl-1-adamantylamino)-1-propanesulfanic acid |
| | | 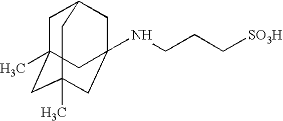 |
| CZ | | 3-octylamino-1-propanesulfonic acid |
| | | 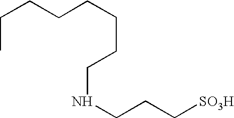 |
| DA | | 3-nonylamino-1-propanesulfonic acid |
| | | 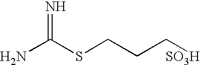 |
| DB | | 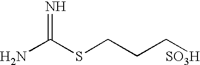 |
| DC | | 3-cyclohexylamino-2-hydroxy-1-propanesulfonic acid |
| | | 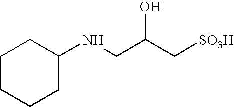 |

Aβ CSF Concentration

Method: CSF was obtained from patients before treatment as well as following 3-month treatment with test sulfonic acid first agent at daily doses of 100 mg, 200 mg, and 300 mg. CSF was fractionated by FPLC following treatment with formic acid. The Aβ containing fraction was lyophilised. Aβ was measured using an ELISA assay (Biosource). Results are expressed as in pg/ml.

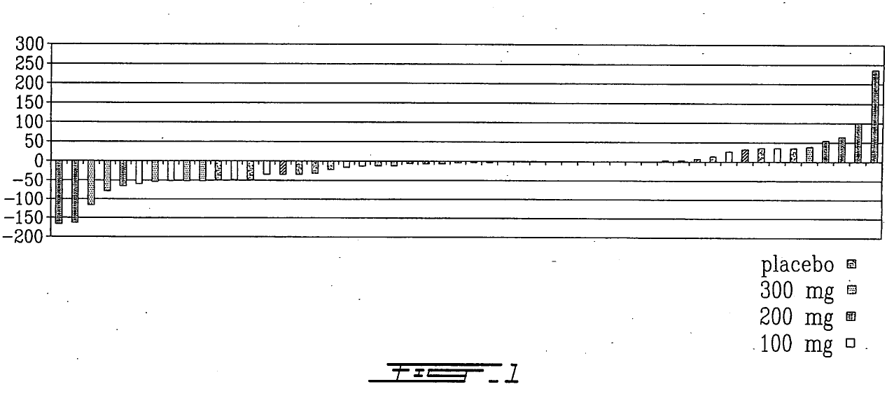

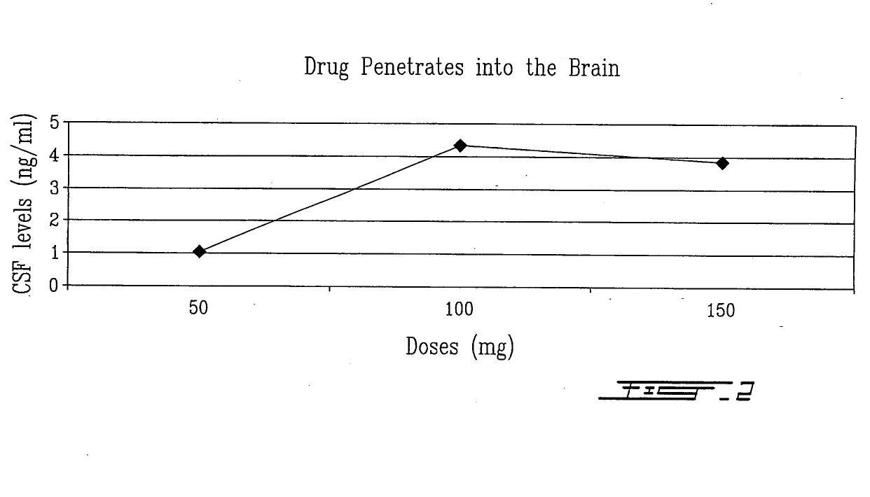

Test sulfonic acid was found to markedly reduce the CSF level of Aβ when patients were treated with 200 or 300 mg daily doses (see FIG. 1). The majority of patients on placebo and on 100 mg daily doses showed stable Aβ CSF levels over a 3-month period. Interestingly, the greatest reduction of Aβ was seen in patients taking 200 or 300 mg daily doses. Presence of a drug in the cerebrospinal fluid suggests that the drug can cross the blood brain barrier to penetrate the brain. Presence in the CSF was determined in patients having been on the drug for 3 months. CSF was collected 5 hours following dosing and levels were determined by LC-MS/MS. As is shown in FIG. 2, the drug was found to be present in CSF of patients. CSF concentration was found to be dose dependent, i.e., patients receiving 200 or 300 mg daily dosing had a greater concentration than that seen in patients with 100 mg daily dosing.

In another example, a Phase II clinical trial that was a multi-centre, randomized, double-blind, placebo-controlled, and parallel-designed study was carried out A total of 58 patients with mild-to-moderate Alzheimer's disease were randomized to receive either placebo or a drug at daily doses of 100 mg, 200 mg, or 300 mg for 12 weeks. Patients who completed the 3-month study were offered to participate in an open-label extension study for an additional 9 months. In this ongoing open label study, all patients receive 300 mg of the drug daily. The primary objectives of this Phase II clinical trial were to evaluate the safety, tolerability, and pharmacokinetics of the drug in patients with mild-to-moderate Alzheimer's disease. As is discussed below, our results show that these objectives have been met.

There were no safety findings of concern at the three doses tested. The most frequent adverse events reported on drug were nausea and vomiting and their occurrence was dose-dependent. These adverse events were usually temporary and mild-to-moderate in severity. Only 3 patients (6.7%) experienced an adverse event (i.e., nausea or weakness/weight loss) that caused them to discontinue prematurely use of the drug.

The pharmacokinetic profile of the drug was well defined in this Alzheimer's disease patient population. The extent of systemic exposure was approximately proportional to the administered dose. The bioanalysis revealed the presence of drug in the CSF of Alzheimer's disease patients and the levels appeared to be dose-related. This important finding suggests that the drug penetrates the brain.

Secondary objectives of the study include assessing the effect of the drug on levels of amyloid β (Aβ42) and tau proteins (two important biomarkers for Alzheimer's disease) in the CSF. Although the study was neither powered nor designed to detect clinical improvement in psychometric tests, tests on cognitive function (e.g., ADAS-Cog, Mini Mental State Examination (MMSE)) and a global measure of performance (Clinical Deterioration Scale Sum of Boxes (CDR-SB)) were included on an exploratory basis.

Patients who had a marked decrease of Aβ42 (≧50 pg/ml) in the CSF at the 3 month time point were all on the drug. A reduction as high as 70% was seen in the two highest dose groups. These results show that the drug has the ability to act on Aβ42 levels. This reduction is consistent with the previously reported ability of the drug to significantly decrease Aβ42 brain levels in a transgenic mouse model of brain amyloid.

Interestingly, patients in the 300 mg treatment group, with stable or declining levels of Aβ42 levels in the CSF at 3 months, and who have continued in the open-label extension study, showed a stabilization of cognitive function (ADAS-cog) at the 6-month time point. Levels of tau in the CSF after 3 months were unchanged in all treatment groups.

Cognitive function and global measure of performance was also observed. As expected for a disease-modifying therapeutic approach, the drug had no detectable effect on cognitive function or global measure of performance after 3 months of administration. However, the eight patients who received 300 mg of the drug in the ongoing open-label extension study were stable in their ADAS-cog score at the 6-month time point. This compares favorably with a 2-3 point deterioration in cognitive function test scores over the same time period, in published reports of large cohorts of control patients (Rogers et al., Arch. Intern. Med. 158, 1021-1031 (1998)).

All publications cited above are incorporated by reference herein in their entireties. Other embodiments of the invention are within the following claims.

What is claimed is:

1. A method of preventing or treating an amyloid-β related disease in a subject, said method comprising administering to a subject in need thereof an effective amount of a first agent that prevents or treats amyloid-β related disease, and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation.

2. The method of claim 1, wherein said first agent prevents or inhibits amyloid-β fibril formation, neurodegeneration, or cellular toxicity.

3. The method of claim 1, wherein said amyloid-β related disease is Alzheimer's disease, mild cognitive impairment, mild-to-moderate cognitive impairment, vascular dementia, cerebral amyloid angiopathy, hereditary cerebral hemorrhage, senile dementia, Down's syndrome, inclusion body myositis, age-related macular degeneration, or a condition associated with Alzheimer's disease.

4. The method of claim 3, wherein said Alzheimer's disease is sporadic (non-hereditary) or familial (hereditary).

5. The method of claim 3, wherein said condition-associated-with Alzheimer's disease is selected from the group consisting of hypothyroidism, cerebrovascular disease, cardiovascular disease, memory loss, anxiety, a behavioral dysfunction, a neurological condition, or a psychological condition.

6. The method of claim 5, wherein said behavioral dysfunction is apathy, aggression, or incontinence.

7. The method of claim 5, wherein said neurological condition is Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, or posthypoxic damage.

8. The method of claim 5, wherein said psychological condition is depression, delusions, illusions, hallucinations, sexual disorders, weight loss, psychosis, a sleep disturbance, insomnia, behavioral disinhibition, poor insight, suicidal ideation, depressed mood, irritability, anhedonia, social withdrawal, or excessive guilt.

9. The method of claim 1, wherein said subject has a genomic mutation in an amyloid precursor protein gene, an ApoE gene, or a presenilin gene.

10. The method of claim 1, wherein said subject has amyloid-β deposits.

11. The method of claim 1, wherein said subject is a human.

12. The method of claim 1, wherein said amyloid-β is an amyloidogenic peptide produced from β-amyloid precursor protein.

13. The method of claim 12, wherein said β-amyloid is a peptide having 39-43 amino acids.

14. The method of claim 1, wherein said first agent prevents or inhibits β-amyloid fibril formation; prevents β-amyloid peptide, in its soluble, oligomeric form, or in its fibrillar form, from binding or adhering to a cell surface and causing cell damage or toxicity; blocks amyloid-induced cellular toxicity or microglial activation; blocks amyloid-induced neurotoxicity; reduces the rate or amount of β-amyloid aggregation, fibril formation, or deposition; slows the rate of amyloid-β fibril formation or deposition; lessens the degree of amyloid-β deposition; inhibits, reduces, or prevents amyloid-β fibril formation; inhibits amyloidβ, induced inflammation; enhances the clearance of amyloid-β from the brain; alters the equilibrium of amyloid-β between the cerebrospinal fluid or brain and the plasma and decreases the amount of amyloid-β in the brain versus the equilibrium distribution in an untreated subject; reverses or favors deposition of amyloid in a subject having amyloid deposits; favors plaque clearance or slows deposition in a subject having amyloid deposits; decreases the amyloid-β concentration in the brain of a subject versus an untreated subject; penetrates into the brain; maintains soluble amyloid in a non-fibrillar form; increases the rate of clearance of soluble amyloid from the brain of a subject versus an untreated subject; or inhibits or reduces an interation between amyloid-β and a cell surface constituent.

15. The method of claim 1, wherein said first agent is a substituted or unsubstituted alkylsulfonic acid, a substituted or unsubstituted alkylsulfuric acid, a substituted or unsubstituted alkylthiosulfonic acid, a substituted or unsubstituted alkylthiosulfuric acid, a substituted or unsubstituted lower alkylsulfonic acid, a (substituted- or unsubstituted-amino)-substituted alkylsulfonic acid, a (substituted- or unsubstituted-amino)-substituted lower alkylsulfonic acid, a substituted or unsubstituted straight-chain alkylsulfonic acid, a substituted or unsubstituted cycloalkylsulfonic acid, a substituted or unsubstituted branched-chain alkylsulfonic acid, or an ester or amide thereof, including pharmaceutically acceptable salts thereof.

16. The method of claim 15, wherein said amino substituent has the formula —NR^{a}R^{b}, wherein R^{a} and R^{b} are each independently hydrogen, an alkyl group, an aryl group, or a heterocyclyl group, or R^{a} and R^{b}, taken together with the nitrogen atom to which they are attached, form a heterocyclic moiety having from 3 to 8 atoms in the ring.

17. The method of claim 16, wherein said heterocyclic moiety comprises a piperidinyl or pyrrolidinyl group.

18. The method of claim 15, wherein said amino substituent comprises an alkylamino or dialkylamino group.

19. The method of claim 15, wherein said alkylsulfonic acid comprises an alkyl group substituted with at least a group of the formula —SO₃H or —SO₃⁻X⁺, where X⁺ is a cationic group at physiologic pH.

20. The method of claim 19, wherein said cationic group is a hydrogen atom, a sodium atom, or an amino group.

21. The method of claim 15, wherein said alkylsulfonic acid is substituted with a straight or branched alkyl or cycloalkyl group, or a group of the formula —NH₂, —SO₃H, —OSO₃H, —CN, —NO₂, —F, —Cl, —Br, —I, —CH₂OCH₃, —OCH₃, —SH, —SCH₃, —OH, or —CO₂H.

22. The method of claim 15, wherein said alkylsulfonic acid is substituted with a substituent selected from the group consisting of halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl groups.

23. The method of claim 15, wherein said first agent is a compound or mixture of compounds having the following structure, 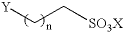
where Y is —NR^{a}R^{b} or —SO₃⁻X⁺, n is an integer from 1 to 5, and X⁺ is hydrogen or a cationic group.

24. The method of claim 23, wherein said first agent is a compound or mixture of compounds having one of the following structures 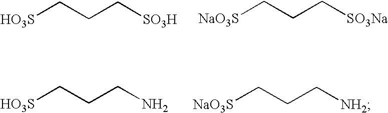
or a pharmaceutically acceptable salt thereof.

25. The method of claim 15, wherein said first agent is 3-amino-1-propanesulfonic acid or a pharmaceutically acceptable salt thereof

26. The method of claim 1, wherein said first and second agents are administered to said subject together, in a single pharmaceutical composition.

27. The method of claim 1, wherein said first and second agents are administered sequentially.

28. The method of claim 1, wherein at least one of said first and said second agents is orally administered to said subject.

29. The method of claim 1, wherein said second agent is a peptide or peptidomimetic compound that reduces or inhibits amyloid-β fibril formation.

30. The method of claim 29, wherein said peptide comprises hydrophobic amino acids and binds to the hydrophobic region of an amyloid-β peptide, thus blocking β-amyloid fibril formation.

31. The method of claim 30, wherein said peptide comprises one or more modifying groups that enhance the ability of the peptide to block amyloid fibril formation.

32. The method of claim 1, wherein said peptide is an all D peptide.

33. The method of claim 1, wherein said peptide or peptidomimetic is administered to induce a prophylactic or therapeutic immune response against amyloid-β fibril formation, said method further comprising administration of an adjuvant

34. The method of claim 1, wherein said immune system modulator is selected from the group consisting of antibodies, antibody fragments, T-cells, B-cells, NK cells, NKT cells, dendritic cells, macrophages, basophils, monocytes, and components of the complement pathway.

35. The method of claim 1, further comprising administration of a third agent.

36. The method of claim 35, wherein said third agent is selected from the group consisting of an adrenergic, anti-adrenergic, anti-androgen, anti-anginal, anti-anxiety, anticonvulsant, antidepressant, anti-epileptic, antihyperlipidemic, antihypertipoproteinemic, antihypertensive, anti-inflammatory, antiobessional, antiparkinsonian, antipsychotic, adrenocortical steroid; adrenocortical suppressant; aldosterone antagonist; amino acid; anabolic steroid; analeptic; androgen; blood glucose regulator, cardioprotectant; cardiovascular; cholinergic agonist or antagonist; cholinesterase deactivator or inhibitor; cognition adjuvant or enhancer, dopaminergic; enzyme inhibitor; estrogen, free oxygen radical scavenger, GABA agonist; glutamate antagonist; hormone; hypocholesterolemic; hypolipidemic; hypotensive; immunizing; immunostimulant; monoamine oxidase inhibitor, neuroprotectve; NMDA antagonist; AMPA antagonist, competitive or non-competitive NMDA antagonist; opioid antagonist; potassium channel opener; non-hormonal sterol derivative; post-stroke and post-head trauma treatment; prostaglandin; psychotropic; relaxant; sedative; sedative-hypnotic; selective adenosine antagonist; serotonin antagonist; serotonin inhibitor; selective serotonin uptake inhibitor, serotonin receptor antagonist; sodium and calcium channel blocker; steroid; stimulant; and thyroid hormone or inhibitor agents.

37. The method of claim 1, wherein the concentration of amyloid-β or tau in the cerebrospinal fluid of said subject changes as compared to the concentration in the cerebrospinal fluid of an untreated subject or the treated subject prior to treatment.

38. The method: of claim 1, wherein the level of amyloid-β peptides in the plasma of said subject are modulated as compared to the level in the plasma of an untreated subject or the treated subject prior to treatment.

39. The method of claim 1, wherein the level of amyloid-β peptides in the cerebrospinal fluid of said subject are lowered as compared to the level in an untreated subject or the treated subject prior to treatment.

40. A method of preventing or treating an amyloid-β related disease in a subject, said method comprising administering to a subject in need thereof an effective amount of 3-amino-1-propanesulfonic acid, and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation.

41. A pharmaceutical composition for treating a subject comprising a first agent that prevents or treats amyloid-β related disease, and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation.

42. The pharmaceutical composition of claim 41, wherein said first agent prevents or inhibits amyloid-β fibril formation, neurodegeneration, or cellular toxicity.

43. The pharmaceutical composition of claim 41, wherein said first agent and said second agent are packaged in separate containers for sale or delivery to consumers.

44. The pharmaceutical composition of claim 41, wherein said first agent and said second agent are dissolved in a liquid pharmaceutically acceptable carrier or are present as a homogenous mixture in a capsule or pill.

45. The pharmaceutical composition of claim 41, further comprising a compound that increases the cerebral bioavailability of either said first agent or said second agent.

46. The pharmaceutical composition of claim 41, wherein said amyloid-β related disease is Alzheimer's disease, mild cognitive impairment, mild-to-moderate cognitive impairment, vascular dementia, cerebral amyloid angiopathy, hereditary cerebral hemorrhage, senile dementia, Down's syndrome, inclusion body myositis, age-related macular degeneration, or a condition associated with Alzheimer's disease.

47. The pharmaceutical composition of claim 46, wherein said Alzheimer's disease is sporadic (non-hereditary) or familial (hereditary).

48. The pharmaceutical composition of claim 46, wherein said condition associated with Alzheimer's disease is selected from the group consisting of hypothyroidism, cerebrovascular disease, cardiovascular disease, memory loss, anxiety, a behavioral dysfunction, a neurological condition, or a psychological condition.

49. The pharmaceutical composition of claim 48, wherein said behavioral dysfunction is apathy, aggression, or incontinence.

50. The pharmaceutical composition of the claim 48, wherein said neurological condition is Huntington's disease, amyotrophic lateral sclerosis, acquired immunodeficiency, Parkinson's disease, aphasia, apraxia, agnosia, Pick disease, dementia with Lewy bodies, altered muscle tone, seizures, sensory loss, visual field deficits, incoordination, gait disturbance, transient ischemic attack or stroke, transient alertness, attention deficit, frequent falls, syncope, neuroleptic sensitivity, normal pressure hydrocephalus, subdural hematoma, brain tumor, posttraumatic brain injury, or posthypoxic damage.

51. The pharmaceutical composition of claim 48, wherein said psychological condition is depression, delusions, illusions, hallucinations, sexual disorders, weight loss, psychosis, a sleep disturbance, insomnia, behavioral disinhibition, poor insight, suicidal ideation, depressed mood, irritability, anhedonia, social withdrawal, or excessive guilt.

52. The pharmaceutical composition of claim 41, wherein said subject has a genomic mutation in an amyloid precursor protein gene, an ApoE gene, or a presenilin gene.

53. The pharmaceutical composition of claim 41, wherein said subject has amyloid-β deposits.

54. The pharmaceutical composition of claim 41, wherein said subject is a human.

55. The pharmaceutical composition of claim 41, wherein said amyloid-β is an amyloidogenic peptide produced from β-amyloid precursor protein.

56. The pharmaceutical composition of claim 55, wherein said β-amyloid is a peptide having 39-43 amino acids.

57. The pharmaceutical composition of claim 41, wherein said first agent prevents or inhibits β-amyloid fibril formation; prevents β-amyloid peptide, in its soluble, oligomeric form, or in its fibrillar form, from binding or adhering to a cell surface and causing cell damage or toxicity, blocks amyloid-induced cellular toxicity or microglial activation; blocks amyloid-induced neurotoxicity; reduces the rate or amount of β-amyloid aggregation, fibril formation, or deposition; slows the rate of amyloid-β fibril formation or deposition; lessens the degree of amyloid-β deposition; inhibits, reduces, or prevents amyloid-β fibril formation; inhibits amyloid-β induced inflammation; enhances the clearance of amyloid-β from the brain; alters the equilibrium of amyloid-β between the cerebrospinal fluid or brain and the plasma and decreases the amount of amyloid-β in the brain versus the equilibrium distribution in an untreated subject; reverses or favors deposition of amyloid in a subject having amyloid deposits; favors plaque clearance or slows deposition in a subject having amyloid deposits; decreases the amyloid-β concentration in the brain of a subject versus an untreated subject; penetrates into the brain, maintains soluble amyloid in a non-fibrillar form; increases the rate of clearance of soluble amyloid from the brain of a subject versus an untreated subject; or inhibits or reduces an interation between amyloid-β and a cell surface constituent.

58. The pharmaceutical composition of claim 41, wherein said first agent is a substituted or unsubstituted alkylsulfonic acid, a substituted or unsubstituted alkylsulfuric acid, a substituted or unsubstituted alkylthiosulfonic acid, a substituted or unsubstituted alkylthiosulfuric acid, a substituted or unsubstituted lower alkylsulfonic acid, a (substituted- or unsubstituted-amino)-substituted alkylsulfonic acid, a (substituted- or unsubstituted-amino)-substituted lower alkylsulfonic acid, a substituted or unsubstituted straight-chain alkylsulfonic acid, a substituted or unsubstituted cycloalkylsulfonic acid, a substituted or unsubstituted branched-chain alkylsulfonic acid, or an ester or amide thereof, including pharmaceutically acceptable salts: thereof.

59. The pharmaceutical composition of claim 58, wherein said amino substituent has the formula —NR^{a}R^{b}, wherein R^{a} and R^{b} are each independently hydrogen, an alkyl group, an aryl group, or a heterocyclyl group, or R^{a} and R^{b}, taken together with the nitrogen atom to which they are attached, form a heterocyclic moiety having from 3 to 8 atoms in the ring.

60. The pharmaceutical composition of claim 59, wherein said heterocyclic moiety comprises a piperidinyl or pyrrolidinyl group.

61. The pharmaceutical composition of claim 58, wherein said amino substituent comprises an alkylamino or dialkylamino group.

62. The pharmaceutical composition of claim 58, wherein said alkylsulfonic acid comprises an alkyl group substituted with at least a group of the formula —SO₃H or —SO₃⁻X⁺, where X⁺ is a cationic group at physiologic pH.

63. The pharmaceutical composition of claim 62, wherein said cationic group is a hydrogen atom, a sodium atom, or an amino group.

64. The pharmaceutical composition of claim 58, wherein said alkylsulfonic acid is substituted with a straight or branched alkyl or cycloalkyl group, or a group of the formula —NH₂, —SO₃H, —OSO₃H, —CN, —NO₂, —F, —Cl, —Br, —I, —CH₂OCH₃, —OCH₃, —SH, —SCH₃, —OH, or —CO₂H.

65. The pharmaceutical composition of claim 58, wherein said alkylsulfonic acid is substituted with a substituent selected from the group consisting of halogeno, trifluoromethyl, nitro, cyano, C₁-C₆ alkyl, C₂-C₆ alkenyl, C₂-C₆ alkynyl, C₁-C₆ alkylcarbonyloxy, arylcarbonyloxy, C₁-C₆ alkoxycarbonyloxy, aryloxycarbonyloxy, C₁-C₆ alkylcarbonyl, C₁-C₆ alkoxycarbonyl, C₁-C₆ alkoxy, C₁-C₆ alkylthio, arylthio, heterocyclyl, aralkyl, and aryl groups.

66. The pharmaceutical composition of claim 58, wherein said first agent is a compound or mixture of compounds having the following structure 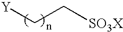
where Y is —NR^{a}R^{b} or —SO₃⁻X⁺, n is an integer from 1 to 5, and X+is hydrogen or a cationic group.

67. The pharmaceutical composition of claim 66, wherein said first agent is a compound or mixture of compounds having one of the following structures 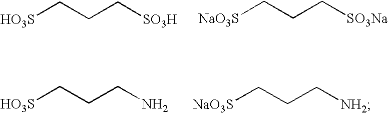
or a pharmaceutically acceptable salt thereof.

68. The pharmaceutical composition of claim 58, wherein said first agent is 3-amino-1-propanesulfonic acid or a pharmaceutically acceptable salt thereof.

69. The pharmaceutical composition of claim 58, wherein said second agent is a peptide or peptidomimetic compound that reduces or inhibits amyloid-β fibril formation.

70. The pharmaceutical composition of claim 69, wherein said peptide comprises hydrophobic amino acids and binds to the hydrophobic region of an amyloid-β peptide, thus blocking β-amyloid fibril formation.

71. The pharmaceutical composition of claim 70, wherein said peptide comprises one or more modifying groups that enhance the ability of the peptide to block amyloid fibril formation.

72. The pharmaceutical composition of claim 58, wherein said peptide is an all D peptide.

73. The pharmaceutical composition of claim 58, wherein said peptide or peptidomimetic is administered to induce a prophylactic or therapeutic immune response against amyloid-β fibril formation, and said composition further comprises an adjuvant.

74. The pharmaceutical composition of claim 58, wherein said immune system modulator is selected from the group consisting of antibodies, antibody fragments, T-cells, B-cells, NK cells, NKT cells, dendritic cells, macrophages, basophils, monocytes, and components of the complement pathway.

75. The pharmaceutical composition of claim 58, further comprising a third agent.

76. The pharmaceutical composition of claim 75, wherein said third agent is selected from the group consisting of an adrenergic, anti-adrenergic, anti-androgen, anti-anginal, anti-anxiety, anticonvulsant, antidepressant, anti-epileptic, antihyperlipidemic, antihyperlipoproteinemic, antibypertensive, anti-inflammatory, antiobessional, antiparkinsonian, antipsychotic, adrenocortical steroid; adrenocortical suppressant; aldosterone antagonist; amino acid; anabolic steroid; analeptic; androgen; blood glucose regulator; cardioprotectant; cardiovascular; cholinergic agonist or antagonist; cholinesterase deactivator or inhibitor; cognition adjuvant or enhancer, dopaminergic; enzyme inhibitor; estrogen, free oxygen radical scavenger; GABA agonist; glutamate antagonist; hormone; hypocholesterolemic; hypolipidemic; hypotensive; immunizing; immunostimulant; monoamine oxidase inhibitor, neuroprotective; NMDA antagonist; AMPA antagonist, competitive or non-competitive NMDA antagonist; opioid antagonist; potassium channel opener; non-hormonal sterol derivative; post-stroke and post-head trauma treatment; prostaglandin; psychotropic; relaxant; sedative; sedative-hypnotic; selective adenosine antagonist; serotonin antagonist; serotonin inhibitor; selective serotonin uptake inhibitor; serotonin receptor antagonist; sodium and calcium channel blocker; steroid; stimulant; and thyroid hormone or inhibitor agents.

77. A pharmaceutical composition comprising an effective amount of 3-amino-1-propanesulfonic acid, and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation.

78. A kit comprising a first agent that prevents or treats amyloid-β related disease, and a second agent that is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation.

79. The use of a first agent and a second agent in the preparation of a pharmaceutical composition for the treatment or prevention of an amyloid-β disease, wherein said first agent prevents or treats amyloid-β related disease, and said second agent is (i) a peptide or peptidomimetic that modulates amyloid-β fibril formation or induces a prophylactic or therapeutic immune response against amyloid-β fibril formation, or (ii) an immune system modulator that prevents or inhibits amyloid-β fibril formation.
